SEQUENCE LISTING (37 sequences)

SEQ ID NO: 1 - protein, 403 aa, Homo sapiens [sequence omitted]

SEQ ID NO: 2 - DNA, 8718 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcattgtgtgggtgttttattaaatgagagtttataattcaaattgcttaagtccattgaagttttaattaatgggcagccaaatgtgaatacaaagttttcagtttttttttttcctgctgtccttcaaagcctactgtttaaaaaaaaaaaaaaaaaaaaacatggcctgagagtagagtatctgtctactcatgtttaattaaggaaaaacacttatttttagggctttagtcatcacttcataaattgtataagcacattaaatagcgttctagtcctgaaaaagtccaagattcttagaaaattgtgcatatttttattatgacagatgtttgaagataattccccagaatggatttgatactttagatttcaattttgtggcttttgtctattattctgtactctgccatcagcatatggaaagcttcatttactcatcatgacttgtgccatataaaaattgatatttcggaatagtctaaaggactttttgtacttgaatttaatcatgttgtttctaatattcttaaaagcttgaagactaaagcatatcctttcaacaaagcatagtaaggtaataagaaagtgtagtttgtacaagtgttaaaaaaataaagtagacaatgttacagtgggacttattatttcaagtttacattttctccatgtaattttttaaaaagtaaatgaaaaaatgtgcaataatgtaaaatatgaagtgtatgtgtacacacattttatttttcggtatcttgggtatacgtatggttgaaaactatactggagtctaaaagtattctaatttataagaagacattttggtgatgtttgaaaaatagaaatgtgctagttttgtttttatatcatgtcctttgtacgttgtaatatgagctggcttggttcagtaaatgccatcaccatttccattgagaatttaaaactcaccagtgtttaatatgcaggcttccaaaggcttatgaaaaaaatcaagacccttaaatctagttaatttgctgctaacatgaaactctttggttcttttatttttgccagataattagacacacatctaaagcttagtcttaaatggcttaagtgtagctattgattagtgctgttgctagttcagaaagaaatgtttgtgaatggaaacaagaatattcagtccaaactgttgtaaggacagtacctgaaaaccaggaaacaggataatggaaaaagtcttttaaagatgaaatgttggagccaactttcttatagaattaattgtatgtggctatagaaagcctaatgattgttgcttatttttgagagcatattattcttttatgaccataatcttgctgtttttccatcttccaaaagatcttccttctaatatgtatatcagaatgtgggtagccagtcagacaaattcatattggttggtagctttaaaaagtttgtaatgtgaagacaggaaaggacaaaatagtttgctttggtggtagtactctggttgttaagctaggtattttgagactacttccccatcacaacaacaataaaataatcactcataatcctatcacctggagacatagccatcgttaatatgttagtgactatacaatcatgttttcttctgtatatccatgtatattctttaaaaatgaaatttatactgtacctgatctcaaagctttttagcttagtatatctgtcatgaatttgtaggatgttccattgcatcagaaaacggacagtgatttgattactttctaatgccacagatgcagattacatgtagttattgagaatcctttcgaattcagtggcttaatcatgaatgtctaaatattgttgacattaggatgatacatgtaaattaaagttacatttgtttagcatagacaagcttaacattgtagatgtttctcttcaaaaatcatcttaaacatttgcatttggaattgtgttaaatagaatgtgtgaaacactgtattagtaaacttcatcacctttctacttccttatagtttgaacttttcagtttttgtagttcccaaacagttgctcaatttagagcaaattaatttaacacctgccaaaaaaaggctgctgttggcttatcagttgtctttaaattcaaatgctcatgtgacttttatcacatcaaaaaatatttcattaatgattcacctttagctctgaaaattaccgcgtttagtaattatagtgggcttataaaaacatgcaactctttttgatagttatttgagaattttggtgaaaaatatttagctgagggcagtatagaacttataaaccaatatattgatatttttaaaacatttttacatataagtaaactgccatctttgagcataactacatttaaaaataaagctgcatatttttaaatcaagtgtttaacaagaatttatattttttattttttaaaattaaaaataatttatatttcctctgttgcatgaggattctcatctgtgcttataatggttagagattttatttgtgtggaatgaagtgaggcttgtagtcatggttctagtgtttcagtttgccaagtctgtttactgcagtgaaattcatcaaatgtttcagtgtggttttctgtagcctatcatttactggctatttttttatgtacacctttaggattttctgcctactctatccagttgtccaaatgatatcctacattttacaaatgccctttcagtttctattttctttttccattaaattgccctcatgtcctaatgtgcagtttgtaagtgtgtgtgtgtgtgtctgtgtgtgtgtgaatttgattttcaagagtgctagacttccaatttgagagattaaataatttaattcaggcaaacatttttcattggaatttcacagttcattgtaatgaaaatgttaatcctggatgacctttgacatacagtaatgaatcttggatattaatgaatttgttagtagcatcttgatgtgtgttttaatgagttattttcaaagttgtgcattaaaccaaagttggcatactggaagtgtttatatcaagttccatttggctactgatggacaaaaaatagaaatgccttcctatggagagtatttttcctttaaaaaattaaaaaggttaattattttgactaaaaaaaaaaaaaaaaaa

SEQ ID NO: 3 - protein, 900 aa, Homo sapiens
MATCAVEVFGLLEDEENSRIVRVRVIAGIGLAKKDILGASDPYVRVTLYDPMNGVLTSVQTKTIKKSLNPKWNEEILFRVHPQQHRLLFEVFDENRLTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 4 - DNA, 7240 bp, Homo sapiens
gctgcagctgagctcagcttaaaggtcgctggaaatcaagtgctttgtaagtgaaggcattttgactacaagcgattcaagacattttggaaacgtcagtctctggaatgggtcgtctttttctttctatgatatctggtatctaatgttttacttaggataaagactactacttaatagctcttttaggcaccacaaatgattttttcttttgatgcagtgcagctgcttctagcctgtctacggacagagatctttgttatgactttcagcccttaacatgtttggaaatgagaacaaatggcacaaagcttacgattgcactttgcagccagaagaagcaatacttaccctttgtcagaaacctccggagatgacttggatagccatgttcacatgtgcttcaaaagaccaacacggatttcaacgtctaacgttgttcaaatgaagctgactcccagacagactgcactagctccgttaataaaggaaaacgttcagtctcaagaaagatcatctgttccctcatctgaaaatgttaataaaaagagcagctgtctacagatttcactacagccaacaaggtacagtggatatcttcagtctagcaatgtcttagctgatagtgatgatgcttcgtttacttgtatcttgaaggatggtatttacagtagtgctgtggtcgataatgaattgaatgctgtgaatgatggtcaccttgtaagcagtccagccatttgtagtggtagccttagtaacttttcaaccagtgataatgggtcttacagcagcaacggtagtgattttgggtcatgtgcaagtatcacaagtggaggttcatatactaacagtgtcatcagtgacagtagtagttatacttttccaccaagtgatgatacttttttgggtggaaacttaccttctgacagcacctccaatagaagtgtgccaaacaggaatactactccttgtgaaattttttcaagaagtacaagtacagatccttttgtccaggatgacttggaacatggattagagattatgaaattgccagtgagcaggaacacaaaaattccactaaaacgttactcctccttagtcatttttcctaggagtccttcaactacccgaccgacttctccaacaagtctgtgtactcttctgagcaaaggatcctatcaaacttcacaccagtttattatttctcctagtgaaattgcacataatgaggatggcactagtgctaaaggatttctttcaacagctgtcaatggacttcggttatctaaaacaatttgtacccccggagaagtaagagacatacggccgcttcacaggaagggctcgttacagaagaaaattgttctttcgaataatactcccagacagactgtctgtgaaaagtcatctgaaggatattcttgtgtttcagtgcatttcacccaacgaaaagcagctacattagactgtgaaacaacaaatggtgattgtaaaccagaaatgtcagaaattaagcttaattctgattcagagtatattaagctcatgcataggacatctgcatgtttgccatcctcccaaaatgtagattgtcaaataaatatcaatggagaattggaaagaccacattcacagatgaacaaaaaccatggtattttacgaagaagtatttcattgggaggagcttatccaaatatttcctgtctatccagccttaagcacaattgttctaaagggggaccatctcagttactcataaagtttgcatctggaaatgaaggtaaagtggataatttatcaagagacagcaacagagattgcacaaatgaactgtctaattcttgcaagacaagagatgatttcctaggtcaagtggatgttccactttatccattaccgacagaaaatccaagattggagagaccatatacatttaaggattttgttcttcatccaagaagtcacaaatcaagagttaaaggttatctgagactaaaaatgacttatttacctaaaaccagtggctcagaagatgataatgcagaacaggctgaggaattagagcctggctgggttgttttggaccaaccagatgctgcttgccatttgcagcaacaacaagaaccttctcctctacctccagggtgggaagagaggcaggatatccttggaaggacctattatgtaaaccatgaatctagaagaacacagtggaaaagaccaacccctcaggacaacctaacagatgctgagaatggcaacattcaactgcaagcacaacgtgcatttaccaccaggcggcagatatccgaggaaacagaaagtgttgacaaccgagagtcttccgagaactgggaaattataagagaagatgaagccaccatgtatagcaaccaggccttcccatcacctccaccgtcaagtaacttggatgttccaactcatcttgcagaagaattgaatgccagactcaccatttttggaaattcagccgtgagccagccagcatcgagctcaaatcattccagcagaagaggcagcttacaagcctatacttttgaggaacaacctacacttcctgtgcttttgcctacttcatctggattaccaccaggttgggaagaaaaacaagatgaaagaggaagatcatattatgtagatcacaattccagaacgactacttggacaaagcccactgtacaggccacagtggagaccagtcagctgacctcaagccagagttctgcaggccctcaatcacaagcctccaccagtgattcaggccagcaggtgacccagccatctgaaattgagcaaggattccttcctaaaggctgggaagtccggcatgcaccaaatgggaggcctttctttattgaccacaacactaaaaccaccacctgggaagatccaagattgaaaattccagcccatctgagaggaaagacatcacttgatacttccaatgatctagggcctttacctccaggatgggaagagagaactcacacagatggaagaatcttctacataaatcacaatataaaaagaacacaatgggaagatcctcggttggagaatgtagcaataactggaccagcagtgccctactccagggattacaaaagaaagtatgagttcttccgaagaaagttgaagaagcagaatgacattccaaacaaatttgaaatgaaacttcgccgagcaactgttcttgaagactcttaccggagaattatgggtgtcaagagagcagacttcctgaaggctcgactgtggattgagtttgatggtgaaaagggattggattatggaggagttgccagagaatggttcttcctgatctcaaaggaaatgtttaacccttattatgggttgtttgaatattctgctacggacaattataccctacagataaatccaaactctggattgtgtaacgaagatcacctctcttacttcaagtttattggtcgggtagctggaatggcagtttatcatggcaaactgttggatggttttttcatccgcccattttacaagatgatgcttcacaaaccaataacccttcatgatatggaatctgtggatagtgaatattacaattccctaagatggattcttgaaaatgacccaacagaattggacctcaggtttatcatagatgaagaactttttggacagacacatcaacatgagctgaaaaatggtggatcagaaatagttgtcaccaataagaacaaaaaggaatatatttatcttgtaatacaatggcgatttgtaaaccgaatccagaagcaaatggctgcttttaaagagggattctttgaactaataccacaggatctcatcaaaatttttgatgaaaatgaactagagcttcttatgtgtggactgggagatgttgatgtgaatgactggagggaacatacaaagtataaaaatggctacagtgcaaatcatcaggttatacagtggttttggaaggctgttttaatgatggattcagaaaaaagaataagattacttcagtttgtcactggcacatctcgggtgcctatgaatggatttgctgaactatacggttcaaatggaccacagtcatttacagttgaacagtggggtactcctgaaaagctgccaagagctcatacctgttttaatcgcctggacttgccaccttatgaatcatttgaagaattatgggataaacttcagatggcaattgaaaacacccagggctttgatggagttgattagattacaaataacaatctgtagtgtttttactgccatagttttataaccaaaatcttgacttaaaattttccggggaactactaaaatgtggccactgagtcttcccagatcttgaagaaaatcatataaaaagcatttgaagaaatagtacgacaacttatttttaatcacttttaaataatgtgttgcatttacacagttgtttcatgctgtctttagagttaggtgcctgcctaaagccaggcaccaccacacctggctttagagttcacacaataggatataagtcctgtatgacttaaatagtgaattttgtccttaacatttacctcttgtatagtatctgccaggcagttttttcttaaactactgagatgataactgtgaaatatttgtgatacgtgtcatgtgtgaaaagtttgatgcattttgagatggaaaactgaaatttggaaaaagaaatactttactattgagtaaactacaatatatttagtgctactcgcagctatttattattttgtagacctgccttatgcaccttactgcctagatttttgggaaaaaactttggaaagtgtgttacctatatttctagccaactaactcacagaaaaactgtttacttcttcactttcgaagtatttggcttttgttaatatgcagttttactaaacagatggttcataagacatgtgaagcaaattcatatttgcaatgggtaaaaagtattaaagcctttctcttgcctgcatatcctattgaccattggtatagtcatcactttttcatattttagtgtagttagaagaattccttcttcaaacattaaagatccacaaagcagtatttctaaatatgccttgaagaactaaatgaagtgtatagcacttgcctttactagatattctttacacttgtacaattatgtagtaaatgtatgtttacagggtttatcatgtttacagattaagctaatttctgtagtcgcatttttatatttttagtatcactctagtaaaaaaaccaaataatttgtttaaaataaccaaagagttgttataatgcataatttgtattaaatttattactatttcttatgccttttaaaatactgtttactatgaagacaatgtttttaattacaaatccagaattctgtaggcaaaatgctacagttcatatcttcctttaaccaaactgaagtacataaagaccatgtacatgtattcatcaaacgtttattgaatgctgcgtgcctagcgctgtgctatgctctggggtaagagttgtcagcttcagagaagctgagtcctgatcctcaaggaacttgcaaatgtgtctatgaatttgtaaaacaatcaaaagtaggcgtaagcagaataaggcaaaagggaaagtgttctaggttccagcacacctgcaaagataaagtgtgccaagactgtatttatatttcatacttatattgtttcatccttatattggaatgattatatagaaaatgctcttaaaaagattaaacctatttctcagtatggtatcttggtgatttaggaataattgtaaatatatgttacgaatcttcttaaatatatatatatacacaccctgtgagaactgtaaaaagtacctctggttcttggtttaagtttgttggggtataacatgatgagtactcattagcacctgatagaaatctgaaatgtgacagtagcaaaaccactttctactttccaaacaccacagcatcagcatggtttaggggaagcaattcacagattaatgtaccctgcgttttgtcttcaccattgtcaaccagcagtcaaggatgagcaccaggtatagtcctggtttgatatctcaccagccatgtgaactgaagcagcttacttaacctctctggattcatatttcttcatctgtcaaatgagagtaatcatgcccaccctcattgtcatgccagattgtcagaagttccagacagatgagaaagcaaaaagtactttgtaaagtgttatacaatcagaaggtgctttgtgctgaccgtcagattgtgtcaagtcaggtgtgcaaattgaccacactcccaaagaatcatttaaaagtaaagtgacttctaagagacaaggaagtaggaacatttctgatattaaatatataaataatatggatacacatgtgtatatctgtatatggatcttaccatcataattcattttcttcatatcagcaaagtaatagtaatgtgggccaatgcattttggaaatgtcctcattatgtagaatggaatgtgaaaattatttttgttaaaacaggattttgccacaattatttaaatattatgtttgtgaactatctaagcatgagaaaatacaaagctttctttgtatctggctgatactcatttggatgattctgtgactcatagaacatgatgttaaatgagaccagactcaactgccaccagtccccagctgcagaccttcatccccttcatctcccactaggggctcgtggtctggaagaaacatctatcaagcaacacagccccttatcccagatagaaaagtgtctcaaatgcattctactgcgggacagtcagtaggatcattttcataaagcaaggacatcatatgtttctagaaattacaagcatataactttagcctacaatctcttattaaaaatttttaacaaaattgtattacaaatgcatttcatcagaactcaaatttaaatggtgtttgttttgggtttttatttataatgctgaagttattccatataagtatcaagttaaacacaattcattttgattataaactatttgactttttaaaatcttctgacacagtaaatatatatatcaagattgatgtatcaaaatttattgcacactttaaagtgtaaaatcattttttaaaatcttgaatccacaaataaagttctattctgattttaaaaaacaaaacaaaa

SEQ ID NO: 5 - protein, 922 aa, Homo sapiens
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 6 - DNA, 4120 bp, Homo sapiens
ggctgctggcggcctgggctgccggggccgacgcctgggtggctgctgccgccgcgcctgctgcgagatggcgatcttgggcgcggaagggtgagggcgcccgccgcaggaggaggtgccgctgccgtggccgcccggctgccgggagccgacagcttcgcgccggggttgtctcctcacagactatgagctccttgaaagagggaatcgtgtcttactcatctttgtatccccagtgtctagcagttcctgatacatagttttagctgaattttgggacatggccactgcttcaccaaggtctgatactagtaataaccacagtggaaggttgcagttacaggtaactgtttctagtgccaaacttaaaagaaaaaagaactggttcggaacagcaatatatacagaagtagttgtagatggagaaattacgaaaacagcaaaatccagtagttcttctaatccaaaatgggatgaacagctaactgtaaatgttacgccacagactacattggaatttcaagtttggagccatcgcactttaaaagcagatgctttattaggaaaagcaacgatagatttgaaacaagctctgttgatacacaatagaaaattggaaagagtgaaagaacaattaaaactttccttggaaaacaagaatggcatagcacaaactggtgaattgacagttgtgcttgatggattggtgattgagcaagaaaatataacaaactgcagctcatctccaaccatagaaatacaggaaaatggtgatgccttacatgaaaatggagagccttcagcaaggacaactgccaggttggctgttgaaggcacgaatggaatagataatcatgtacctacaagcactctagtccaaaactcatgctgctcgtatgtagttaatggagacaacacaccttcatctccgtctcaggttgctgccagacccaaaaatacaccagctccaaaaccactcgcatctgagcctgccgatgacactgttaatggagaatcatcctcatttgcaccaactgataatgcgtctgtcacgggtactccagtagtgtctgaagaaaatgccttgtctccaaattgcactagtactactgttgaagatcctccagttcaagaaatactgacttcctcagaaaacaatgaatgtattccttctaccagtgcagaattggaatctgaagctagaagtatattagagcctgacacctctaattctagaagtagttctgcttttgaagcagccaaatcaagacagccagatgggtgtatggatcctgtacggcagcagtctgggaatgccaacacagaaaccttgccatcagggtgggaacaaagaaaagatcctcatggtagaacctattatgtggatcataatactcgaactaccacatgggagagaccacaacctttacctccaggttgggaaagaagagttgatgatcgtagaagagtttattatgtggatcataacaccagaacaacaacgtggcagcggcctaccatggaatctgtccgaaattttgaacagtggcaatctcagcggaaccaattgcagggagctatgcaacagtttaaccaacgatacctctattcggcttcaatgttagctgcagaaaatgacccttatggacctttgccaccaggctgggaaaaaagagtggattcaacagacagggtttactttgtgaatcataacacaaaaacaacccagtgggaagatccaagaactcaaggcttacagaatgaagaacccctgccagaaggctgggaaattagatatactcgtgaaggtgtaaggtactttgttgatcataacacaagaacaacaacattcaaagatcctcgcaatgggaagtcatctgtaactaaaggtggtccacaaattgcttatgaacgcggctttaggtggaagcttgctcacttccgttatttgtgccagtctaatgcactacctagtcatgtaaagatcaatgtgtcccggcagacattgtttgaagattccttccaacagattatggcattaaaaccctatgacttgaggaggcgcttatatgtaatatttagaggagaagaaggacttgattatggtggcctagcgagagaatggtttttcttgctttcacatgaagttttgaacccaatgtattgcttatttgagtatgcgggcaagaacaactattgtctgcagataaatccagcatcaaccattaatccagaccatctttcatacttctgtttcattggtcgttttattgccatggcactatttcatggaaagtttatcgatactggtttctctttaccattctacaagcgtatgttaagtaaaaaacttactattaaggatttggaatctattgatactgaattttataactcccttatctggataagagataacaacattgaagaatgtggcttagaaatgtacttttctgttgacatggagattttgggaaaagttacttcacatgacctgaagttgggaggttccaatattctggtgactgaggagaacaaagatgaatatattggtttaatgacagaatggcgtttttctcgaggagtacaagaacagaccaaagctttccttgatggttttaatgaagttgttcctcttcagtggctacagtacttcgatgaaaaagaattagaggttatgttgtgtggcatgcaggaggttgacttggcagattggcagagaaatactgtttatcgacattatacaagaaacagcaagcaaatcatttggttttggcagtttgtgaaagagacagacaatgaagtaagaatgcgactattgcagttcgtcactggaacctgccgtttacctctaggaggatttgctgagctcatgggaagtaatgggcctcaaaagttttgcattgaaaaagttggcaaagacacttggttaccaagaagccatacatgttttaatcgcttggatctaccaccatataagagttatgaacaactaaaggaaaaacttctttttgcaatagaagagacagagggatttggacaagaatgaatgtggcttcttattttggaggagctcttgcatttaaataccccagccaagaaaaattgcacagatagtgtatataagctgttcattctgtacagtgaattttccgaacctctcaaagtatgttttccgttcttccacagaaatatgcaaaacagttcatccttttctactttatttattgttcccttgaaatgactgaccaggaaaaagatcatccttaaattttgaagcaagtgagagactttattaaaaatacatatatatctatataaacatatatgatagtggctctagttttatagagctccaagtgtattaaacatgacagccattcattcataaagatctggatttgctttaccttgttaatattatctaggggaaaaagtgcaaattgctccatgttcttctctcccttatgtaacatctcctgagggtgtttagttgcatggctgttcagaaaggtattaagggcttaggccaaatcttactttgagtatgttaaaaaaaaaaaaatgctgctggcttttctgaagacaggtgcttgaacttgtcagtttgttttaaataaatacaatagttgaaaatttttctctgttacatcagtaatattgttaaagtaatggatagaaccataacttacacatgaaagtcatatactagatccaatactatttagtttattatcgaaattggaaggattcattgagcagcatagaagtttgtttacatgttactttgagatgctaggtatttgtggaattaaaaagaatcaggctcttttgtactttgtttttaaatctgtgatgcttttcaaatttaattcataataaattgatgcaatttcatacttaggaacatacaaaaggtaatgtaaactctgccacttttttgtgttcaaaattttggtttttatgaagccagatggattgaagagttacataagcatttgaatgctctaatataaggctaatgattttctgttagtgtttgaatatcttcattcctctcaaattcataacagttctatttaactgaattaaataaccatatgaaaaaaaaaaaaaaaaa

SEQ ID NO: 7 - DNA, 19 bp, Artificial Sequence
  Description of Artificial Sequence Synthetic WWP1 promoter forward primer
gtccggagttggaggcttt

SEQ ID NO: 8 - DNA, 18 bp, Artificial Sequence
  Description of Artificial Sequence Synthetic WWP1 promoter reverse primer
gaccccacacctcccttc

SEQ ID NO: 9 - DNA, 18 bp, Artificial Sequence
  Description of Artificial Sequence Synthetic NEDD4-1 promoter forward primer
ccgtcaaccacccacctc

SEQ ID NO: 10 - DNA, 18 bp, Artificial Sequence
  Description of Artificial Sequence Synthetic NEDD4-1 promoter reverse primer
ctccctcagcgacagcag

SEQ ID NO: 11 - DNA, 23 bp, Artificial Sequence
  Description of Artificial Sequence Synthetic JunB forward primer
aagcccacagagagaggtggaag

SEQ ID NO: 12 - DNA, 24 bp, Artificial Sequence
  Description of Artificial Sequence Synthetic JunB reverse primer
ccagaaggtggtgcctttttattg

SEQ ID NO: 13 - DNA, 25 bp, Artificial Sequence
  Description of Artificial Sequence Synthetic WWP1 sgRNA forward primer
caccgatcagctgctcgtcccattt

SEQ ID NO: 14 - DNA, 25 bp, Artificial Sequence
  Description of Artificial Sequence Synthetic WWP1 sgRNA reverse primer
aaacaaatgggacgagcagctgatc

SEQ ID NO: 15 - DNA, 25 bp, Artificial Sequence
  Description of Artificial Sequence Synthetic WWP1 sgRNA forward primer
caccgtaatactcgaactactacat

SEQ ID NO: 16 - DNA, 25 bp, Artificial Sequence
  Description of Artificial Sequence Synthetic WWP1 sgRNA reverse primer
aaacatgtagtagttcgagtattac

SEQ ID NO: 17 - DNA, 22 bp, Artificial Sequence
  Description of Artificial Sequence Synthetic WWP1 forward primer
tgcttcaccaaggtctgatact

SEQ ID NO: 18 - DNA, 22 bp, Artificial Sequence
  Description of Artificial Sequence Synthetic WWP1 reverse primer
gctgttccgaaccagttctttt

SEQ ID NO: 19 - DNA, 19 bp, Artificial Sequence
  Description of Artificial Sequence Synthetic Trim27 forward primer
agcccatgatgctcgactg

SEQ ID NO: 20 - DNA, 19 bp, Artificial Sequence
  Description of Artificial Sequence Synthetic Trim27 reverse primer
gggcacgacacgttagtct

SEQ ID NO: 21 - DNA, 21 bp, Artificial Sequence
  Description of Artificial Sequence Synthetic Itch forward primer
tgatgatggctccagatccaa

SEQ ID NO: 22 - DNA, 23 bp, Artificial Sequence
  Description of Artificial Sequence Synthetic Itch reverse primer
gactctcctattttcaccagctc

SEQ ID NO: 23 - DNA, 21 bp, Artificial Sequence
  Description of Artificial Sequence Synthetic RNF168 forward primer
ggatctgcatggaaatcctcg

SEQ ID NO: 24 - DNA, 21 bp, Artificial Sequence
  Description of Artificial Sequence Synthetic RNF168 reverse primer
actggaagcacggtttacaca

SEQ ID NO: 25 - DNA, 20 bp, Artificial Sequence
  Description of Artificial Sequence Synthetic Actin forward primer
ctcttccagccttccttcct

SEQ ID NO: 26 - DNA, 20 bp, Artificial Sequence
  Description of Artificial Sequence Synthetic Actin reverse primer
agcactgtgttggcgtacag

SEQ ID NO: 27 - protein, 4 aa, Artificial Sequence
  Description of Artificial Sequence Synthetic peptide
LRGG

SEQ ID NO: 28 - protein, 18 aa, Artificial Sequence
  Description of Artificial Sequence Synthetic peptide
TITLEVEPSDTIENVKAK

SEQ ID NO: 29 - protein, 9 aa, Artificial Sequence
  Description of Artificial Sequence Synthetic peptide
YFSPNFKVK

SEQ ID NO: 30 - protein, 7 aa, Artificial Sequence
  Description of Artificial Sequence Synthetic peptide
VKLYFTK

SEQ ID NO: 31 - protein, 27 aa, Homo sapiens
LTKNDLDKANKDKANRYFSPNFKVKLY

SEQ ID NO: 32 - protein, 27 aa, Macaca mulatta
LTKNDLDKANKDKANRYFSPNFKVKLY

SEQ ID NO: 33 - protein, 27 aa, Mus musculus
LTKNDLDKANKDKANRYFSPNFKVKLY

SEQ ID NO: 34 - protein, 27 aa, Tupaia chinensis
LTKNDLDKANKDKANRYFSPNFKVKLY

SEQ ID NO: 35 - protein, 27 aa, Xenopus laevis
LTKNDLDKANKDKANRYFSPNFKVKLY

SEQ ID NO: 36 - protein, 27 aa, Takifugu rubripes
LSKNDRDKANKDKANRYFSPNFKVKLY

SEQ ID NO: 37 - protein, 27 aa, Saccoglossus kowalevskii
LRKDEIDKANKDKANRYFSPNFKVKLY

(19) United States
(12) Patent Application Publication
(10) Pub. No.: US 2019/0374506 A1
(43) Pub. Date: Dec. 12, 2019

(54) COMPOSITIONS AND METHODS FOR TREATING CANCER

(71) Applicant: BETH ISREAL DEACONESS MEDICAL CENTER, BOSTON, MA (US)

(72) Inventors: YU-RU LEE, BOSTON, MA (US); PIER PAOLO PANDOLFI, BOSTON, MA (US)

(73) Assignee: BETH ISREAL DEACONESS MEDICAL CENTER, BOSTON, MA (US)

(21) Appl. No.: 15/739,938

(22) PCT Filed: Aug. 29, 2017
(86) PCT No.: PCT/US17/49098

§ 371 Date: Feb. 16, 2018

Related U.S. Application Data
(60) Provisional application No. 62/381,294, filed on Aug. 30, 2016.

(51) Int. Cl. A61K 31/404; A61P 35/00

(52) U.S. Cl. CPC A61K 31/404; A61P 35/00

(57) ABSTRACT

In some embodiments, the present invention provides a method of treating cancer, the method comprising administering to a subject having a cancer, an effective amount an agent that inhibits the expression or activity of neural precursor cell expressed developmentally down-regulated protein 4 (NEDD4), and an agent that inhibits the expression or activity of WW domain-containing protein-1 (WWP1).

### CROSS-REFERENCE TO RELATED APPLICATIONS

This application claims the benefit of and priority to U.S. Provisional Patent Application Ser. No. 62/381,294, filed Aug. 30, 2016, which is incorporated herein by reference in its entirety.

### STATEMENT OF RIGHTS TO INVENTIONS MADE UNDER FEDERALLY SPONSORED RESEARCH

This invention was made with government support under Grant No. CA082328 awarded by the National Institutes of Health. The government has certain rights in the invention.

### BACKGROUND OF THE INVENTION

Phosphatase and tensin homolog deleted on chromosome ten (PTEN) is a lipid and protein dual phosphatase, and is a tumor suppressor. PTEN is frequently mutated, deleted, or epigenetically silenced in various types of human cancers. In the cytoplasm, PTEN primarily governs key cellular processes including cell survival, proliferation, aging, angiogenesis and metabolism through its lipid phosphatase activity to antagonize the PI3K-Akt oncogenic pathway. PTEN is active as a dimer within membrane compartments. However, the mechanisms regulating PTEN dimerization remain to be fully characterized.

Ubiquitination covalently attaches the 76 amino-acid ubiquitin polypeptide to the lysine residues of a target protein. This covalent modification represents one of the most abundant and important post-translational protein modifications in mammalian cells. The seven lysine residues and the Met-1 residue in a ubiquitin molecule can be utilized to mediate the conjugation of another ubiquitin moiety, leading to the formation of poly-ubiquitin chains with various lengths and linkages on substrates. These topologically distinct polymers can affect diverse biological functions, making ubiquitination one of the most versatile post-translational modifications in cells. For example, numerous studies have shown the essential roles of K48-linked chains in proteasomal degradation, whereas K63-linked chains function as a platform for protein-protein interaction important in various signaling pathways. Emerging evidence has shown that K11-linked ubiquitin chains also target substrates for proteasomal degradation, while linear Met1-linked chains serve as a non-degradable signal and function in immune and NFκB pathways. In contrast, little is known about the functions of the remaining atypical ubiquitin chain types, including the chain type linked through K27.

PTEN is one of the most frequently mutated, deleted, or silenced tumor suppressor genes in human cancer. Functionally, PTEN encodes a dual specificity phosphatase whose major substrate is phosphatidylinositol 3, 4, 5 trisphosphate (PIP₃). PTEN dephosphorylates the D3-phosphate of the second messenger PIP₃ and opposes the activation of the proto-oncogenic PI3K/AKT signaling pathway, thus controlling cell proliferation, cell growth and cell metabolism.

MYC is a critical transcription factor involved in multiple biological processes, including replication, cell division, protein synthesis and metabolism. Frequent alterations in chromosome 8q24 in the region of MYC, leading to the amplification of MYC, have been linked to disease aggressiveness. A number of studies in cancers of diverse histological origin have indicated that MYC appears to be pervasively activated during tumor progression, such as prostate and breast cancer. Many of its pro-tumorigenic functions have been attributed to its ability to aberrantly activate the expression of downstream target genes. Furthermore, activation of the PI3K-AKT signaling pathway and MYC amplification are frequently found to co-occur in cancers and correlate with a high histological grade and a poor prognosis. However, the underlying mechanisms of how MYC crosstalks with PI3K-AKT pathway activation remain elusive.

PTEN is strictly regulated, and deregulation of its function through aberrant subcellular localization and post-translational modifications are key events in tumorigenesis. Mechanistically, mono-ubiquitination regulates PTEN nuclear compartmentalization, where it exerts PIP3 independent functions. PTEN may also exist as a dimer, and that dimer formation and membrane recruitment are crucial for PTEN function and activation. However, the mechanisms that regulate PTEN dimerization and favor membrane recruitment remain largely unknown. The elucidation of such mechanisms is expected to identify new cancer therapies, which are urgently required.

### SUMMARY OF THE INVENTION

The invention generally provides a method of treating cancer, the method comprising administering to a subject having a cancer, an effective amount of an agent that inhibits the expression or activity of neural precursor cell expressed developmentally down-regulated protein 4 (NEDD4-1), and an agent that inhibits the expression or activity of WW domain-containing protein-1 (WWP1).

In one aspect, the present invention features a method of treating cancer in a selected subject, the method comprising administering to the subject an effective amount of an agent that inhibits the expression or activity of NEDD4-1 or WWP1, wherein the subject is selected by a method comprising detecting increased expression in MYC, NEDD4-1 or WWP1 relative to a reference.

In another aspect, the present invention features a method of treating cancer in a subject, the method comprising administering to the subject an effective amount of an agent that inhibits the expression or activity of NEDD4-1 and an agent that inhibits the expression or activity of WW domain-containing protein-1 (WWP1).

In another aspect, the present invention features a method of inhibiting neural precursor cell expressed developmentally down-regulated protein 4 (NEDD4-1) and WW domain-containing protein-1 (WWP1) in a neoplastic cell, the method comprising contacting the cell with an agent that inhibits NEDD4-1 expression or activity and an agent that inhibits WWP1 expression or activity.

In another aspect, the present invention features a method of inhibiting the survival or proliferation of a neoplastic cell having increased MYC expression, the method comprising contacting the cell with an agent that inhibits NEDD4-1 and an agent that inhibits WWP1 expression or activity, wherein the cell is characterized as having increased MYC expression, thereby inhibiting the survival or proliferation of the neoplastic cell.

In various embodiments of any of the above aspects or any other aspect of the invention delineated herein, the neoplastic cell is a mammalian cell. In other embodiments, the mammalian cell is a murine, rat, or human cell. In still other embodiments, the cell is in vitro or in vivo. In still other embodiments, the neoplastic cell or cancer comprises a mutation in PTEN. In still other embodiments, the neoplastic cell or cancer overexpresses cMYC. In still other embodiments, the method reduces neoplastic cell survival or proliferation. In still other embodiments, the neoplastic cell is derived from prostate cancer, breast cancer, or colorectal cancer. In still other embodiments, the subject has prostate cancer, breast cancer, or colorectal cancer. In still other embodiments, the agent is a polypeptide, polynucleotide, or a small molecule. In still other embodiments, the polynucleotide is an inhibitory nucleic acid molecule that inhibits the expression of NEDD4-1 or WWP1. In still other embodiments, the inhibitory nucleic acid molecule is an antisense molecule, siRNA, or shRNA. In still other embodiments, the agent is selected from the group consisting of: 4-(4-chlorobenzoyl) piperazin-1-yl) (4-(phonoxymethyl) phenyl) methanone, and indole-3-carbinol. In still other embodiments, the agent inhibits the formation of a NEDD4-1/WWP1 heterodimer. In still other embodiments, the method comprising detecting an alteration in a marker selected from the group consisting of PTEN, MYC, WWP1, and NEDD4-1, wherein detection of said alteration indicates that the subject should be treated with an agent that inhibits WWP1 and/or NEDD4-1 expression or activity. In still other embodiments, the alteration in PTEN is a mutation that reduces PTEN expression or activity. In still other embodiments, the alteration in MYC results in MYC amplification or overexpression. In still other embodiments, the alteration in WWP1 and NEDD4-1 results in WWP1 or NEDD4-1 overexpression. In still other embodiments, overexpression of MYC, NEDD4-1, and WWP1 is detected. In still other embodiments, the agent is a polypeptide, polynucleotide, or a small molecule. In still other embodiments, the polynucleotide is an inhibitory nucleic acid molecule that inhibits the expression of NEDD4 or WWP1. In still other embodiments, the inhibitory nucleic acid molecule is an antisense molecule, siRNA, or shRNA. In still other embodiments, the agent is selected from the group consisting of: 4-(4-chlorobenzoyl) piperazin-1-yl) (4-(phonoxymethyl) phenyl) methanone, and indole-3-carbinol.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Phosphatase And Tensin Homolog Deleted On Chromosome Ten (PTEN) polypeptide” is meant a protein having at least about 85% amino acid identity to the sequence provided at NCBI Reference Sequence: NP 002985.1, or a fragment thereof, and having phosphatase activity. Examples of PTEN proteins include the human PTEN protein having the sequence listed in the NCBI reference sequence NP 000305.3, the sequence of which is provided herein below (SEQ ID NO: 1):

| | |
|---|---|
| 1 | mtaiikeivs rnkrryqedg fdldltyiyp niiamgfpae rlegvyrnni ddvvrfldsk |
| 61 | hknhykiynl caerhydtak fncrvaqypf edhnppqlel ikpfcedldq wlseddnhva |
| 121 | aihckagkgr tgvmicayll hrgkflkaqe aldfygevrt rdkkgvtips qrryvyyysy |
| 181 | llknhldyrp vallfhkmmf etipmfsggt cnpqfvvcql kvkiyssnsg ptrredkfmy |
| 241 | fefpqplpvc gdikveffhk qnkmlkkdkm fhfwvntffi pgpeetsekv engslcdqei |
| 301 | dsicsierad ndkeylvltl tkndldkank dkanryfspn fkvklyftkt veepsnpeas |
| 361 | sstsvtpdvs dnepdhyrys dttdsdpene pfdedqhtqi tkv |

By “PTEN polynucleotide” is meant a nucleic acid molecule encoding a PTEN polypeptide. An exemplary PTEN polynucleotide sequence is provided at NCBI Reference Sequence: NM_000314.6, and reproduced herein below (SEQ ID NO: 2).

| | |
|---|---|
| 1 | cctcccctcg cccggcgcgg tcccgtccgc ctctcgctcg cctcccgcct cccctcggtc |
| 61 | ttccgaggcg cccgggctcc cggcgcggcg gcggaggggg cgggcaggcc ggcgggcggt |
| 121 | gatgtggcgg gactctttat gcgctgcggc aggatacgcg ctcggcgctg ggacgcgact |
| 181 | gcgctcagtt ctctcctctc ggaagctgca gccatgatgg aagtttgaga gttgagccgc |
| 241 | tgtgaggcga ggccgggctc aggcgaggga gatgagagac ggcggcggcc gcggcccgga |
| 301 | gcccctctca gcgcctgtga gcagccgcgg gggcagcgcc ctcggggagc cggccggcct |
| 361 | gcggcggcgg cagcggcggc gtttctcgcc tcctcttcgt cttttctaac cgtgcagcct |
| 421 | cttcctcggc ttctcctgaa agggaaggtg gaagccgtgg gctcgggcgg gagccggctg |
| 481 | aggcgcggcg gcggcggcgg cacctcccgc tcctggagcg ggggggagaa gcggcggcgg |
| 541 | cggcggccgc ggcggctgca gctccaggga gggggtctga gtcgcctgtc accatttcca |
| 601 | gggctgggaa cgccggagag ttggtctctc cccttctact gcctccaaca cggcggcggc |
| 661 | ggcggcggca catccaggga cccgggccgg ttttaaacct cccgtccgcc gccgccgcac |
| 721 | cccccgtggc ccgggctccg gaggccgccg gcggaggcag ccgttcggag gattattcgt |
| 781 | cttctcccca ttccgctgcc gccgctgcca ggcctctggc tgctgaggag aagcaggccc |
| 841 | agtcgctgca accatccagc agccgccgca gcagccatta cccggctgcg gtccagagcc |
| 901 | aagcggcggc agagcgaggg gcatcagcta ccgccaagtc cagagccatt tccatcctgc |
| 961 | agaagaagcc ccgccaccag cagcttctgc catctctctc ctcctttttc ttcagccaca |
| 1021 | ggctcccaga catgacagcc atcatcaaag agatcgttag cagaaacaaa aggagatatc |
| 1081 | aagaggatgg attcgactta gacttgacct atatttatcc aaacattatt gctatgggat |
| 1141 | ttcctgcaga aagacttgaa ggcgtataca ggaacaatat tgatgatgta gtaaggtttt |
| 1201 | tggattcaaa gcataaaaac cattacaaga tatacaatct ttgtgctgaa agacattatg |
| 1261 | acaccgccaa atttaattgc agagttgcac aatatccttt tgaagaccat aacccaccac |
| 1321 | agctagaact tatcaaaccc ttttgtgaag atcttgacca atggctaagt gaagatgaca |
| 1381 | atcatgttgc agcaattcac tgtaaagctg gaaagggacg aactggtgta atgatatgtg |
| 1441 | catatttatt acatcggggc aaatttttaa aggcacaaga ggccctagat ttctatgggg |
| 1501 | aagtaaggac cagagacaaa aagggagtaa ctattcccag tcagaggcgc tatgtgtatt |
| 1561 | attatagcta cctgttaaag aatcatctgg attatagacc agtggcactg ttgtttcaca |
| 1621 | agatgatgtt tgaaactatt ccaatgttca gtggcggaac ttgcaatcct cagtttgtgg |
| 1681 | tctgccagct aaaggtgaag atatattcct ccaattcagg acccacacga cgggaagaca |
| 1741 | agttcatgta ctttgagttc cctcagccgt tacctgtgtg tggtgatatc aaagtagagt |
| 1801 | tcttccacaa acagaacaag atgctaaaaa aggacaaaat gtttcacttt tgggtaaata |
| 1861 | cattcttcat accaggacca gaggaaacct cagaaaaagt agaaaatgga agtctatgtg |
| 1921 | atcaagaaat cgatagcatt tgcagtatag agcgtgcaga taatgacaag gaatatctag |
| 1981 | tacttacttt aacaaaaaat gatcttgaca aagcaaataa agacaaagcc aaccgatact |
| 2041 | tttctccaaa ttttaaggtg aagctgtact tcacaaaaac agtagaggag ccgtcaaatc |
| 2101 | cagaggctag cagttcaact tctgtaacac cagatgttag tgacaatgaa cctgatcatt |
| 2161 | atagatattc tgacaccact gactctgatc cagagaatga accttttgat gaagatcagc |
| 2221 | atacacaaat tacaaaagtc tgaatttttt tttatcaaga gggataaaac accatgaaaa |
| 2281 | taaacttgaa taaactgaaa atggaccttt ttttttttaa tggcaatagg acattgtgtc |
| 2341 | agattaccag ttataggaac aattctcttt tcctgaccaa tcttgtttta ccctatacat |
| 2401 | ccacagggtt ttgacacttg ttgtccagtt gaaaaaaggt tgtgtagctg tgtcatgtat |
| 2461 | ataccttttt gtgtcaaaag gacatttaaa attcaattag gattaataaa gatggcactt |
| 2521 | tcccgtttta ttccagtttt ataaaaagtg gagacagact gatgtgtata cgtaggaatt |
| 2581 | ttttcctttt gtgttctgtc accaactgaa gtggctaaag agctttgtga tatactggtt |
| 2641 | cacatcctac ccctttgcac ttgtggcaac agataagttt gcagttggct aagagaggtt |
| 2701 | tccgaagggt tttgctacat tctaatgcat gtattcgggt taggggaatg gagggaatgc |
| 2761 | tcagaaagga aataatttta tgctggactc tggaccatat accatctcca gctatttaca |
| 2821 | cacacctttc tttagcatgc tacagttatt aatctggaca ttcgaggaat tggccgctgt |
| 2881 | cactgcttgt tgtttgcgca ttttttttta aagcatattg gtgctagaaa aggcagctaa |
| 2941 | aggaagtgaa tctgtattgg ggtacaggaa tgaaccttct gcaacatctt aagatccaca |
| 3001 | aatgaaggga tataaaaata atgtcatagg taagaaacac agcaacaatg acttaaccat |
| 3061 | ataaatgtgg aggctatcaa caaagaatgg gcttgaaaca ttataaaaat tgacaatgat |
| 3121 | ttattaaata tgttttctca attgtaacga cttctccatc tcctgtgtaa tcaaggccag |
| 3181 | tgctaaaatt cagatgctgt tagtacctac atcagtcaac aacttacact tattttacta |
| 3241 | gttttcaatc ataatacctg ctgtggatgc ttcatgtgct gcctgcaagc ttcttttttc |
| 3301 | tcattaaata taaaatattt tgtaatgctg cacagaaatt ttcaatttga gattctacag |
| 3361 | taagcgtttt ttttctttga agatttatga tgcacttatt caatagctgt cagccgttcc |
| 3421 | acccttttga ccttacacat tctattacaa tgaattttgc agttttgcac attttttaaa |
| 3481 | tgtcattaac tgttagggaa ttttacttga atactgaata catataatgt ttatattaaa |
| 3541 | aaggacattt gtgttaaaaa ggaaattaga gttgcagtaa actttcaatg ctgcacacaa |
| 3601 | aaaaaagaca tttgattttt cagtagaaat tgtcctacat gtgctttatt gatttgctat |
| 3661 | tgaaagaata gggttttttt tttttttttt tttttttttt ttaaatgtgc agtgttgaat |
| 3721 | catttcttca tagtgctccc ccgagttggg actagggctt caatttcact tcttaaaaaa |
| 3781 | aatcatcata tatttgatat gcccagactg catacgattt taagcggagt acaactacta |
| 3841 | ttgtaaagct aatgtgaaga tattattaaa aaggtttttt tttccagaaa tttggtgtct |
| 3901 | tcaaattata ccttcacctt gacatttgaa tatccagcca ttttgtttct taatggtata |
| 3961 | aaattccatt ttcaataact tattggtgct gaaattgttc actagctgtg gtctgaccta |
| 4021 | gttaatttac aaatacagat tgaataggac ctactagagc agcatttata gagtttgatg |
| 4081 | gcaaatagat taggcagaac ttcatctaaa atattcttag taaataatgt tgacacgttt |
| 4141 | tccatacctt gtcagtttca ttcaacaatt tttaaatttt taacaaagct cttaggattt |
| 4201 | acacatttat atttaaacat tgatatatag agtattgatt gattgctcat aagttaaatt |
| 4261 | ggtaaagtta gagacaacta ttctaacacc tcaccattga aatttatatg ccaccttgtc |
| 4321 | tttcataaaa gctgaaaatt gttacctaaa atgaaaatca acttcatgtt ttgaagatag |
| 4381 | ttataaatat tgttctttgt tacaatttcg ggcaccgcat attaaaacgt aactttattg |
| 4441 | ttccaatatg taacatggag ggccaggtca taaataatga cattataatg ggcttttgca |
| 4501 | ctgttattat ttttcctttg gaatgtgaag gtctgaatga gggttttgat tttgaatgtt |
| 4561 | tcaatgtttt tgagaagcct tgcttacatt ttatggtgta gtcattggaa atggaaaaat |
| 4621 | ggcattatat atattatata tataaatata tattatacat actctcctta ctttatttca |
| 4681 | gttaccatcc ccatagaatt tgacaagaat tgctatgact gaaaggtttt cgagtcctaa |
| 4741 | ttaaaacttt atttatggca gtattcataa ttagcctgaa atgcattctg taggtaatct |
| 4801 | ctgagtttct ggaatatttt cttagacttt ttggatgtgc agcagcttac atgtctgaag |
| 4861 | ttacttgaag gcatcacttt taagaaagct tacagttggg ccctgtacca tcccaagtcc |
| 4921 | tttgtagctc ctcttgaaca tgtttgccat acttttaaaa gggtagttga ataaatagca |
| 4981 | tcaccattct ttgctgtggc acaggttata aacttaagtg gagtttaccg gcagcatcaa |
| 5041 | atgtttcagc tttaaaaaat aaaagtaggg tacaagttta atgtttagtt ctagaaattt |
| 5101 | tgtgcaatat gttcataacg atggctgtgg ttgccacaaa gtgcctcgtt tacctttaaa |
| 5161 | tactgttaat gtgtcatgca tgcagatgga aggggtggaa ctgtgcacta aagtgggggc |
| 5221 | tttaactgta gtatttggca gagttgcctt ctacctgcca gttcaaaagt tcaacctgtt |
| 5281 | ttcatataga atatatatac taaaaaattt cagtctgtta aacagcctta ctctgattca |
| 5341 | gcctcttcag atactcttgt gctgtgcagc agtggctctg tgtgtaaatg ctatgcactg |
| 5401 | aggatacaca aaaataccaa tatgatgtgt acaggataat gcctcatccc aatcagatgt |
| 5461 | ccatttgtta ttgtgtttgt taacaaccct ttatctctta gtgttataaa ctccacttaa |
| 5521 | aactgattaa agtctcattc ttgtcattgt gtgggtgttt tattaaatga gagtttataa |
| 5581 | ttcaaattgc ttaagtccat tgaagtttta attaatgggc agccaaatgt gaatacaaag |
| 5641 | ttttcagttt ttttttttcc tgctgtcctt caaagcctac tgtttaaaaa aaaaaaaaaa |
| 5701 | aaaaaacatg gcctgagagt agagtatctg tctactcatg tttaattaag gaaaaacact |
| 5761 | tatttttagg gctttagtca tcacttcata aattgtataa gcacattaaa tagcgttcta |
| 5821 | gtcctgaaaa agtccaagat tcttagaaaa ttgtgcatat ttttattatg acagatgttt |
| 5881 | gaagataatt ccccagaatg gatttgatac tttagatttc aattttgtgg cttttgtcta |
| 5941 | ttattctgta ctctgccatc agcatatgga aagcttcatt tactcatcat gacttgtgcc |
| 6001 | atataaaaat tgatatttcg gaatagtcta aaggactttt tgtacttgaa tttaatcatg |
| 6061 | ttgtttctaa tattcttaaa agcttgaaga ctaaagcata tcctttcaac aaagcatagt |
| 6121 | aaggtaataa gaaagtgtag tttgtacaag tgttaaaaaa ataaagtaga caatgttaca |
| 6181 | gtgggactta ttatttcaag tttacatttt ctccatgtaa ttttttaaaa agtaaatgaa |
| 6241 | aaaatgtgca ataatgtaaa atatgaagtg tatgtgtaca cacattttat ttttcggtat |
| 6301 | cttgggtata cgtatggttg aaaactatac tggagtctaa aagtattcta atttataaga |
| 6361 | agacattttg gtgatgtttg aaaaatagaa atgtgctagt tttgttttta tatcatgtcc |
| 6421 | tttgtacgtt gtaatatgag ctggcttggt tcagtaaatg ccatcaccat ttccattgag |
| 6481 | aatttaaaac tcaccagtgt ttaatatgca ggcttccaaa ggcttatgaa aaaaatcaag |
| 6541 | acccttaaat ctagttaatt tgctgctaac atgaaactct ttggttcttt tatttttgcc |
| 6601 | agataattag acacacatct aaagcttagt cttaaatggc ttaagtgtag ctattgatta |
| 6661 | gtgctgttgc tagttcagaa agaaatgttt gtgaatggaa acaagaatat tcagtccaaa |
| 6721 | ctgttgtaag gacagtacct gaaaaccagg aaacaggata atggaaaaag tcttttaaag |
| 6781 | atgaaatgtt ggagccaact ttcttataga attaattgta tgtggctata gaaagcctaa |
| 6841 | tgattgttgc ttatttttga gagcatatta ttcttttatg accataatct tgctgttttt |
| 6901 | ccatcttcca aaagatcttc cttctaatat gtatatcaga atgtgggtag ccagtcagac |
| 6961 | aaattcatat tggttggtag ctttaaaaag tttgtaatgt gaagacagga aaggacaaaa |
| 7021 | tagtttgctt tggtggtagt actctggttg ttaagctagg tattttgaga ctacttcccc |
| 7081 | atcacaacaa caataaaata atcactcata atcctatcac ctggagacat agccatcgtt |
| 7141 | aatatgttag tgactataca atcatgtttt cttctgtata tccatgtata ttctttaaaa |
| 7201 | atgaaattta tactgtacct gatctcaaag ctttttagct tagtatatct gtcatgaatt |
| 7261 | tgtaggatgt tccattgcat cagaaaacgg acagtgattt gattactttc taatgccaca |
| 7321 | gatgcagatt acatgtagtt attgagaatc ctttcgaatt cagtggctta atcatgaatg |
| 7381 | tctaaatatt gttgacatta ggatgataca tgtaaattaa agttacattt gtttagcata |
| 7441 | gacaagctta acattgtaga tgtttctctt caaaaatcat cttaaacatt tgcatttgga |
| 7501 | attgtgttaa atagaatgtg tgaaacactg tattagtaaa cttcatcacc tttctacttc |
| 7561 | cttatagttt gaacttttca gtttttgtag ttcccaaaca gttgctcaat ttagagcaaa |
| 7621 | ttaatttaac acctgccaaa aaaaggctgc tgttggctta tcagttgtct ttaaattcaa |
| 7681 | atgctcatgt gacttttatc acatcaaaaa atatttcatt aatgattcac ctttagctct |
| 7741 | gaaaattacc gcgtttagta attatagtgg gcttataaaa acatgcaact ctttttgata |
| 7801 | gttatttgag aattttggtg aaaaatattt agctgagggc agtatagaac ttataaacca |
| 7861 | atatattgat atttttaaaa catttttaca tataagtaaa ctgccatctt tgagcataac |
| 7921 | tacatttaaa aataaagctg catattttta aatcaagtgt ttaacaagaa tttatatttt |
| 7981 | ttatttttta aaattaaaaa taatttatat ttcctctgtt gcatgaggat tctcatctgt |
| 8041 | gcttataatg gttagagatt ttatttgtgt ggaatgaagt gaggcttgta gtcatggttc |
| 8101 | tagtgtttca gtttgccaag tctgtttact gcagtgaaat tcatcaaatg tttcagtgtg |
| 8161 | gttttctgta gcctatcatt tactggctat ttttttatgt acacctttag gattttctgc |
| 8221 | ctactctatc cagttgtcca aatgatatcc tacattttac aaatgccctt tcagtttcta |
| 8281 | ttttcttttt ccattaaatt gccctcatgt cctaatgtgc agtttgtaag tgtgtgtgtg |
| 8341 | tgtgtctgtg tgtgtgtgaa tttgattttc aagagtgcta gacttccaat ttgagagatt |
| 8401 | aaataattta attcaggcaa acatttttca ttggaatttc acagttcatt gtaatgaaaa |
| 8461 | tgttaatcct ggatgacctt tgacatacag taatgaatct tggatattaa tgaatttgtt |
| 8521 | agtagcatct tgatgtgtgt tttaatgagt tattttcaaa gttgtgcatt aaaccaaagt |
| 8581 | tggcatactg gaagtgttta tatcaagttc catttggcta ctgatggaca aaaaatagaa |
| 8641 | atgccttcct atggagagta tttttccttt aaaaaattaa aaaggttaat tattttgact |
| 8701 | aaaaaaaaaa aaaaaaaa |

By “Neural Precursor Cell Expressed Developmentally Down-Regulated Protein 4 polypeptide (NEDD4)” or “(NEDD4-1)” is meant a protein having at least about 85% amino acid sequence identity to NCBI Reference Sequence: NP_001271267 and having E3 ubiquitin-protein ligase activity. NEDD4-1 is frequently overexpressed in cancers, such as, for example, gastric adenocarcinoma, colon adenocarcinoma, prostate cancer, bladder cancer, and breast cancer. An exemplary NEDD4-1 amino acid sequence is provided herein below (SEQ ID NO: 3):

| | |
|---|---|
| 1 | matcavevfg lledeensri vrvrviagig lakkdilgas dpyvrvtlyd pmngyltsyq |
| 61 | tktikkslnp kwneeilfrv hpqqhrllfe vfdenrltrd dflgqvdvpl yplptenprl |
| 121 | erpytfkdfv lhprshksrv kgylrlkmty lpktsgsedd naeqaeelep gwvvldqpda |
| 181 | achlqqqqep splppgweer qdilgrtyyv nhesrrtqwk rptpqdnltd aengniqlqa |
| 241 | qrafttrrqi seetesvdnr essenweiir edeatmysnq afpspppssn ldvpthlaee |
| 301 | lnarltifgn saysqpasss nhssrrgslq aytfeeqptl pvllptssgl ppgweekqde |
| 361 | rgrsyyvdhn srtttwtkpt vqatvetsql tssqssagpq sqastsdsgq qvtqpseieq |
| 421 | gflpkgwevr hapngrpffi dhntktttwe dprlkipahl rgktsldtsn dlgplppgwe |
| 481 | erthtdgrif yinhnikrtq wedprlenva itgpavpysr dykrkyeffr rklkkqndip |
| 541 | nkfemklrra tvledsyrri mgvkradflk arlwiefdge kgldyggvar ewffliskem |
| 601 | fnpyyglfey satdnytlqi npnsglcned hlsyfkfigr vagmavyhgk lldgffirpf |
| 661 | ykmmlhkpit lhdmesvdse yynslrwile ndpteldlrf iideelfgqt hqhelknggs |
| 721 | eivvtnknkk eyiylviqwr fvnriqkqma afkegffeli pqdlikifde nelellmcgl |
| 781 | gdvdvndwre htkykngysa nhqviqwfwk avlmmdsekr irllqfvtgt srvpmngfae |
| 841 | lygsngpqsf tveqwgtpek lprahtcfnr ldlppyesfe elwdklqmai entqgfdgvd |

By“NEDD4 or NEDD4-1 polynucleotide” is meant a nucleic acid molecule encoding a NEDD4-1 polypeptide. An exemplary NEDD4-1 polynucleotide sequence is provided at NCBI Reference Sequence: NM 001284338, and reproduced herein below (SEQ ID NO: 4):

| | |
|---|---|
| 1 | gctgcagctg agctcagctt aaaggtcgct ggaaatcaag tgctttgtaa gtgaaggcat |
| 61 | tttgactaca agcgattcaa gacattttgg aaacgtcagt ctctggaatg ggtcgtcttt |
| 121 | ttctttctat gatatctggt atctaatgtt ttacttagga taaagactac tacttaatag |
| 181 | ctcttttagg caccacaaat gattttttct tttgatgcag tgcagctgct tctagcctgt |
| 241 | ctacggacag agatctttgt tatgactttc agcccttaac atgtttggaa atgagaacaa |
| 301 | atggcacaaa gcttacgatt gcactttgca gccagaagaa gcaatactta ccctttgtca |
| 361 | gaaacctccg gagatgactt ggatagccat gttcacatgt gcttcaaaag accaacacgg |
| 421 | atttcaacgt ctaacgttgt tcaaatgaag ctgactccca gacagactgc actagctccg |
| 481 | ttaataaagg aaaacgttca gtctcaagaa agatcatctg ttccctcatc tgaaaatgtt |
| 541 | aataaaaaga gcagctgtct acagatttca ctacagccaa caaggtacag tggatatctt |
| 601 | cagtctagca atgtcttagc tgatagtgat gatgcttcgt ttacttgtat cttgaaggat |
| 661 | ggtatttaca gtagtgctgt ggtcgataat gaattgaatg ctgtgaatga tggtcacctt |
| 721 | gtaagcagtc cagccatttg tagtggtagc cttagtaact tttcaaccag tgataatggg |
| 781 | tcttacagca gcaacggtag tgattttggg tcatgtgcaa gtatcacaag tggaggttca |
| 841 | tatactaaca gtgtcatcag tgacagtagt agttatactt ttccaccaag tgatgatact |
| 901 | tttttgggtg gaaacttacc ttctgacagc acctccaata gaagtgtgcc aaacaggaat |
| 961 | actactcctt gtgaaatttt ttcaagaagt acaagtacag atccttttgt ccaggatgac |
| 1021 | ttggaacatg gattagagat tatgaaattg ccagtgagca ggaacacaaa aattccacta |
| 1081 | aaacgttact cctccttagt catttttcct aggagtcctt caactacccg accgacttct |
| 1141 | ccaacaagtc tgtgtactct tctgagcaaa ggatcctatc aaacttcaca ccagtttatt |
| 1201 | atttctccta gtgaaattgc acataatgag gatggcacta gtgctaaagg atttctttca |
| 1261 | acagctgtca atggacttcg gttatctaaa acaatttgta cccccggaga agtaagagac |
| 1321 | atacggccgc ttcacaggaa gggctcgtta cagaagaaaa ttgttctttc gaataatact |
| 1381 | cccagacaga ctgtctgtga aaagtcatct gaaggatatt cttgtgtttc agtgcatttc |
| 1441 | acccaacgaa aagcagctac attagactgt gaaacaacaa atggtgattg taaaccagaa |
| 1501 | atgtcagaaa ttaagcttaa ttctgattca gagtatatta agctcatgca taggacatct |
| 1561 | gcatgtttgc catcctccca aaatgtagat tgtcaaataa atatcaatgg agaattggaa |
| 1621 | agaccacatt cacagatgaa caaaaaccat ggtattttac gaagaagtat ttcattggga |
| 1681 | ggagcttatc caaatatttc ctgtctatcc agccttaagc acaattgttc taaaggggga |
| 1741 | ccatctcagt tactcataaa gtttgcatct ggaaatgaag gtaaagtgga taatttatca |
| 1801 | agagacagca acagagattg cacaaatgaa ctgtctaatt cttgcaagac aagagatgat |
| 1861 | ttcctaggtc aagtggatgt tccactttat ccattaccga cagaaaatcc aagattggag |
| 1921 | agaccatata catttaagga ttttgttctt catccaagaa gtcacaaatc aagagttaaa |
| 1981 | ggttatctga gactaaaaat gacttattta cctaaaacca gtggctcaga agatgataat |
| 2041 | gcagaacagg ctgaggaatt agagcctggc tgggttgttt tggaccaacc agatgctgct |
| 2101 | tgccatttgc agcaacaaca agaaccttct cctctacctc cagggtggga agagaggcag |
| 2161 | gatatccttg gaaggaccta ttatgtaaac catgaatcta gaagaacaca gtggaaaaga |
| 2221 | ccaacccctc aggacaacct aacagatgct gagaatggca acattcaact gcaagcacaa |
| 2281 | cgtgcattta ccaccaggcg gcagatatcc gaggaaacag aaagtgttga caaccgagag |
| 2341 | tcttccgaga actgggaaat tataagagaa gatgaagcca ccatgtatag caaccaggcc |
| 2401 | ttcccatcac ctccaccgtc aagtaacttg gatgttccaa ctcatcttgc agaagaattg |
| 2461 | aatgccagac tcaccatttt tggaaattca gccgtgagcc agccagcatc gagctcaaat |
| 2521 | cattccagca gaagaggcag cttacaagcc tatacttttg aggaacaacc tacacttcct |
| 2581 | gtgcttttgc ctacttcatc tggattacca ccaggttggg aagaaaaaca agatgaaaga |
| 2641 | ggaagatcat attatgtaga tcacaattcc agaacgacta cttggacaaa gcccactgta |
| 2701 | caggccacag tggagaccag tcagctgacc tcaagccaga gttctgcagg ccctcaatca |
| 2761 | caagcctcca ccagtgattc aggccagcag gtgacccagc catctgaaat tgagcaagga |
| 2821 | ttccttccta aaggctggga agtccggcat gcaccaaatg ggaggccttt ctttattgac |
| 2881 | cacaacacta aaaccaccac ctgggaagat ccaagattga aaattccagc ccatctgaga |
| 2941 | ggaaagacat cacttgatac ttccaatgat ctagggcctt tacctccagg atgggaagag |
| 3001 | agaactcaca cagatggaag aatcttctac ataaatcaca atataaaaag aacacaatgg |
| 3061 | gaagatcctc ggttggagaa tgtagcaata actggaccag cagtgcccta ctccagggat |
| 3121 | tacaaaagaa agtatgagtt cttccgaaga aagttgaaga agcagaatga cattccaaac |
| 3181 | aaatttgaaa tgaaacttcg ccgagcaact gttcttgaag actcttaccg gagaattatg |
| 3241 | ggtgtcaaga gagcagactt cctgaaggct cgactgtgga ttgagtttga tggtgaaaag |
| 3301 | ggattggatt atggaggagt tgccagagaa tggttcttcc tgatctcaaa ggaaatgttt |
| 3361 | aacccttatt atgggttgtt tgaatattct gctacggaca attataccct acagataaat |
| 3421 | ccaaactctg gattgtgtaa cgaagatcac ctctcttact tcaagtttat tggtcgggta |
| 3481 | gctggaatgg cagtttatca tggcaaactg ttggatggtt ttttcatccg cccattttac |
| 3541 | aagatgatgc ttcacaaacc aataaccctt catgatatgg aatctgtgga tagtgaatat |
| 3601 | tacaattccc taagatggat tcttgaaaat gacccaacag aattggacct caggtttatc |
| 3661 | atagatgaag aactttttgg acagacacat caacatgagc tgaaaaatgg tggatcagaa |
| 3721 | atagttgtca ccaataagaa caaaaaggaa tatatttatc ttgtaataca atggcgattt |
| 3781 | gtaaaccgaa tccagaagca aatggctgct tttaaagagg gattctttga actaatacca |
| 3841 | caggatctca tcaaaatttt tgatgaaaat gaactagagc ttcttatgtg tggactggga |
| 3901 | gatgttgatg tgaatgactg gagggaacat acaaagtata aaaatggcta cagtgcaaat |
| 3961 | catcaggtta tacagtggtt ttggaaggct gttttaatga tggattcaga aaaaagaata |
| 4021 | agattacttc agtttgtcac tggcacatct cgggtgccta tgaatggatt tgctgaacta |
| 4081 | tacggttcaa atggaccaca gtcatttaca gttgaacagt ggggtactcc tgaaaagctg |
| 4141 | ccaagagctc atacctgttt taatcgcctg gacttgccac cttatgaatc atttgaagaa |
| 4201 | ttatgggata aacttcagat ggcaattgaa aacacccagg gctttgatgg agttgattag |
| 4261 | attacaaata acaatctgta gtgtttttac tgccatagtt ttataaccaa aatcttgact |
| 4321 | taaaattttc cggggaacta ctaaaatgtg gccactgagt cttcccagat cttgaagaaa |
| 4381 | atcatataaa aagcatttga agaaatagta cgacaactta tttttaatca cttttaaata |
| 4441 | atgtgttgca tttacacagt tgtttcatgc tgtctttaga gttaggtgcc tgcctaaagc |
| 4501 | caggcaccac cacacctggc tttagagttc acacaatagg atataagtcc tgtatgactt |
| 4561 | aaatagtgaa ttttgtcctt aacatttacc tcttgtatag tatctgccag gcagtttttt |
| 4621 | cttaaactac tgagatgata actgtgaaat atttgtgata cgtgtcatgt gtgaaaagtt |
| 4681 | tgatgcattt tgagatggaa aactgaaatt tggaaaaaga aatactttac tattgagtaa |
| 4741 | actacaatat atttagtgct actcgcagct atttattatt ttgtagacct gccttatgca |
| 4801 | ccttactgcc tagatttttg ggaaaaaact ttggaaagtg tgttacctat atttctagcc |
| 4861 | aactaactca cagaaaaact gtttacttct tcactttcga agtatttggc ttttgttaat |
| 4921 | atgcagtttt actaaacaga tggttcataa gacatgtgaa gcaaattcat atttgcaatg |
| 4981 | ggtaaaaagt attaaagcct ttctcttgcc tgcatatcct attgaccatt ggtatagtca |
| 5041 | tcactttttc atattttagt gtagttagaa gaattccttc ttcaaacatt aaagatccac |
| 5101 | aaagcagtat ttctaaatat gccttgaaga actaaatgaa gtgtatagca cttgccttta |
| 5161 | ctagatattc tttacacttg tacaattatg tagtaaatgt atgtttacag ggtttatcat |
| 5221 | gtttacagat taagctaatt tctgtagtcg catttttata tttttagtat cactctagta |
| 5281 | aaaaaaccaa ataatttgtt taaaataacc aaagagttgt tataatgcat aatttgtatt |
| 5341 | aaatttatta ctatttctta tgccttttaa aatactgttt actatgaaga caatgttttt |
| 5401 | aattacaaat ccagaattct gtaggcaaaa tgctacagtt catatcttcc tttaaccaaa |
| 5461 | ctgaagtaca taaagaccat gtacatgtat tcatcaaacg tttattgaat gctgcgtgcc |
| 5521 | tagcgctgtg ctatgctctg gggtaagagt tgtcagcttc agagaagctg agtcctgatc |
| 5581 | ctcaaggaac ttgcaaatgt gtctatgaat ttgtaaaaca atcaaaagta ggcgtaagca |
| 5641 | gaataaggca aaagggaaag tgttctaggt tccagcacac ctgcaaagat aaagtgtgcc |
| 5701 | aagactgtat ttatatttca tacttatatt gtttcatcct tatattggaa tgattatata |
| 5761 | gaaaatgctc ttaaaaagat taaacctatt tctcagtatg gtatcttggt gatttaggaa |
| 5821 | taattgtaaa tatatgttac gaatcttctt aaatatatat atatacacac cctgtgagaa |
| 5881 | ctgtaaaaag tacctctggt tcttggttta agtttgttgg ggtataacat gatgagtact |
| 5941 | cattagcacc tgatagaaat ctgaaatgtg acagtagcaa aaccactttc tactttccaa |
| 6001 | acaccacagc atcagcatgg tttaggggaa gcaattcaca gattaatgta ccctgcgttt |
| 6061 | tgtcttcacc attgtcaacc agcagtcaag gatgagcacc aggtatagtc ctggtttgat |
| 6121 | atctcaccag ccatgtgaac tgaagcagct tacttaacct ctctggattc atatttcttc |
| 6181 | atctgtcaaa tgagagtaat catgcccacc ctcattgtca tgccagattg tcagaagttc |
| 6241 | cagacagatg agaaagcaaa aagtactttg taaagtgtta tacaatcaga aggtgctttg |
| 6301 | tgctgaccgt cagattgtgt caagtcaggt gtgcaaattg accacactcc caaagaatca |
| 6361 | tttaaaagta aagtgacttc taagagacaa ggaagtagga acatttctga tattaaatat |
| 6421 | ataaataata tggatacaca tgtgtatatc tgtatatgga tcttaccatc ataattcatt |
| 6481 | ttcttcatat cagcaaagta atagtaatgt gggccaatgc attttggaaa tgtcctcatt |
| 6541 | atgtagaatg gaatgtgaaa attatttttg ttaaaacagg attttgccac aattatttaa |
| 6601 | atattatgtt tgtgaactat ctaagcatga gaaaatacaa agctttcttt gtatctggct |
| 6661 | gatactcatt tggatgattc tgtgactcat agaacatgat gttaaatgag accagactca |
| 6721 | actgccacca gtccccagct gcagaccttc atccccttca tctcccacta ggggctcgtg |
| 6781 | gtctggaaga aacatctatc aagcaacaca gccccttatc ccagatagaa aagtgtctca |
| 6841 | aatgcattct actgcgggac agtcagtagg atcattttca taaagcaagg acatcatatg |
| 6901 | tttctagaaa ttacaagcat ataactttag cctacaatct cttattaaaa atttttaaca |
| 6961 | aaattgtatt acaaatgcat ttcatcagaa ctcaaattta aatggtgttt gttttgggtt |
| 7021 | tttatttata atgctgaagt tattccatat aagtatcaag ttaaacacaa ttcattttga |
| 7081 | ttataaacta tttgactttt taaaatcttc tgacacagta aatatatata tcaagattga |
| 7141 | tgtatcaaaa tttattgcac actttaaagt gtaaaatcat tttttaaaat cttgaatcca |
| 7201 | caaataaagt tctattctga ttttaaaaaa caaaacaaaa |

By “WW domain containing E3 ubiquitin protein ligase 1 (WWP1) polypeptide” is meant a protein having about 85% amino acid sequence identity to NCBI Reference Sequence: NP_008944.1 and having E3 ligase activity. WWP1 is frequently overexpressed in cancers, such as, for example, prostate cancer, breast cancer, gastric carcinoma, and liver cancer. An exemplary WWP1 amino acid sequence is provided herein below (SEQ ID NO: 5):

| | |
|---|---|
| 1 | matasprsdt snnhsgrlql qvtvssaklk rkknwfgtai ytevvvdgei tktaksssss |
| 61 | npkwdeqltv nvtpqttlef qvwshrtlka dallgkatid lkqallihnr klervkeqlk |
| 121 | lslenkngia qtgeltvvld glvieqenit ncsssptiei qengdalhen gepsarttar |
| 181 | lavegtngid nhvptstlvq nsccsyvvng dntpsspsqv aarpkntpap kplasepadd |
| 241 | tvngesssfa ptdnasvtgt pvvseenals pnctsttved ppvqeiltss ennecipsts |
| 301 | aelesearsi lepdtsnsrs ssafeaaksr qpdgcmdpvr qqsgnantet lpsgweqrkd |
| 361 | phgrtyyvdh ntrtttwerp qplppgwerr vddrrrvyyv dhntrtttwq rptmesvrnf |
| 421 | eqwqsqrnql qgamqqfnqr ylysasmlaa endpygplpp gwekrvdstd rvyfvnhntk |
| 481 | ttqwedprtq glqneeplpe gweirytreg vryfvdhntr tttfkdprng kssvtkggpq |
| 541 | iayergfrwk lahfrylcqs nalpshvkin vsrqtlfeds fqqimalkpy dlrrrlyvif |
| 601 | rgeegldygg larewfflls hevlnpmycl feyagknnyc lqinpastin pdhlsyfcfi |
| 661 | grfiamalfh gkfidtgfsl pfykrmlskk ltikdlesid tefynsliwi rdnnieecgl |
| 721 | emyfsvdmei lgkvtshdlk lggsnilvte enkdeyiglm tewrfsrgvq eqtkafldgf |
| 781 | nevvplqwlq yfdekelevm lcgmqevdla dwqrntvyrh ytrnskqiiw fwqfvketdn |
| 841 | evrmrllqfv tgtcrlplgg faelmgsngp qkfciekvgk dtwlprshtc fnrldlppyk |
| 901 | syeqlkekll faieetegfg qe |

By “WWP1 polynucleotide” is meant a nucleic acid molecule encoding a WWP1 polypeptide. An exemplary WWP1 polynucleotide sequence is provided at NCBI Reference Sequence: NM_007013, and reproduced herein below (SEQ ID NO: 6):

| | |
|---|---|
| 1 | ggctgctggc ggcctgggct gccggggccg acgcctgggt ggctgctgcc gccgcgcctg |
| 61 | ctgcgagatg gcgatcttgg gcgcggaagg gtgagggcgc ccgccgcagg aggaggtgcc |
| 121 | gctgccgtgg ccgcccggct gccgggagcc gacagcttcg cgccggggtt gtctcctcac |
| 181 | agactatgag ctccttgaaa gagggaatcg tgtcttactc atctttgtat ccccagtgtc |
| 241 | tagcagttcc tgatacatag ttttagctga attttgggac atggccactg cttcaccaag |
| 301 | gtctgatact agtaataacc acagtggaag gttgcagtta caggtaactg tttctagtgc |
| 361 | caaacttaaa agaaaaaaga actggttcgg aacagcaata tatacagaag tagttgtaga |
| 421 | tggagaaatt acgaaaacag caaaatccag tagttcttct aatccaaaat gggatgaaca |
| 481 | gctaactgta aatgttacgc cacagactac attggaattt caagtttgga gccatcgcac |
| 541 | tttaaaagca gatgctttat taggaaaagc aacgatagat ttgaaacaag ctctgttgat |
| 601 | acacaataga aaattggaaa gagtgaaaga acaattaaaa ctttccttgg aaaacaagaa |
| 661 | tggcatagca caaactggtg aattgacagt tgtgcttgat ggattggtga ttgagcaaga |
| 721 | aaatataaca aactgcagct catctccaac catagaaata caggaaaatg gtgatgcctt |
| 781 | acatgaaaat ggagagcctt cagcaaggac aactgccagg ttggctgttg aaggcacgaa |
| 841 | tggaatagat aatcatgtac ctacaagcac tctagtccaa aactcatgct gctcgtatgt |
| 901 | agttaatgga gacaacacac cttcatctcc gtctcaggtt gctgccagac ccaaaaatac |
| 961 | accagctcca aaaccactcg catctgagcc tgccgatgac actgttaatg gagaatcatc |
| 1021 | ctcatttgca ccaactgata atgcgtctgt cacgggtact ccagtagtgt ctgaagaaaa |
| 1081 | tgccttgtct ccaaattgca ctagtactac tgttgaagat cctccagttc aagaaatact |
| 1141 | gacttcctca gaaaacaatg aatgtattcc ttctaccagt gcagaattgg aatctgaagc |
| 1201 | tagaagtata ttagagcctg acacctctaa ttctagaagt agttctgctt ttgaagcagc |
| 1261 | caaatcaaga cagccagatg ggtgtatgga tcctgtacgg cagcagtctg ggaatgccaa |
| 1321 | cacagaaacc ttgccatcag ggtgggaaca aagaaaagat cctcatggta gaacctatta |
| 1381 | tgtggatcat aatactcgaa ctaccacatg ggagagacca caacctttac ctccaggttg |
| 1441 | ggaaagaaga gttgatgatc gtagaagagt ttattatgtg gatcataaca ccagaacaac |
| 1501 | aacgtggcag cggcctacca tggaatctgt ccgaaatttt gaacagtggc aatctcagcg |
| 1561 | gaaccaattg cagggagcta tgcaacagtt taaccaacga tacctctatt cggcttcaat |
| 1621 | gttagctgca gaaaatgacc cttatggacc tttgccacca ggctgggaaa aaagagtgga |
| 1681 | ttcaacagac agggtttact ttgtgaatca taacacaaaa acaacccagt gggaagatcc |
| 1741 | aagaactcaa ggcttacaga atgaagaacc cctgccagaa ggctgggaaa ttagatatac |
| 1801 | tcgtgaaggt gtaaggtact ttgttgatca taacacaaga acaacaacat tcaaagatcc |
| 1861 | tcgcaatggg aagtcatctg taactaaagg tggtccacaa attgcttatg aacgcggctt |
| 1921 | taggtggaag cttgctcact tccgttattt gtgccagtct aatgcactac ctagtcatgt |
| 1981 | aaagatcaat gtgtcccggc agacattgtt tgaagattcc ttccaacaga ttatggcatt |
| 2041 | aaaaccctat gacttgagga ggcgcttata tgtaatattt agaggagaag aaggacttga |
| 2101 | ttatggtggc ctagcgagag aatggttttt cttgctttca catgaagttt tgaacccaat |
| 2161 | gtattgctta tttgagtatg cgggcaagaa caactattgt ctgcagataa atccagcatc |
| 2221 | aaccattaat ccagaccatc tttcatactt ctgtttcatt ggtcgtttta ttgccatggc |
| 2281 | actatttcat ggaaagttta tcgatactgg tttctcttta ccattctaca agcgtatgtt |
| 2341 | aagtaaaaaa cttactatta aggatttgga atctattgat actgaatttt ataactccct |
| 2401 | tatctggata agagataaca acattgaaga atgtggctta gaaatgtact tttctgttga |
| 2461 | catggagatt ttgggaaaag ttacttcaca tgacctgaag ttgggaggtt ccaatattct |
| 2521 | ggtgactgag gagaacaaag atgaatatat tggtttaatg acagaatggc gtttttctcg |
| 2581 | aggagtacaa gaacagacca aagctttcct tgatggtttt aatgaagttg ttcctcttca |
| 2641 | gtggctacag tacttcgatg aaaaagaatt agaggttatg ttgtgtggca tgcaggaggt |
| 2701 | tgacttggca gattggcaga gaaatactgt ttatcgacat tatacaagaa acagcaagca |
| 2761 | aatcatttgg ttttggcagt ttgtgaaaga gacagacaat gaagtaagaa tgcgactatt |
| 2821 | gcagttcgtc actggaacct gccgtttacc tctaggagga tttgctgagc tcatgggaag |
| 2881 | taatgggcct caaaagtttt gcattgaaaa agttggcaaa gacacttggt taccaagaag |
| 2941 | ccatacatgt tttaatcgct tggatctacc accatataag agttatgaac aactaaagga |
| 3001 | aaaacttctt tttgcaatag aagagacaga gggatttgga caagaatgaa tgtggcttct |
| 3061 | tattttggag gagctcttgc atttaaatac cccagccaag aaaaattgca cagatagtgt |
| 3121 | atataagctg ttcattctgt acagtgaatt ttccgaacct ctcaaagtat gttttccgtt |
| 3181 | cttccacaga aatatgcaaa acagttcatc cttttctact ttatttattg ttcccttgaa |
| 3241 | atgactgacc aggaaaaaga tcatccttaa attttgaagc aagtgagaga ctttattaaa |
| 3301 | aatacatata tatctatata aacatatatg atagtggctc tagttttata gagctccaag |
| 3361 | tgtattaaac atgacagcca ttcattcata aagatctgga tttgctttac cttgttaata |
| 3421 | ttatctaggg gaaaaagtgc aaattgctcc atgttcttct ctcccttatg taacatctcc |
| 3481 | tgagggtgtt tagttgcatg gctgttcaga aaggtattaa gggcttaggc caaatcttac |
| 3541 | tttgagtatg ttaaaaaaaa aaaaatgctg ctggcttttc tgaagacagg tgcttgaact |
| 3601 | tgtcagtttg ttttaaataa atacaatagt tgaaaatttt tctctgttac atcagtaata |
| 3661 | ttgttaaagt aatggataga accataactt acacatgaaa gtcatatact agatccaata |
| 3721 | ctatttagtt tattatcgaa attggaagga ttcattgagc agcatagaag tttgtttaca |
| 3781 | tgttactttg agatgctagg tatttgtgga attaaaaaga atcaggctct tttgtacttt |
| 3841 | gtttttaaat ctgtgatgct tttcaaattt aattcataat aaattgatgc aatttcatac |
| 3901 | ttaggaacat acaaaaggta atgtaaactc tgccactttt ttgtgttcaa aattttggtt |
| 3961 | tttatgaagc cagatggatt gaagagttac ataagcattt gaatgctcta atataaggct |
| 4021 | aatgattttc tgttagtgtt tgaatatctt cattcctctc aaattcataa cagttctatt |
| 4081 | taactgaatt aaataaccat atgaaaaaaa aaaaaaaaaa |

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include any cancer, including but not limited to breast cancer, prostate cancer, and colon cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein. The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Cancers of the invention are those characterized by a reduction in, or an alteration in, or the loss of markers Pten and p53.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof Δny compositions or methods provided herein can be combined with one or more of any of the other compositions and methods provided herein.

### BRIEF DESCRIPTION OF THE DRAWINGS

FIG. 1A-1E shows WWP1/NEDD4-1 is required for PTEN K27-link poly-ubiquitination, which is not a signal for protein degradation.

FIG. 1A is a Western blot analysis of PTEN K27-linked ubiquitination in steady state conditions. 293T lysates were transfected with PTEN along with His-ubiquitin (His-Ub), and the indicated His-Ub KR mutants in the absence of proteasome inhibitor MG132; lysates were collected for Ni-NTA pull down, followed by western blot analysis.

FIG. 1B shows results of a co-immunoprecipitation analysis of the interaction between Myc-PTEN and individual Flag-tagged NEDD4-1 family ubiquitin E3 ligase. Immunoprecipitation (IP) of Flag-NEDD4-1 family ligases with Flag antibody, and then probed with PTEN antibodies to detect PTEN/individual NEDD4-1 family ligase interactions.

FIG. 1C is a Western blot analysis of PTEN K27-linked poly-ubiquitination in DU145 cells expressing indicated NEDD4-1 family ubiquitin ligases as in FIG. 1A.

FIG. 1D is a Western blot. DU145 cells transfected with indicated constructs were treated with 100 μg/ml cyclohexmide for various time points and endogenous PTEN was analysed by western blot and ImageJ software.

FIG. 1E is a Western blot. PC3 cells transfected with indicated constructs were treated with 100 μg/ml cyclohexmide for various time points and exogenous PTEN was analysed by western blot and ImageJ software.

FIG. 2A-2K show the identification characterization of a WWP1/NEDD4-1 E3 for PTEN K27-linked poly-ubiquitination.

FIG. 2A is a Western blot. Lysates from DU145 cells transfected with HA-PTEN were immunoprecipitated with an anti-PTEN antibody followed by mass-spectrometric peptide sequencing. WWP1 and NEDD4-1 were identified.

FIG. 2B is a Western blot showing that endogenous WWP1, NEDD4-1, and PTEN form complex in vivo. DU145 cells were immunoprecipitated with anti-PTEN antibody and then analysed by western blot.

FIG. 2C is a Western blot showing that WWP1 binds NEDD4-1 in vitro. Recombinant Flag-WWP1 were incubated with immunopurified recombinant NEDD4-1, and then analysed by western blot.

FIG. 2D is a Western blot showing that effects of indicated ubiquitin KR mutants on WWP1/NEDD4-1-mediated PTEN poly-ubiquitination, 293T cells were transfected with indicated constructs, and PTEN ubiquitination was analysed. The ubiquitinated proteins were pulled down under denaturing conditions by Ni-NTA agarose, and analysed by western blot.

FIG. 2E is a Western blot showing an analysis of PTEN K27-linked poly-ubiquitination in PC3 cells expressing the indicated NEDD4-1 family ubiquitin ligases as in FIG. 1D.

FIGS. 2F and 2G show a Western blot analysis of in vitro ubiquitination of PTEN by WWP1/NEDD4-1 E3 complex. Flag-PTEN purified from 293 cells was subject to in vitro ubiquitination reaction in the presence of E1, E2, E3 complex, and ubiquitin or ubiquitin various mutants, and then examined by western blot with anti-PTEN antibody.

FIG. 2H provides a flow chart of Tandem mass analysis (upper). Tandem mass spectrum of a peptide derived from ubiquitinated PTEN showing ubiquitin conjugation at the K27 residue of ubiquitin (bottom).

FIG. 2I is a graph showing the ratio of indicated ubiquitin linkages detected by MS analysis of ubiquitinated PTEN purified from WWP1/NEDD4-1 overexpression cells to that from control cells. The abundance of each ubiquitin linkages was calculated as described in Methods.

FIG. 2J is a tandem mass spectra of peptides derived from ubiquitinated PTEN showing ubiquitin conjugation at amino acids 342 (top) and 344 (bottom). Ions labeled with “0” indicate a neutral loss of H₂O.

FIGS. 3A-3G show the oncogenic role of K27-linked poly-ubiquitination.

FIG. 3A provides a Western blot analysis of AKT activation and ubiquitin expression levels in various ubiquitin replacement cell lines treated with doxycycline for 2 Days.

FIG. 3B provides a Western blot analysis of PTEN poly-ubiquitination in various ubiquitin cell lines treated with doxycycline for 2 Days.

FIG. 3C shows the conservation of K342/K344 patch between different species.

FIG. 3D shows the effects of PTEN K342/K344Rr mutant on proliferation of PC3 cells. In vitro proliferation rate of PC3 cells as in FIG. 5N, P.

FIG. 3E is a photograph showing that PC3 cells were assayed, as also described in FIG. 5N, P, for their colony forming ability in soft agar. The colony number are quantified and presented as mean±SD (***P<0.0005, **P<0.005, n=3).

FIG. 3F shows tumour growth of PC3 cells expressing indicated constructs in a xenograft model.

FIG. 3G provides two micrographs showing that tumours derived from each cell lines were analysed by PTEN staining. The boxed areas are enlarged two fold to show on the right. Bar, 50 μm.

FIG. 4A-4G shows the role of WWP1/NEDD4-1 E3 in K27-linked PTEN poly-ubiquitination.

FIGS. 4A and 4B provide a Western blot analyses of WWP1-mediated or NEDD4-1-mediated PTEN K27-linked poly-ubiquitination in DU145 cells expressing indicated siRNAs as in FIG. 1D. RT-qPCR analysis of WWP1, TRIM27, ITCH, and RNF168 in DU145 expressing indicated siRNAs. The knockdown efficiency of individual siRNAs was determined by RT-qPCR, and is presented as a percentage of decrease in mRNA level. (FIG. 1A; bottom).

FIG. 4C provides a Western blot analysis of PTEN K27-linked poly-ubiquitination in 293 cells expressing indicated WT or catalytic inactive WWP1 and/or NEDD4-1 ligases.

FIG. 4D is a schematic representation of the indicated region of PTEN used in this study (upper). Western blot analysis of reciprocal co-immunoprecipitation of PTEN with WWP1 from 293 cells expressing indicated plasmids (bottom).

FIG. 4E shows mapping the binding domain of WWP1 with PTEN. Lysates of 293 cells transfected with indicated constructs were analysed by immunoprecipitation and/or western blot with indicated antibodies.

FIG. 4F shows a co-immunoprecipitation analysis of the interaction between Flag-WWP1 and PTEN individual mutants. Immunoprecipitation (IP) of Flag-WWP1 with Flag-tag antibody, and then probed with PTEN antibodies to detect individual PTEN mutants/WWP1 interactions.

FIG. 4G shows effects of indicated PTEN mutants on WWP1/NEDD4-1-mediated PTEN poly-ubiquitination. 293 cells were transfected with indicated constructs, and PTEN ubiquitination was analysed as in FIG. 1D.

FIG. 5A-5P shows K27-linked PTEN poly-ubiquitination suppresses PTEN dimerization, membrane recruitment and function.

FIG. 5A provides a schematic description of the in vitro binding analysis of Flag-tagged un-modified/ubiquitinated PTEN with GST-PTEN from bacteria (upper). In vitro pull down assay with indicated Flag-tagged unmodified/ubiquitinated PTEN and GST-PTEN. Flagged untagged PTEN or ubiquitinated PTEN was purified from 293 cells transfected with Flag-PTEN, WWP1/NEDD4-1, along with individual His-ubiquitin variants using M2 beads; whereas GST-PTEN is purified from bacteria (bottom).

FIG. 5B provides a Western blot analysis of endogenous PTEN dimerization/oligomerization in DU145 cells by non-reducing gel. DU145 cells transfected with indicated constructs were immunoprecipitated with anti-rabbit PTEN antibody; native elution and western blot shows PTEN dimer and/or oligomer by using a mouse anti-PTEN antibody.

FIG. 5C shows membrane and soluble fractions isolated from DU145 cells transfected with indicated constructs were analysed by western blot. EGFR serves as membrane marker, and Actin as the internal control.

FIG. 5D provides a Western blot analysis of AKT activation in DU145 cells. Total lysates were resolved by SDS-PAGE and then probed with indicated antibodies.

FIG. 5E shows PTEN subcellular localization in wild type (WT) or K27R ubiquitin replacement cells treated with or without doxycycline. Representative confocal images are shown. Arrow indicates the PTEN plasma membrane localization. Bar, 20 μm.

FIG. 5F shows a PTEN dimerization analysis in Wwp1^{+/+} and Wwp1^{-/-} MEFs, as in FIG. 3B.

FIG. 5G shows an analysis of AKT activation in Wwp1^{+/+} and Wwp1^{-/-} MEFs.

FIG. 5H shows membrane and soluble fractions isolated from Wwp1^{+/+} and Wwp1^{-/-} MEFs were analysed by western blot. EGFR serves as a membrane marker, and Actin as the internal control.

FIG. 5I shows a PTEN dimerization analysis in Nedd4^{+/+} and Nedd4^{-/-} MEFs, as in FIG. 5B.

FIG. 5J shows that membrane and soluble fractions isolated from Wwp1^{+/+} and Wwp1^{-/-} MEFs were analysed by western blot. EGFR serves as a membrane marker, and Actin as the internal control, as in FIG. 5H.

FIG. 5K shows the effects of PTEN K342/K344R mutant on PTEN dimerization in PC3 cells. PC3 cells transfected with indicated constructs were serum starved for 6 hours and then treated with 100 ng/ml insulin for 10 minutes. Total lysates were immunoprecipitated with a rabbit anti-PTEN antibody; native elution and western blot shows monomer and dimer of the protein, as indicated by arrows, by using a mouse anti-PTEN antibody.

FIG. 5L shows the effects of PTEN K342/K344R mutant on PTEN dimer/oligomer formation in 293 cells. Lysates from HEK293 cells transfected with Myc-PTEN or Myc-PTEN K342/K344R mutant were separated by gel filtration. Fractions were resolved by SDS-PAGE and probed with anti-Myc antibody.

FIG. 5M shows the membrane localization of PTEN K342/K344R mutant in PC3. PC3 cells transfected with indicated constructs were serum-starved for 6 hours and treated with 100 ng/ml insulin for 10 minutes. Membrane and soluble fractions isolated from PC3 cells were analysed by western blot. EGFR serves as the marker for the membrane fraction, and Actin as the internal control for the soluble fraction.

FIG. 5N shows the subcellular localization of PTEN K342/K344R mutant in PC3. PC3 cells were serum-starved for 6 hours and treated with 100 ng/ml insulin for 10 minutes. Confocal images of PC3 cells stably expressing indicated PTEN WT or K342/K344R mutant stained with DAPI and indicated antibodies. Arrow indicates the PTEN plasma membrane localization. Bar, 20 μm. The percentage of cells displaying PTEN plasma membrane localization was quantified (bottom panel). Data are mean±SD; n=3, fifty cells per group per experiment.

FIG. 5O provides a panel of micrographs showing the effects of indicated PTEN KR mutants on AKT activation in PC3 cells. PC3 cells transfected with indicated PTEN KR mutants were serum-starved for 6 hours and treated with 100 ng/ml insulin for 10 minutes. Total lysates were harvested and then probed with indicated antibodies.

FIG. 5P is a graph showing the effects of PTEN K342/K344R mutant on tumour growth of PC3 cells as in (N) in a xenograft mouse model. Error bar represents SEM (n=5 mice/group).

FIG. 6A-6O shows MYC trans-activates WWP1/NEDD4-1 gene expression towards PTEN suppression and the human relevance of WWP1/NEDD4-1/PTEN axis in prostate cancer progression.

FIG. 6A is a schematic description of MYC responsive element on Wwp1 and Nedd4-1 promoter (upper). Chromatin level of MYC at promoters of human Wwp1 and Nedd4-1 were performed in DU145 cells. Fold enrichment of MYC was determined by quantitative chromatin immunoprecipitation (qChIP) assays. TSS, transcription start site. Data are shown as mean±SD (***P<0.0005, **P<0.005, n=3).

FIG. 6B is an RT-qPCR analysis of WWP1 and NEDD4-1 in DU145 cells expressing the indicated constructs. The mRNA level of WWP1 and NEDD4-1 was determined by RT-qPCR, and is presented as a fold increase, as compared to the vector control.

FIGS. 6C and 6D show an analysis of WWP1/NEDD4-1/PTEN expression and AKT activation in DU145 cells expressing different amounts of HA-MYC or indicated siRNA SMARTpool. Total lysates were resolved by SDS-PAGE and then probed with indicated antibodies.

FIG. 6E shows an analysis of PTEN K27-linked poly-ubiquitination in DU145 cells stably expressing MYC and/or WWP1/NEDD4-1 shRNAs.

FIG. 6F shows a co-immunoprecipitation analysis of the interaction between Myc-PTEN and GFP-PTEN in DU145 cells transfected with indicated plasmids.

FIG. 6G shows membrane localization of endogenous PTEN in DU145 cells expressing different doses of MYC. Membrane and soluble fractions isolated from cells were analysed by western blot. EGFR serves as membrane marker.

FIG. 6H shows an analysis of WWP1/NEDD4-1/PTEN expression and AKT activation in DU145 cells stably expressing MYC and/or WWP1/NEDD4-1 shRNAs.

FIG. 61 shows an analysis of WWP1/NEDD4-1/PTEN and AKT activation in DU145 cells expressing indicated siRNAs.

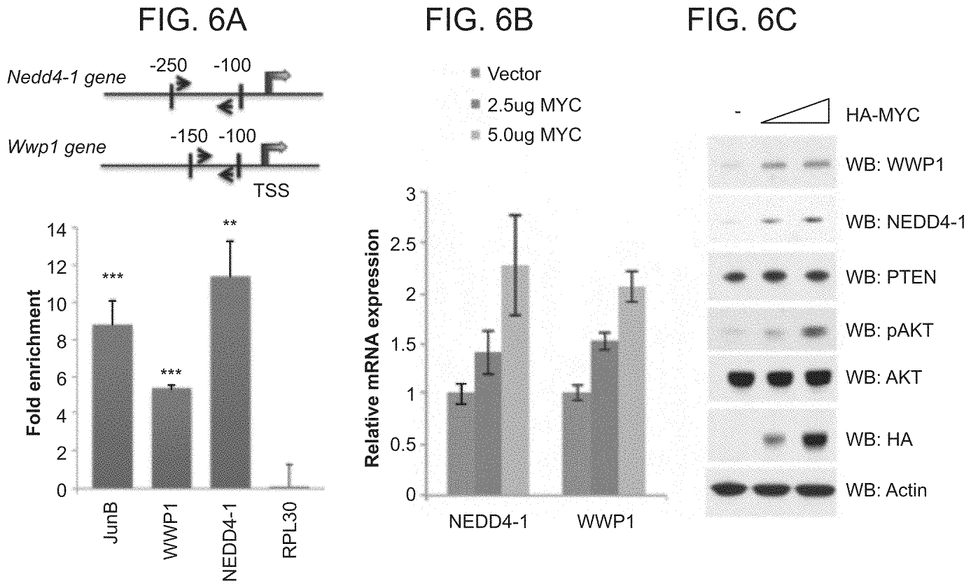

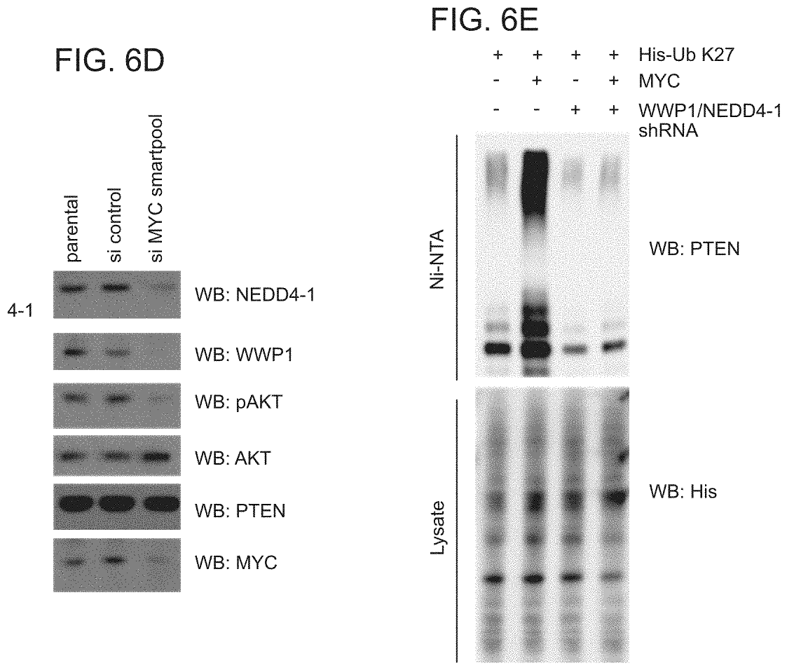

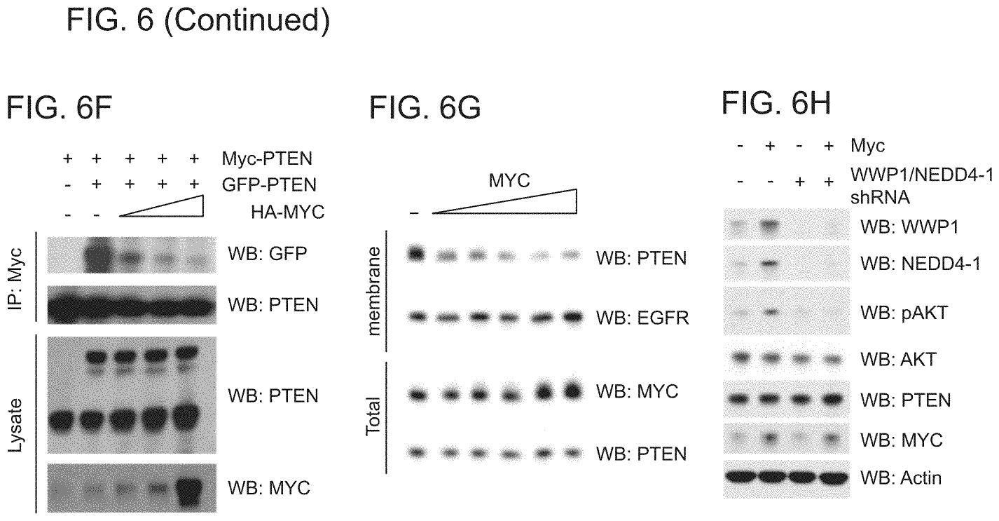

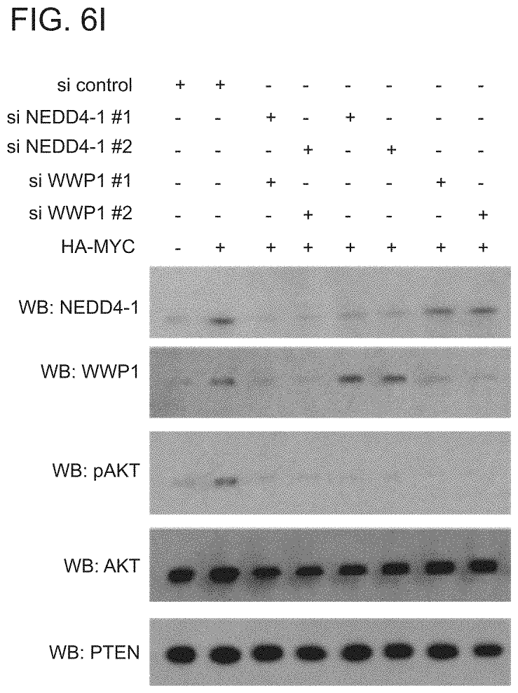

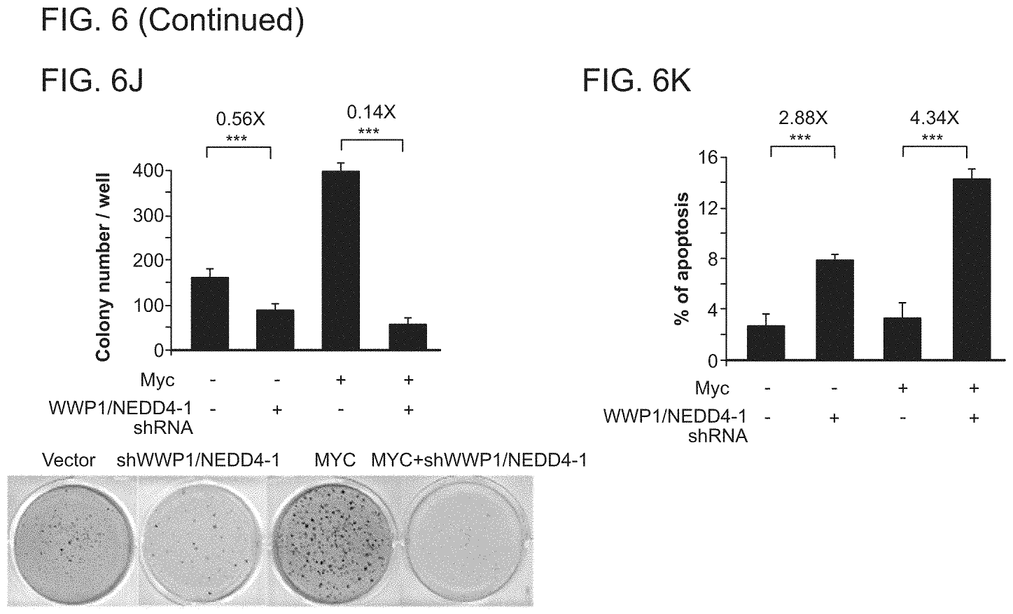

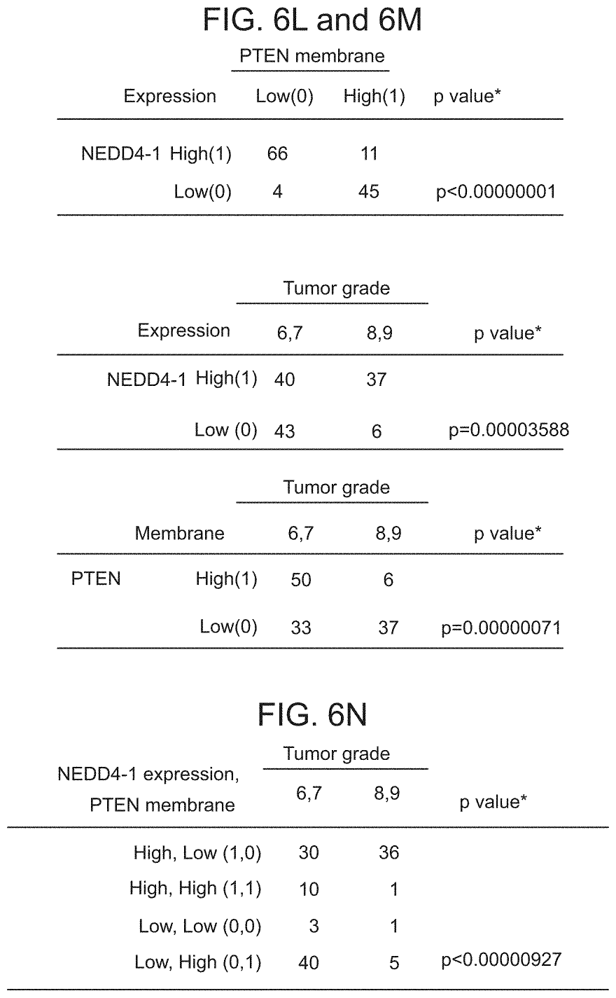

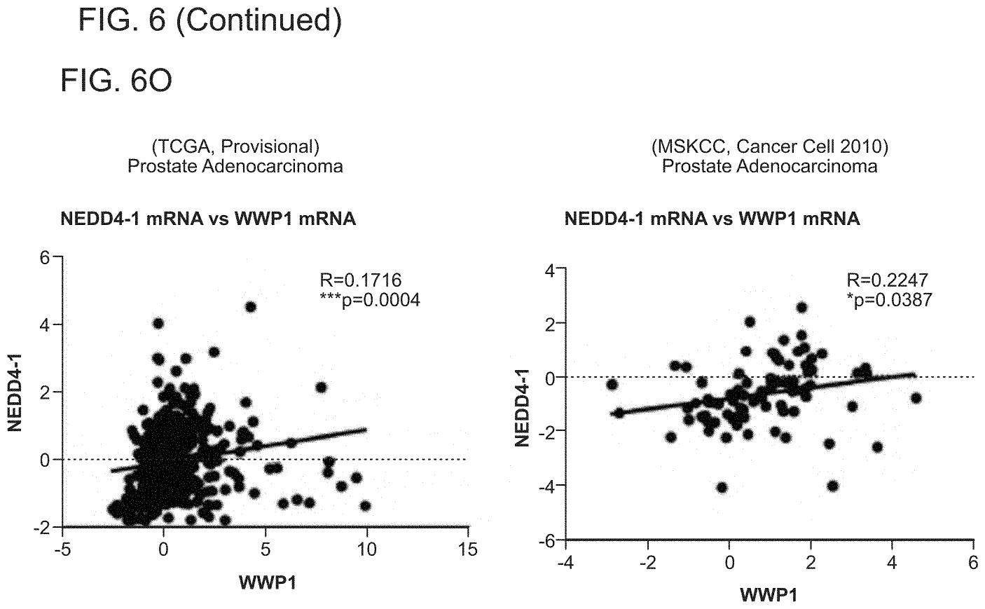

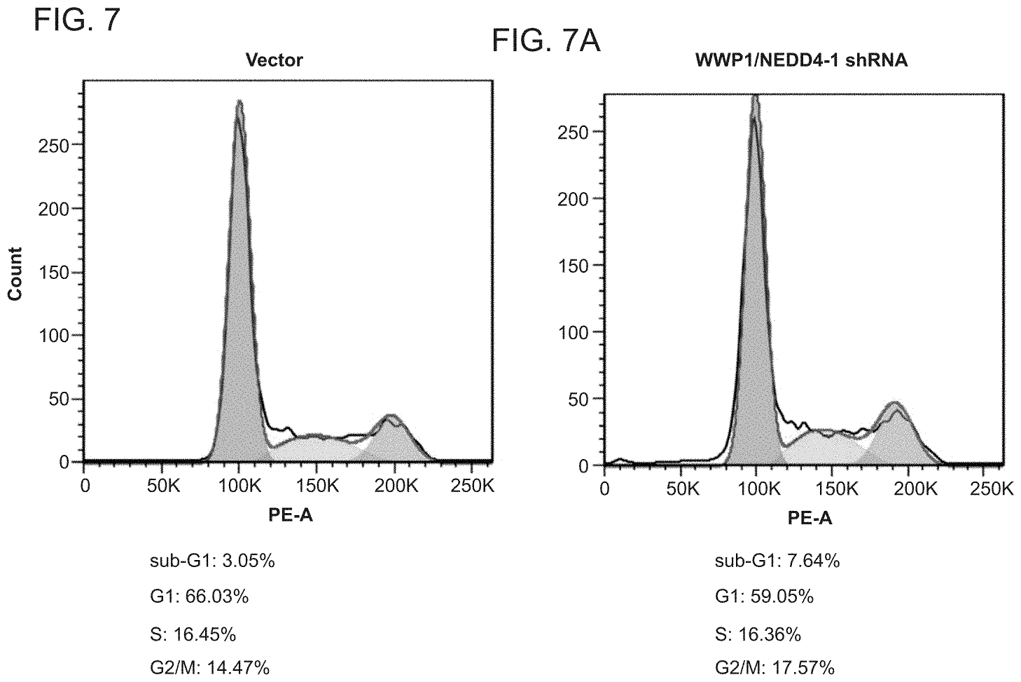

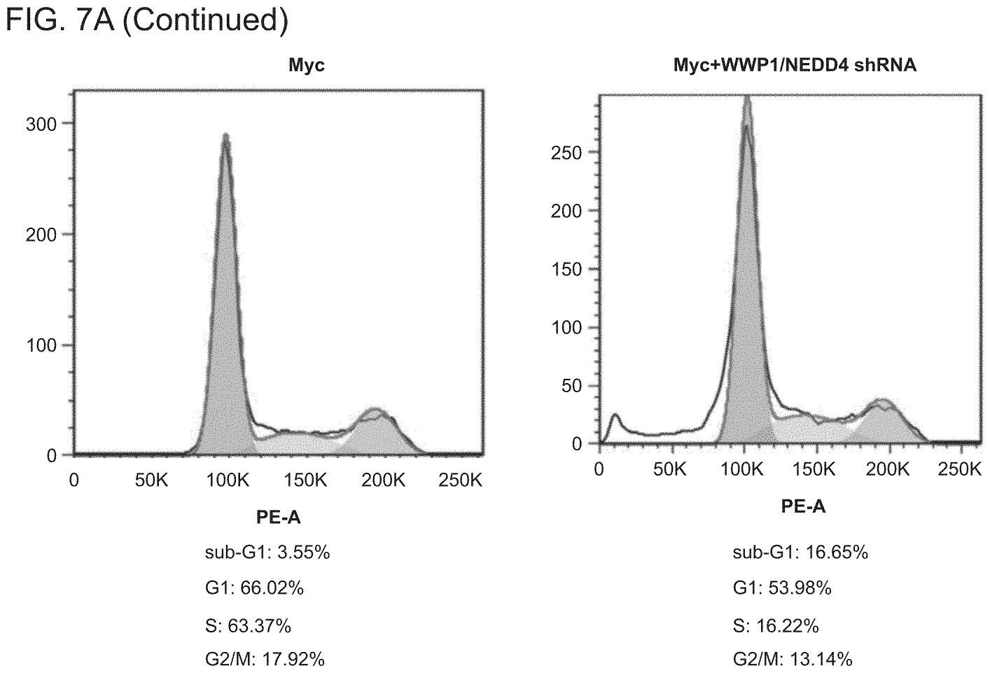

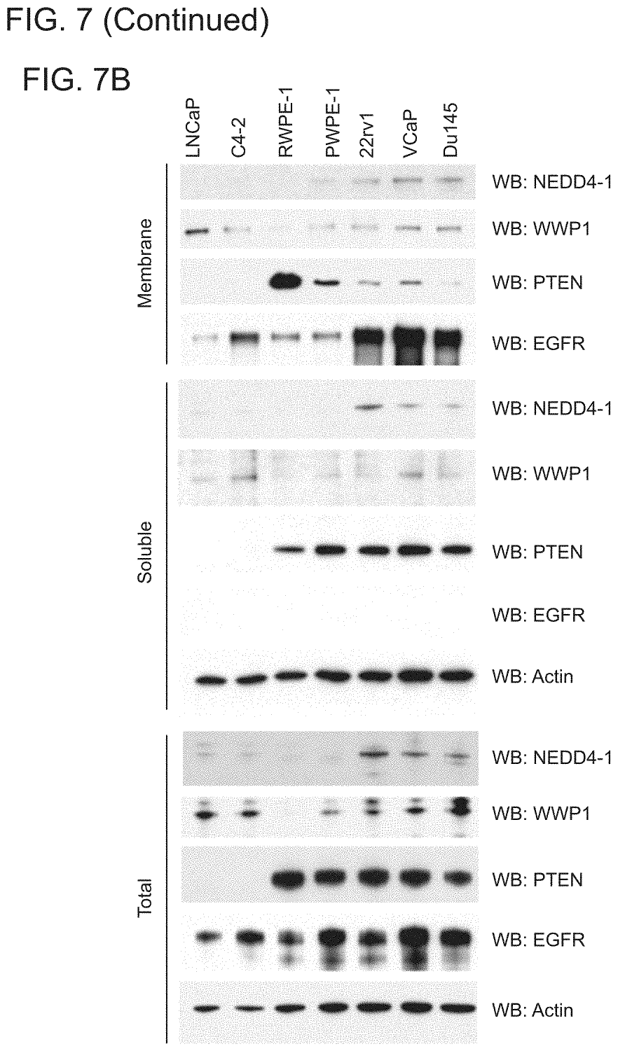

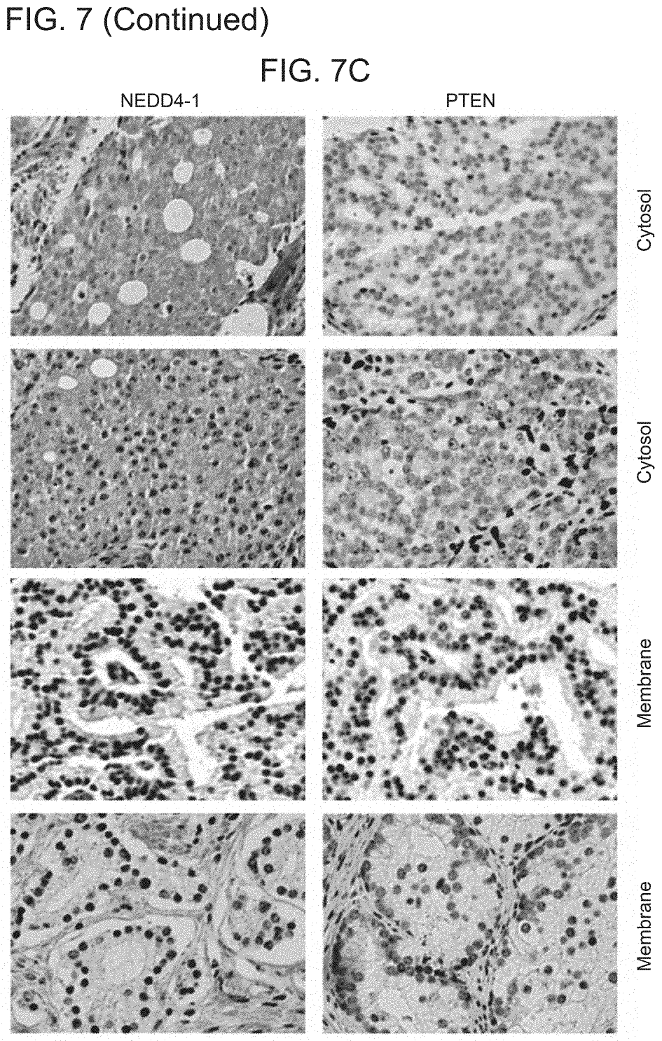

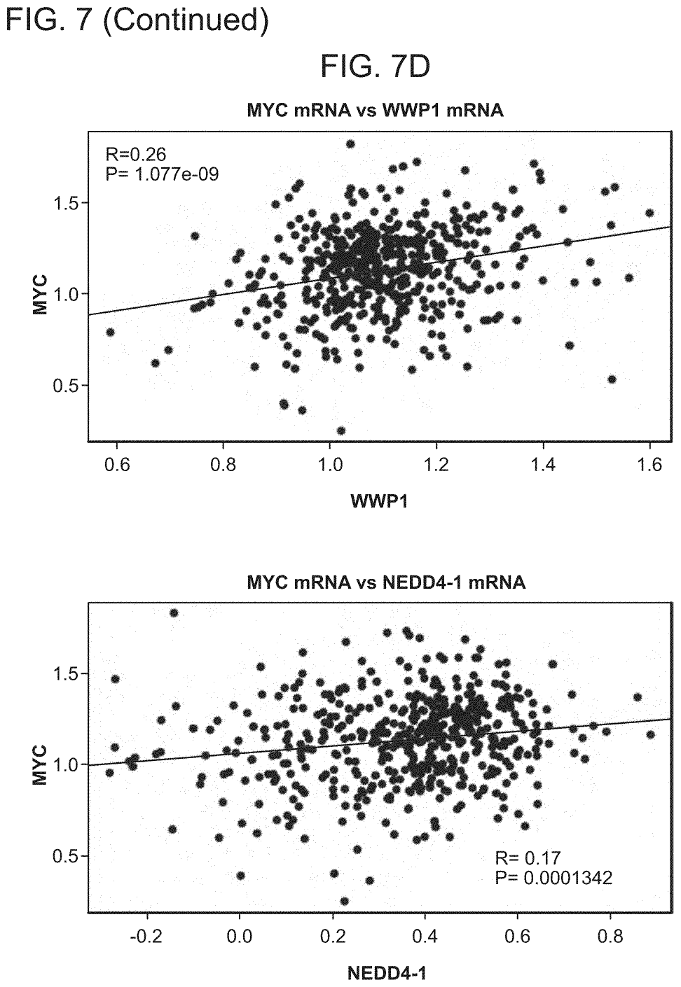

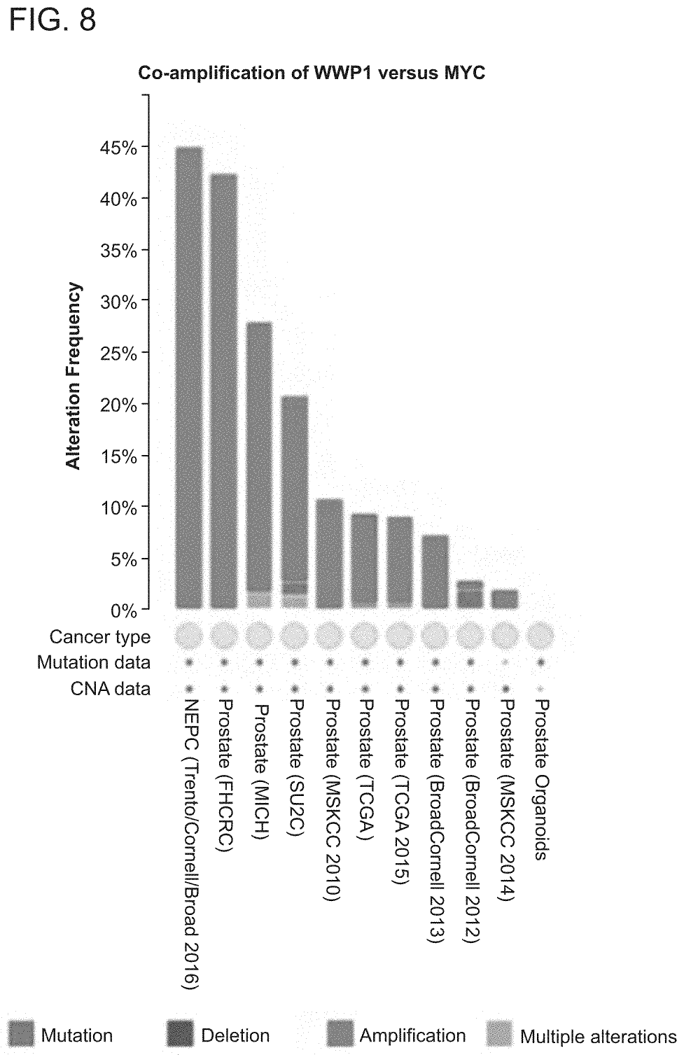

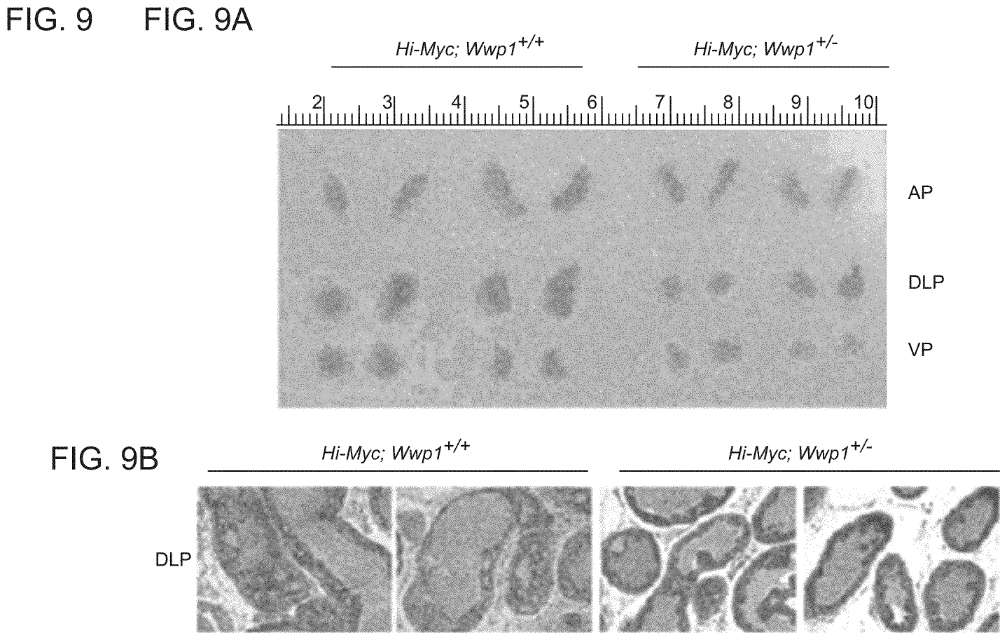

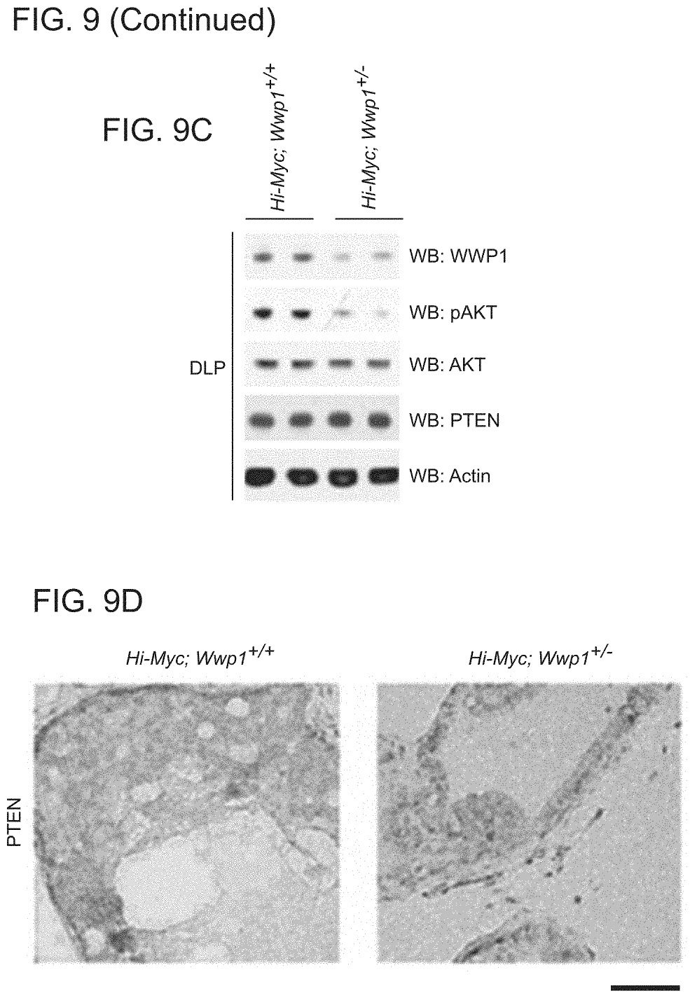

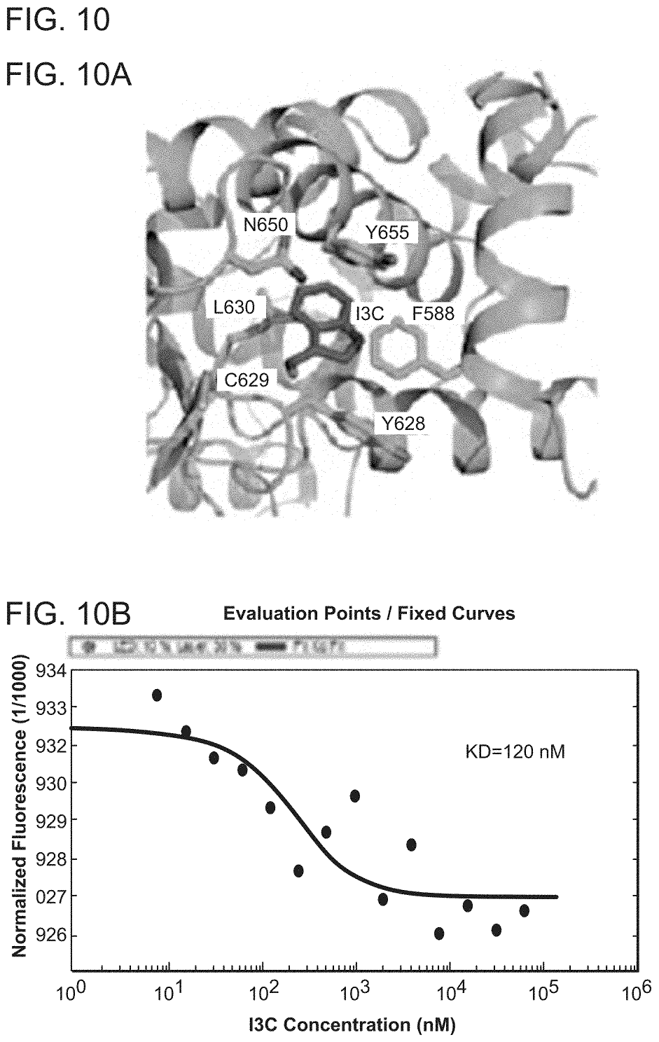

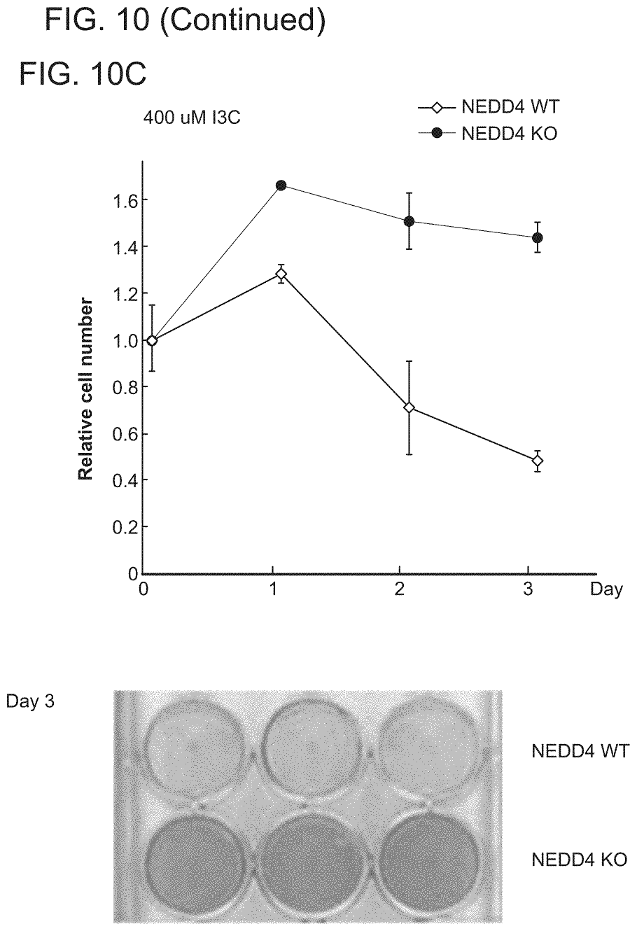

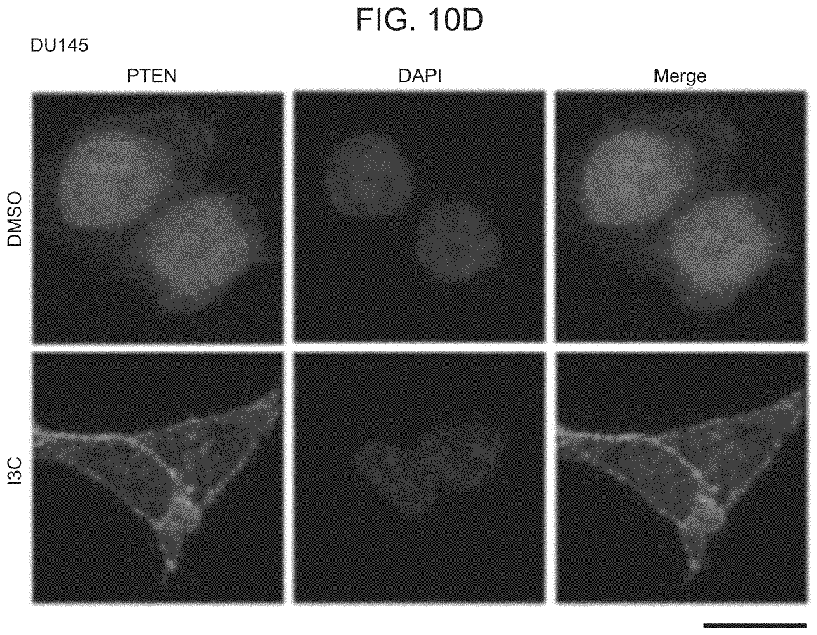

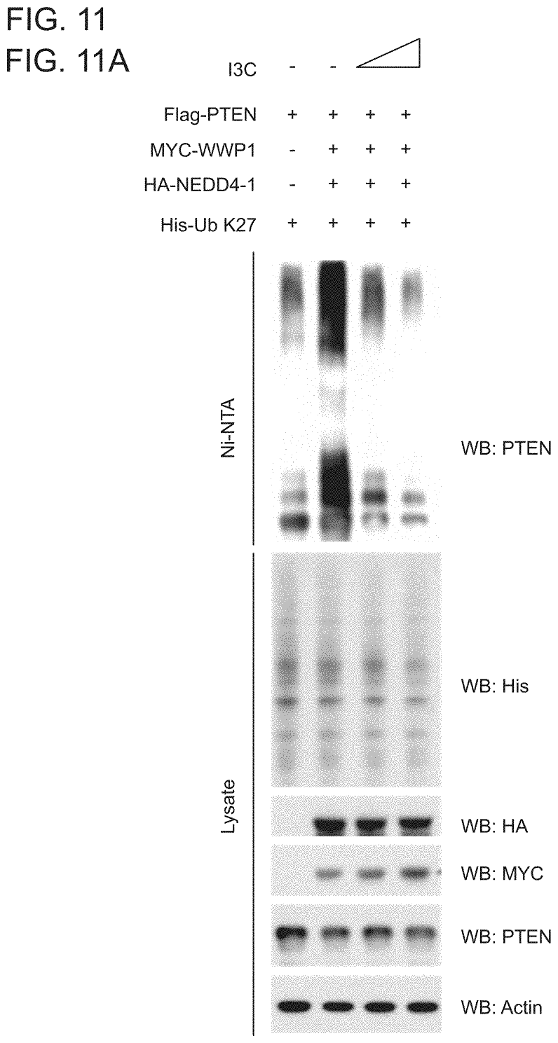

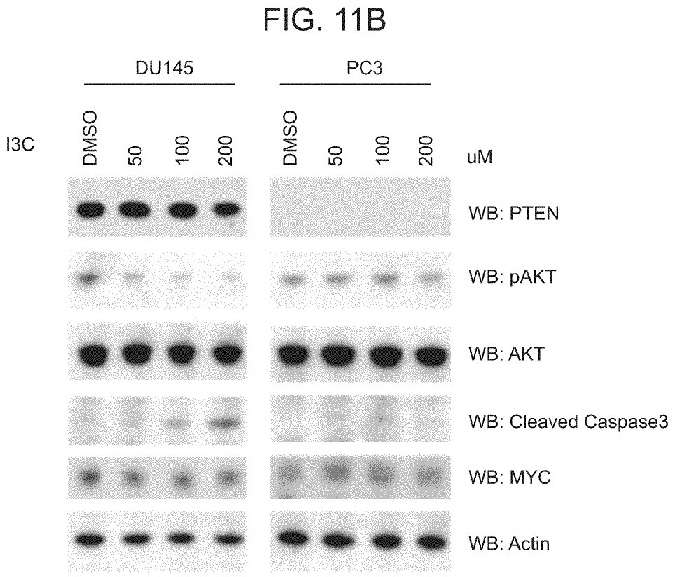

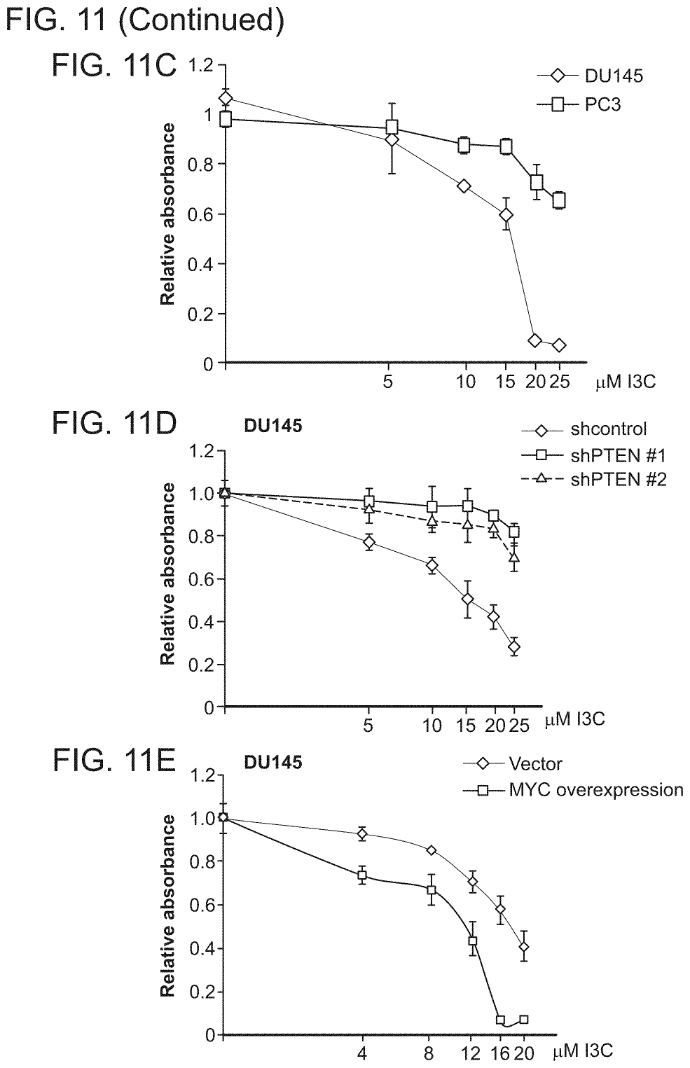

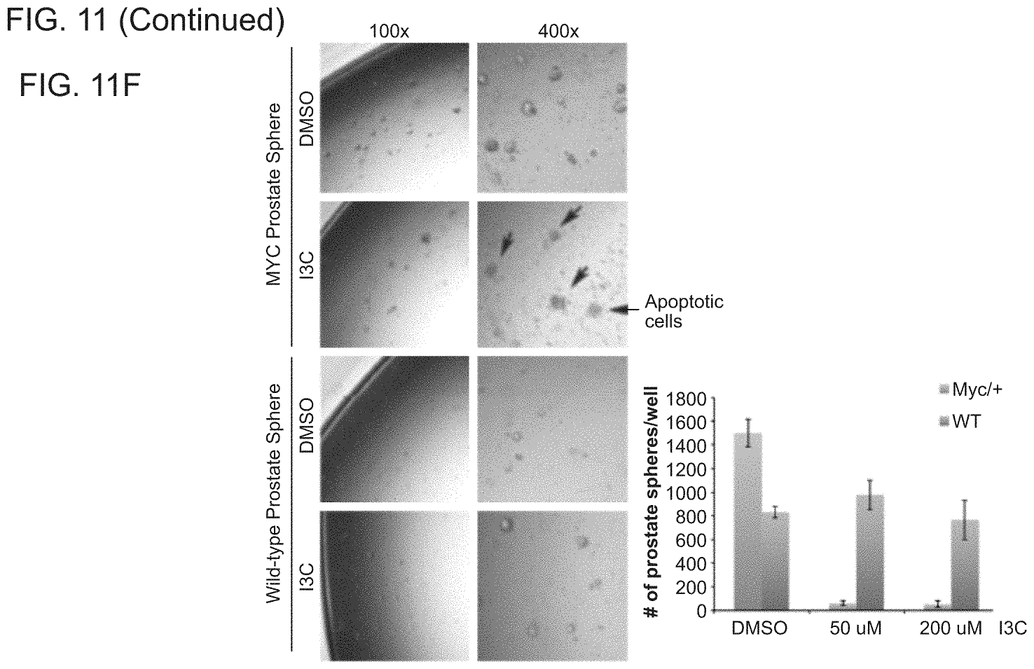

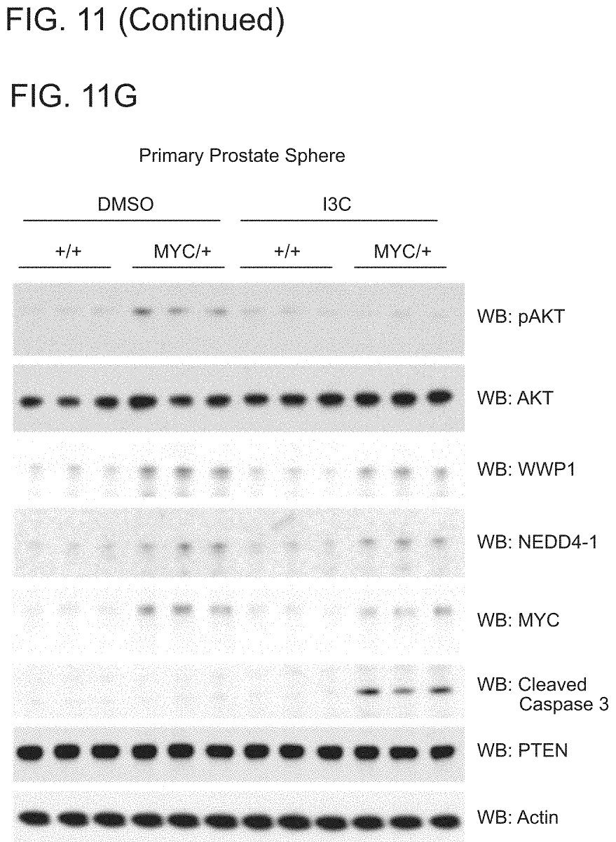

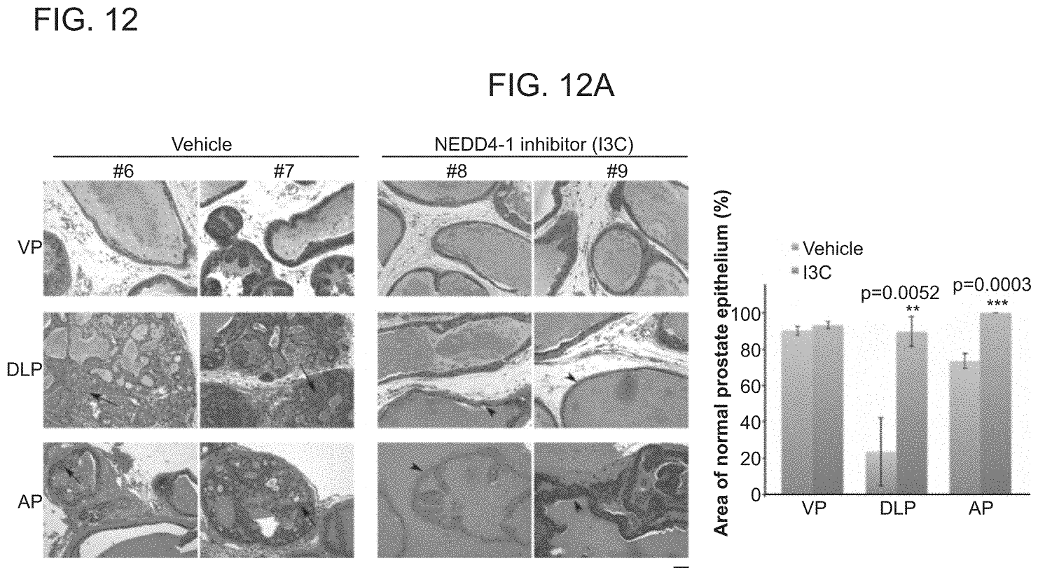

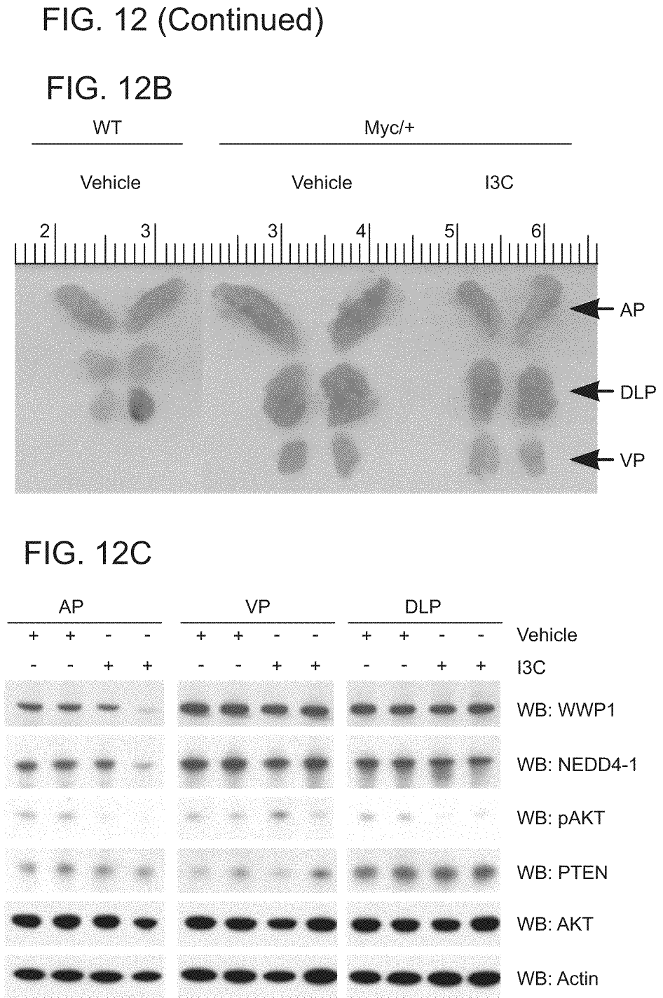

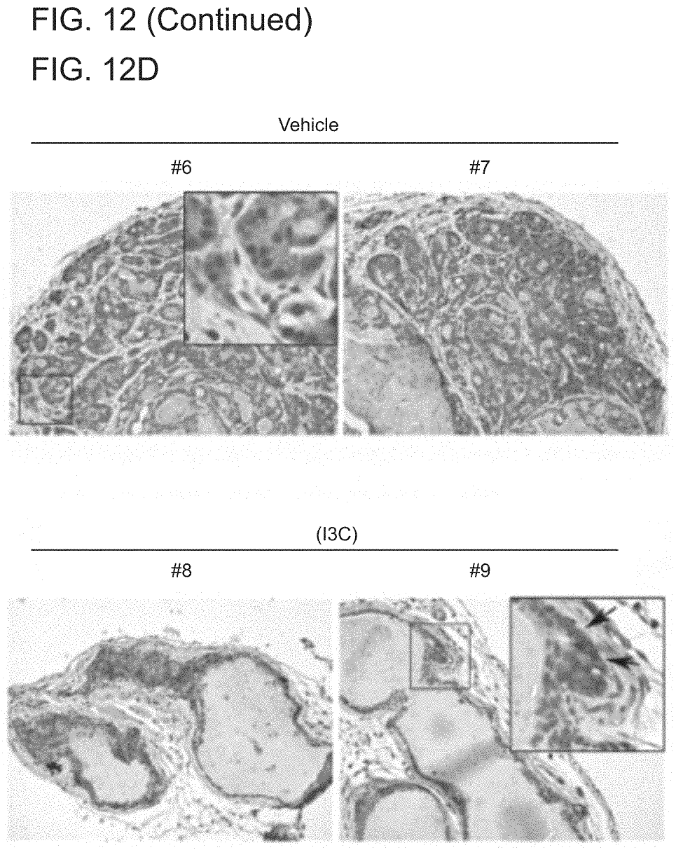

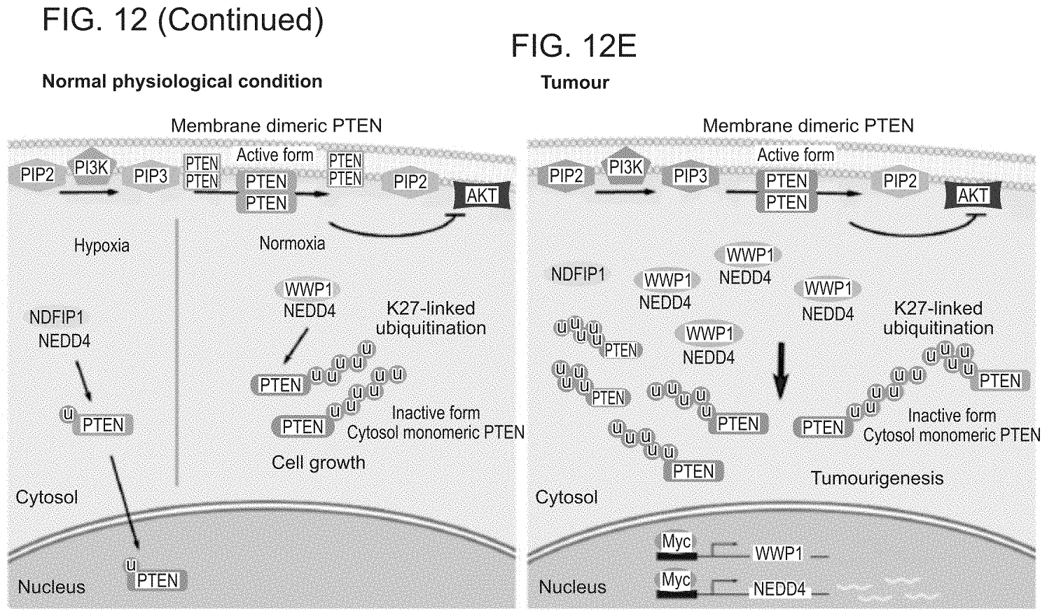

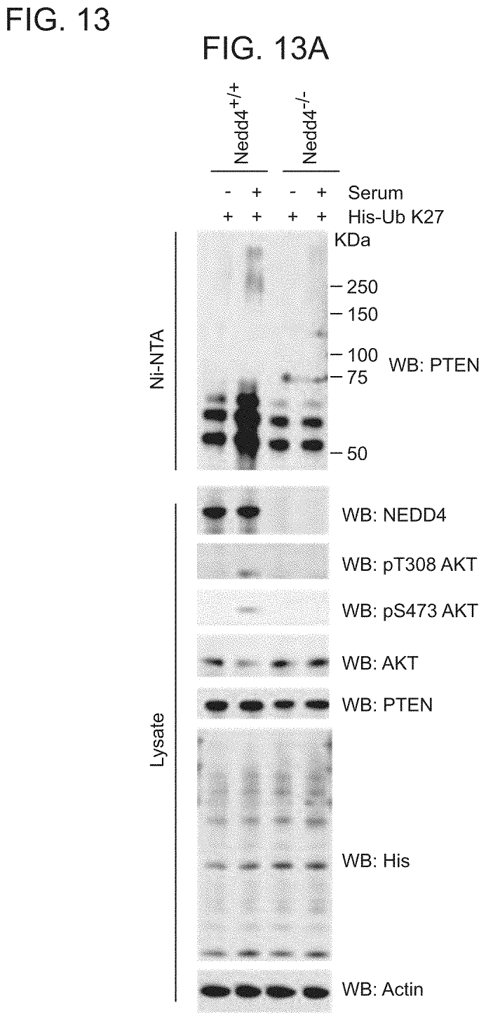

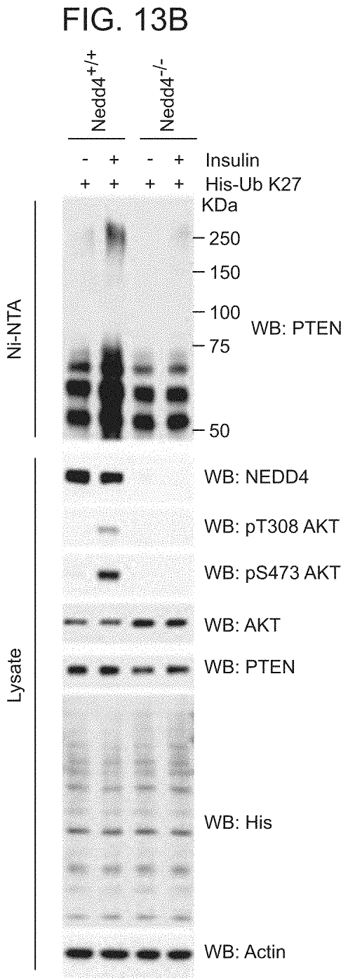

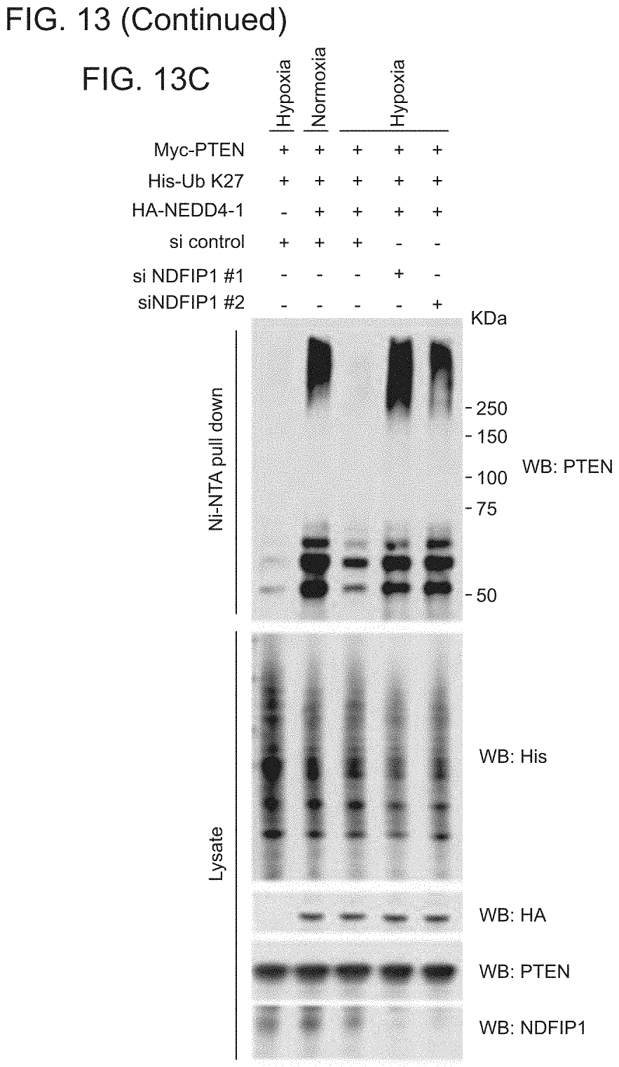

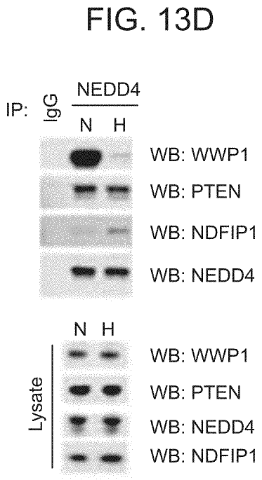

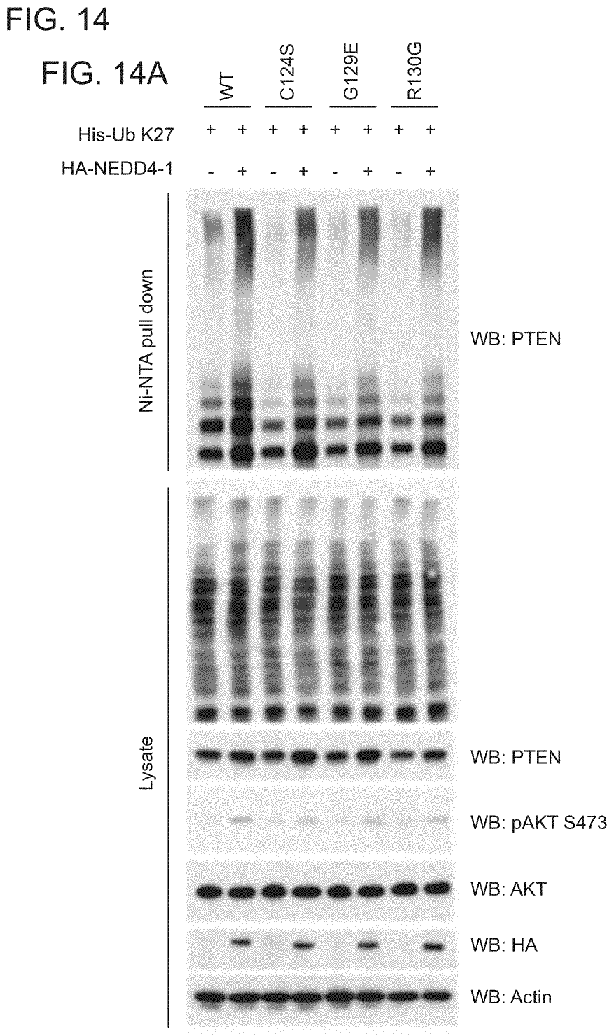

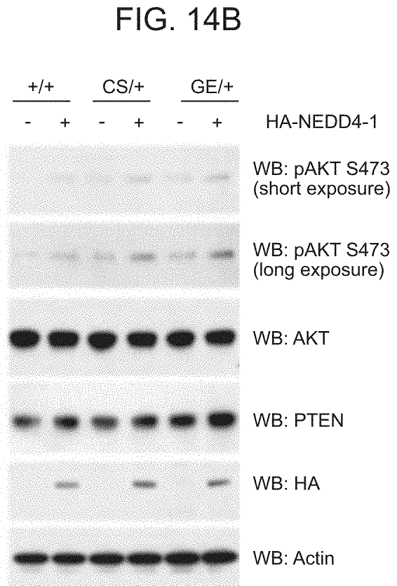

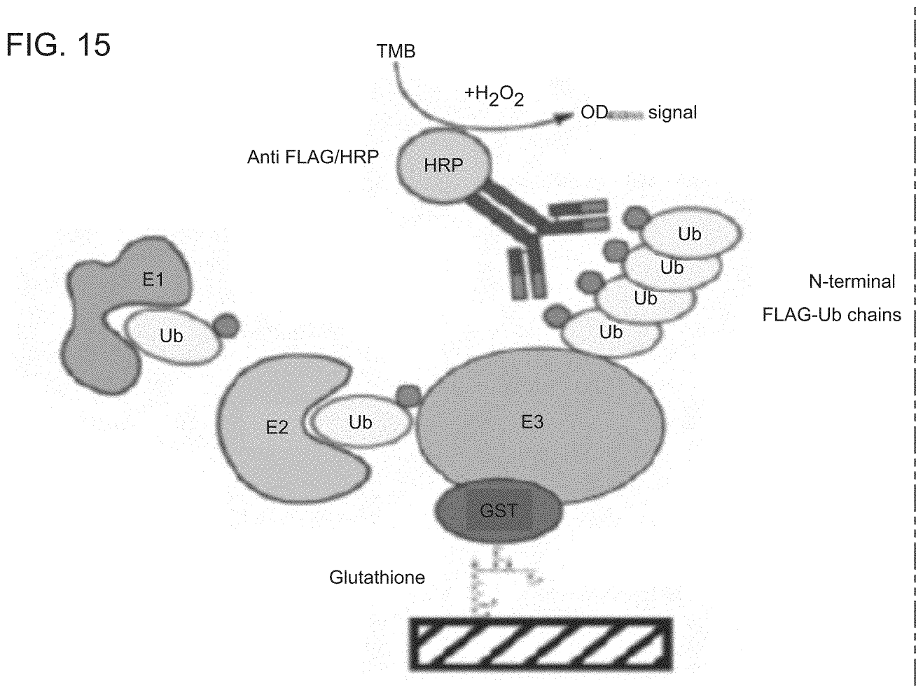

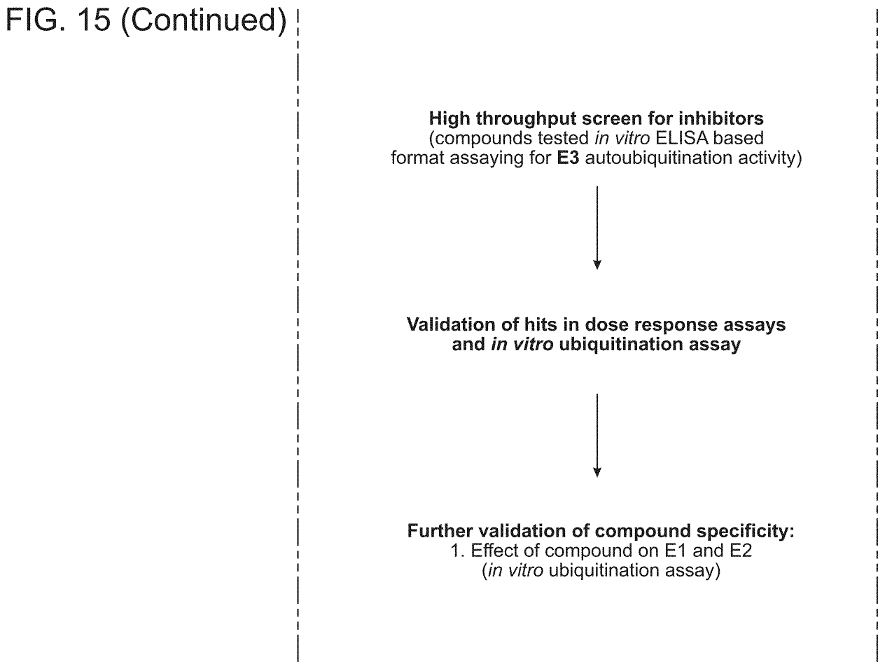

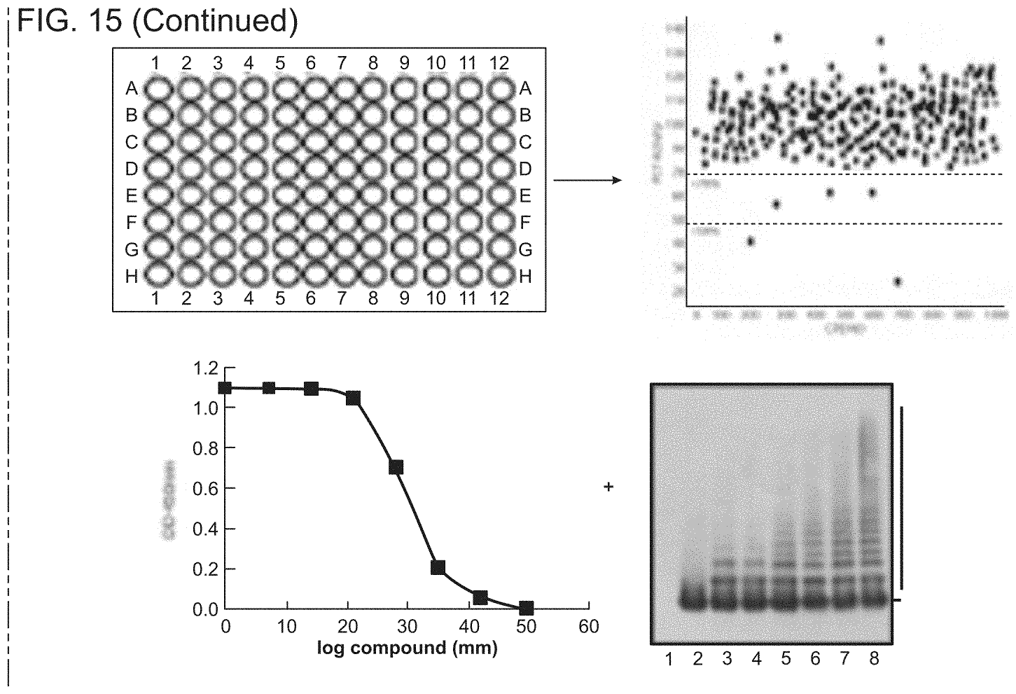

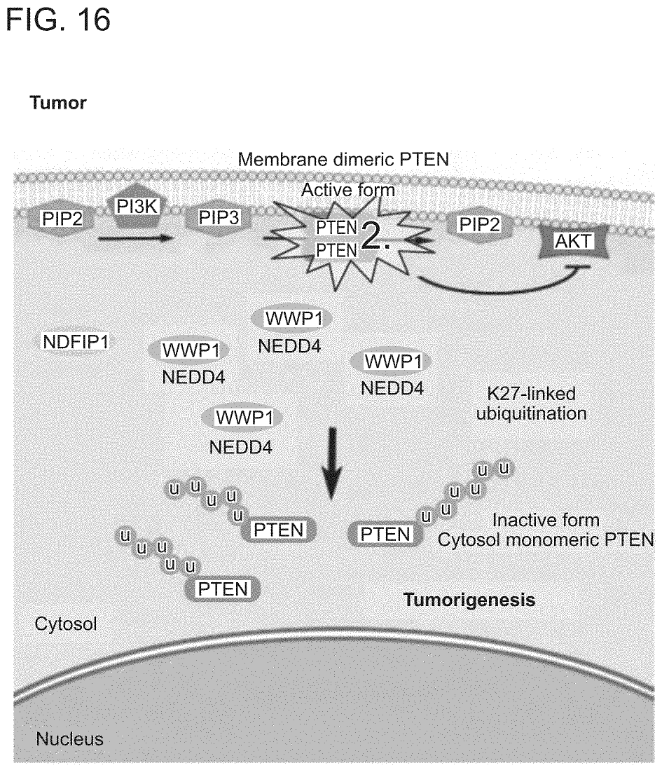

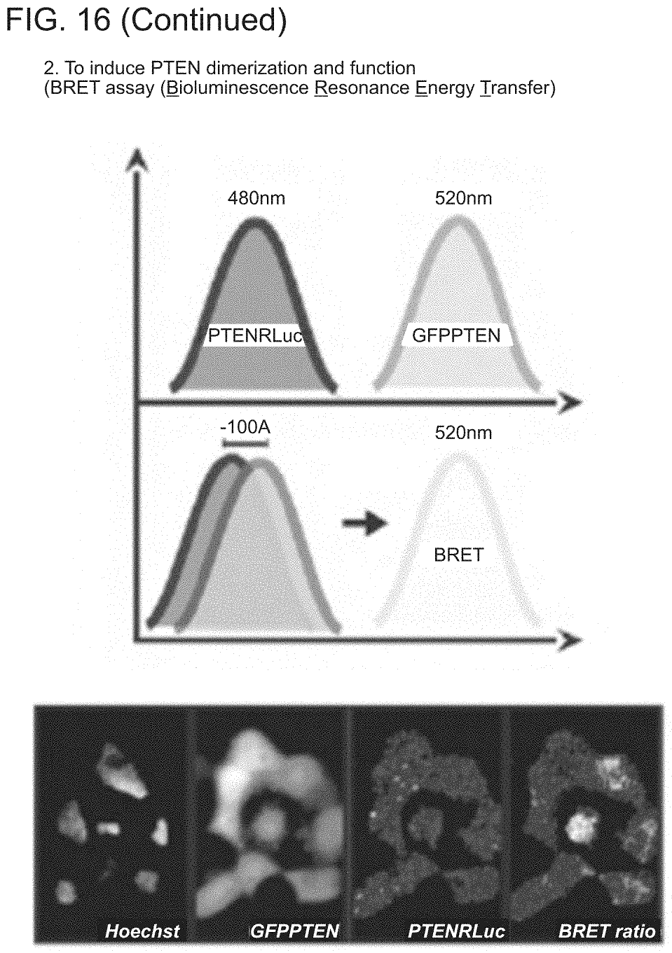

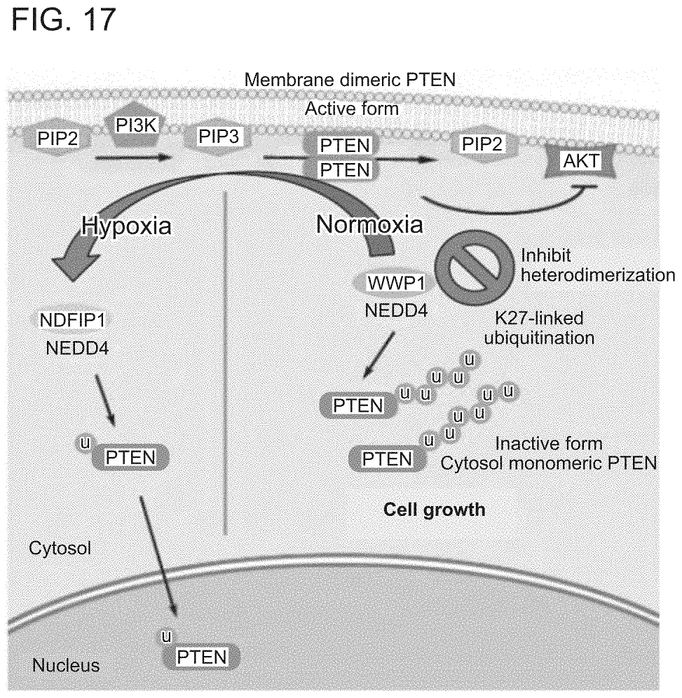

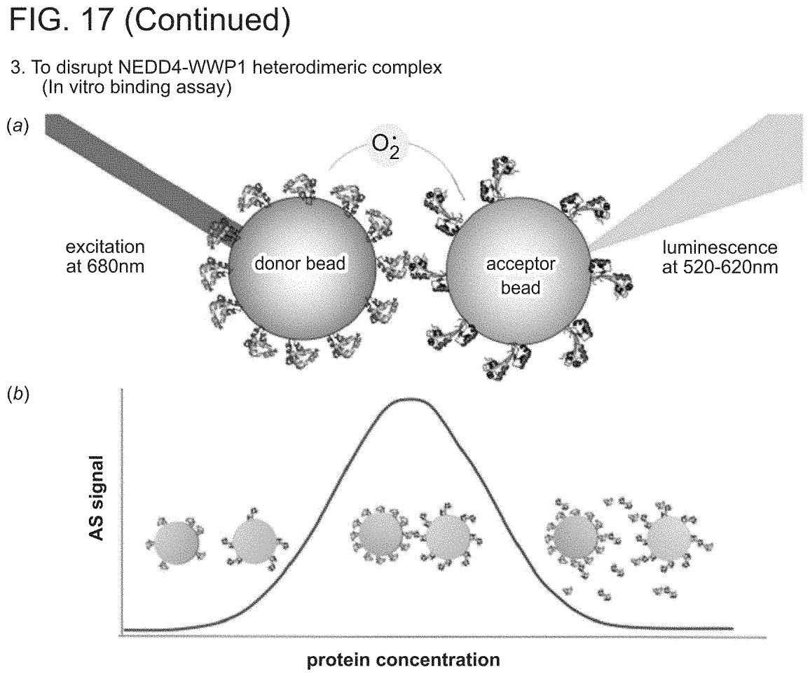

FIG. 6J is a graph showing the effects of WWP1/NEDD4-1 on MYC-induced colony forming activity in soft agar. The colony numbers are quantified and presented as mean±SD (***P<0.0005, **P<0.005, n=3).

FIG. 6K is a graph showing the results of an apoptosis assay of DU145 cells stably expressing MYC and/or WWP1/NEDD4-1 shRNAs. The percentage of apoptotic cells was quantified by the ratio of Sub-G1 phase. (***P<0.0005, **P<0.005, n=3).

FIG. 6L is a table showing the correlation of NEDD4-1 expression and inverse correlation of PTEN membrane recruitment in human CaP specimens.

FIG. 6M is a table showing positive correlation of NEDD4-1 expression and inverse correlation of PTEN membrane recruitment with prostate tumour grade.

FIG. 6N is a table showing significant correlation of increased NEDD4-1 expression and loss of PTEN membrane recruitment with disease progression. Statistical significance was determined by a Pearson chi-squared test in (1-n).

FIG. 6O is an expression analysis between NEDD4-1 and WWP1 in TCGA (n=419) and MSKCC (n=85) data set of human prostate adenocarcinoma. NEDD4-1 mRNA is positively correlated with WWP1 mRNA in both data sets.

FIG. 7A-7D shows the cell-cycle profile of DU145 cells stably expressing indicated constructs.

FIG. 7A consists of four graphs and provides an analysis of cell-cycle profile of DU145 cells stably expressing indicated by staining with propidium iodide (PI), and then followed by FACS analysis. The results were analysed by the FlowJo software.

FIG. 7B shows total, membrane, and soluble fractions isolated from different CaP cell lines were analysed by western blot. EGFR serves as a membrane marker, and Actin as the internal control.

FIG. 7C provides an immunohistochemical analysis of NEDD4-1 and PTEN. Representative immunohistochemistry assay (IHC) results for NEDD4-1 and PTEN in human CaP specimens. Bar, 20 μm.

FIG. 7D provides an expression analysis between MYC and WWP1 or NEDD4-1 in TCGA data set of human prostate adenocarcinoma. MYC mRNA is positively correlated with WWP1 or NEDD4-1 mRNA.

FIG. 8 shows WWP1 is co-amplified with MYC in human prostate tumors—Co-amplification analysis between MYC and WWP1 in TCGA data set of human prostate adenocarcinoma.

FIGS. 9A-9D shows the effects of WWP1 involved in Myc-driven prostate cancer.

FIG. 9A provides an image of an H&E staining of mouse prostate and gross anatomy of representative mice urogenital tract. The mice at 3 months of age were analyzed (n=5 per genotype). scale bar, 50 μm.

FIG. 9B provides images of the gross anatomy of representative mice urogenital tract. The mice at 3 months of age were analyzed (n=5 per genotype). scale bar, 50 μm.

FIG. 9C provides western blot analysis of the dorsal lateral prostates (DLPs) from Hi-Myc; Wwp1^{+/+} and Hi-Myc; Wwp1^{+/-} mice.

FIG. 9D provides images of the dorsal lateral prostates (DLPs) from Hi-Myc; Wwp1^{+/+} and Hi-Myc; Wwp1^{+/-} mice analyzed by PTEN staining. Bar, 50 μm.

FIGS. 10A-10D shows the in silico modeling of the predicted interactions of Indole-3-Carbinol (I3C) with the HECT domain of WWP1 and the effects of Indole-3-Carbinol (I3C), a WWP1 inhibitor, on cell growth and PTEN subcellular localization.

FIG. 10A provides in silico modeling of the predicted interactions of I3C with the HECT domain of WWP1.

FIG. 10B provides the MST analysis of I3C towards WWP1.

FIG. 10C provides growth curves of Wwp1^{+/+} and Wwp1^{-/-} MEFs treated with 100 μM I3C. Representative pictures of the cells at Day 3 are shown below.

FIG. 10D provides a panel of micrographs showing the effects of I3C on PTEN subcellular localization in DU145 cells. DU145 cells treated with 20 μM I3C for 4 Days. Confocal images of DU145 cells treated with/without I3C were stained with DAPI and the indicated antibodies. Bar, 20 μm.

FIGS. 11A-11G shows the therapeutic potential for targeting WWP1/NEDD4-1 E3 in vitro.

FIG. 11A shows the effects of I3C on WWP1/NEDD4-1 mediated PTEN K27-linked poly-ubiquitination. DU145 cells expressing indicated plasmids were treated with 10 μM or 20 μM of I3C for 4 Days. The ubiquitinated PTEN was pulled down under denaturing conditions by Ni-NTA agarose and analysed by the western blot.

FIG. 11B provides an analysis of effects of I3C on PTEN/AKT pathway in DU145 or PC3 cells by western blot analysis. Cells were treated with different doses of I3C for 3 Days, and followed by western blot analysis.

FIG. 11C provides growth curves of DU145 (PTEN competent) or PC3 cells (PTEN null) treated with various doses of I3C for 5 Days, and then assayed with colorimetric MTT assay (three experiments).

FIG. 11D provides growth curves of DU145 cells, stably expressing vector control or two independent shRNAs, treated with various doses of I3C for 5 Days, and then assayed with colorimetric MTT assay (three experiments).

FIG. 11E provides growth curves of DU145 cells, stably expressing MYC or vector control, treated with various doses of I3C for 5 Days, and then assayed with colorimetric MTT assay (three experiments).

FIG. 11F are micrographs showing an analysis of I3C on prostate sphere forming ability of WT or Hi-Myc prostate prostatic epithelial cells. Representative images of spheres at P1 passage treated with 10 μM I3C for 3 Day (left). Bar, 20 μm. The sphere numbers were quantified and presented as mean±SD (***P<0.0005, **P<0.005, n=3) (right).

FIG. 11G is a Western blot analysis of prostate spheres at P1 passage treated with 20 μM I3C for 3 Days.

FIGS. 12A-12E shows the therapeutic targeting WWP1/NEDD4-1 E3 in vivo.

FIGS. 12A and 12B show hematoxylin and eosin (H&E) staining of mouse prostate and gross anatomy of representative Hi-Myc urogenital tract. Quantification of percentage of normal epithelium in Hi-Myc mice treated with vehicle or I3C at 6 months as shown in (A) (right). The mice at 5 months of age were treated I.P. with I3C (20 mg/kg), three times a week for 1 month starting on day zero. (n=4 per genotype) scale bar, 50 μM.

FIG. 12C provides a Western blot analysis of dorsal lateral prostate (DLP) lysates from Hi-Myc mice as in (A).

FIG. 12D shows DLPs from Hi-Myc mouse prostates treated with vehicle or I3C (20 mg/kg) were analysed by PTEN staining. The boxed areas are enlarged three fold to show the subcellular localization of PTEN on the right. Arrow indicates the PTEN plasma membrane localization. Bar, 50 μm.

FIG. 12E provides a model for WWP1/NEDD4-1 mediated PTEN K27-linked poly-ubiquitination in cell growth, tumour development and progression. Deregulated MYC overexpression or MYC amplification promotes WWP1/NEDD4-1 expression, and in turn triggers PTEN K27-linked poly-ubiquitination. Aberrant K27-linked poly-ubiquitination suppresses PTEN dimerization, plasma membrane recruitment and tumour suppressive function, leading to the tumour initiation and progression.

FIGS. 13A-13D shows the regulation of PTEN K27-linked poly-ubiquitination under different physiological stimuli.

FIG. 13A and FIG. 13B shows an analysis of PTEN K27-linked ubiquitination upon growth factor stimulation. Nedd4^{+/+} and Nedd4^{-/-} MEFs were serum-starved for 3 hours and were treated with or without 10% FBS/200 ng/ml insulin for 15 minutes; lysates were collected for Ni-NTA pull down, followed by western blot analysis.

FIG. 13C shows an analysis of NEDD4-1 mediated PTEN ubiquitination status under normoxic or hypoxic conditions. 293 cells expressing indicated constructs were cultured in hypoxic or normoxic conditions for 16 hours. Total cell lysates were pulled down by Ni-NTA beads under denaturing conditions, followed by western blot to analyse PTEN ubiquitination.

FIG. 13D is a Western blot showing that NEDD4-1 switches its interaction proteins under different physiological conditions. PWRE-1 cells were cultured in normoxic or hypoxic conditions for 16 hours. Total cell lysates were immunoprecipitated with anti-NEDD4 antibody, and then examined by western blot with indicated antibodies.

FIG. 14A and FIG. 14B show that NEDD4-1 robustly induces K27-linked poly-ubiquitination and AKT activation in PTEN having cancer-associated mutations.

FIG. 14A shows an analysis of PTEN K27-linked poly-ubiquitination in PC3 cells expressing indicated PTEN WT or its cancer-associated mutants (C124S, G129E, and R130G) along with NEDD4-1.

FIG. 14B shows a Western blot analysis of AKT activation in primary PTEN^{+/+}, PTEN^{CS/+}, and PTEN^{GE/+} MEFs expressing HA-NEDD4-1.

FIG. 15 depicts an ELISA-based assay useful for identifying inhibitors of NEDD4, and/or WWP1-mediated ubiquitination.

FIG. 16 depicts a fluorescence-based assay useful for identifying inhibitors of PTEN dimerization.

FIG. 17 depicts a fluorescence-based assay useful for identifying inhibitors of NEDD4-1/WWP1 heterodimer formation.

FIG. 18 is a table showing a list of PTEN associated proteins.

### DETAILED DESCRIPTION OF THE INVENTION

The invention generally provides methods of treating cancer (e.g., bladder cancer, breast cancer, colon adenocarcinoma, gastric adenocarcinoma, prostate cancer, liver cancer), the method comprising administering to a subject having a cancer, an effective amount of an agent that inhibits the expression or activity of neural precursor cell expressed developmentally down-regulated protein 4 (NEDD4), and an agent that inhibits the expression or activity of WW domain-containing protein-1 (WWP1).

The invention is based, at least in part, on the discovery that K27-linked poly-ubiquitination suppresses PTEN dimerization, membrane recruitment and function. As reported in more detail below, WWP1/NEDD4-1 E3 ligases were found to interact with PTEN and were essential to cooperatively catalyze this non-degradative modification. WWP1 and NEDD4-1 were discovered to be both direct MYC target genes and were important for its tumorigenic function. Analysis of human tumours reveals that the concomitant overexpression of MYC/NEDD4-1/WWP1 correlated with disease progression and PTEN membrane displacement. Importantly, it was demonstrated that the pharmacological inhibition of WWP1/NEDD4-1 triggered PTEN reactivation and a potent suppression of MYC-driven tumorigenesis both in vitro and in vivo. These findings therefore unravel the oncogenic role for K27-linked PTEN poly-ubiquitination, and a therapeutic strategy for the treatment of MYC-driven cancers through PTEN reactivation.

Accordingly, the invention provides methods of using agents (e.g., polypeptides, inhibitory nucleic acids, and small molecules) that inhibit NEDD4-1 and/or WWP1 expression or activity for the treatment of cancer (e.g., bladder cancer, breast cancer, colon adenocarcinoma, gastric adenocarcinoma, prostate cancer, liver cancer).

Inhibitory Nucleic Acids

Inhibitory nucleic acid molecules are those oligonucleotides that inhibit the expression or activity of NEDD4-1 or WWP1. Such oligonucleotides include single and double stranded nucleic acid molecules (e.g., DNA, RNA, and analogs thereof) that bind a nucleic acid molecule that encodes a NEDD4-1 or WWP1 polypeptide (e.g., antisense molecules, siRNA, shRNA), as well as nucleic acid molecules that bind directly to the polypeptide to modulate its biological activity (e.g., aptamers). Inhibitory nucleic acid molecules described herein are useful for the treatment of cancer (e.g., (e.g., bladder cancer, breast cancer, colon adenocarcinoma, gastric adenocarcinoma, prostate cancer, liver cancer).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as therapeutics to treat cancer.

The inhibitory nucleic acid molecules of the present invention may be employed as double-stranded RNAs for RNA interference (RNAi)-mediated knock-down of expression. In one embodiment, expression of NEDD4-1 polypeptide and/or WWP1 polypeptide is reduced in a subject having cancer. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a nucleic acid molecule in vivo (e.g., a nucleic acid encoding NEDD4-1 or WWP1). The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Alternatively, expression of NEDD4-1, WWP1, or both, may be inhibited, or silenced by introducing vectors encoding Clustered regularly interspaced short palindromic repeats (CRISPR)/Cas9 nuclease engineered to target NEDD4-1, WWP1, or both.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

Delivery of Polynucleotides

Naked polynucleotides, or analogs thereof, are capable of entering mammalian cells and inhibiting expression of a gene of interest (e.g., a NEDD4-1 or WWP1 polynucleotide). Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

Small Molecule Inhibitors

The invention provides small molecules capable of inhibiting NEDD4-1 and/or WWP1 activity that are useful for the treatment of cancer. Examples of compounds suitable as a NEDD4-1 inhibitor include 4-(4-chlorobenzoyl) piperazin-1-yl) (4-(phonoxymethyl) phenyl) methanone.

Another example of a compound suitable as a NEDD4-1 inhibitor is indole-3-carbinol (I3C). The structure of I3C is shown below:

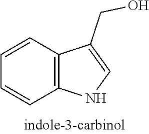 [SMILES: OCc1c[nH]c2ccccc12]

Another example of compounds suitable as NEDD4-1 inhibitors are the compounds listed in U.S. Patent Application No. US20140179637 A1 (incorporated by reference in its entirety).

Therapeutic Methods

The methods and compositions provided herein can be used to treat or prevent progression of a cancer (e.g., bladder cancer, breast cancer, colon adenocarcinoma, gastric adenocarcinoma, prostate cancer, liver cancer). In general, an effective amount of at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) can be administered therapeutically and/or prophylactically.

Treatment will be suitably administered to subjects, particularly humans, suffering from, having, susceptible to, or at risk of developing such cancer. Determination of those subjects “at risk” can be made by any objective or subjective determination by a diagnostic test or opinion of a subject or health care provider (e.g., genetic test, enzyme or protein marker, family history, and the like). Identifying a subject in need of such treatment can be in the judgment of a subject or a health care professional and can be subjective (e.g. opinion) or objective (e.g. measurable by a test or diagnostic method).

In some aspects, the effective amount of at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) may be administered in combination with one or more of any other standard anti-cancer therapies. For example, an agent as described herein may be administered in combination with standard chemotherapeutics. Methods for administering combination therapies (e.g., concurrently or otherwise) are known to the skilled artisan and are described for example in Remington's Pharmaceutical Sciences by E. W. Martin.

Chemotherapeutic Agents

The invention further provides for the use of conventional chemotherapeutics in combination with an agent that inhibits NEDD4-1 or WWP1 expression or activity. Chemotherapeutic agents suitable for use in the methods of the present invention include, but are not limited to alkylating agents. Without intending to be limited to any particular theory, alkylating agents directly damage DNA to keep the cell from reproducing. Alkylating agents work in all phases of the cell cycle and are used to treat many different cancers, including leukemia, lymphoma, Hodgkin disease, multiple myeloma, and sarcoma, as well as cancers of the lung, breast, and ovary.

Alkylating agents are divided into different classes, including, but not limited to: (i) nitrogen mustards, such as, for example mechlorethamine (nitrogen mustard), chlorambucil, cyclophosphamide (Cytoxan®), ifosfamide, and melphalan; (ii) nitrosoureas, such as, for example, streptozocin, carmustine (BCNU), and lomustine; (iii) alkyl sulfonates, such as, for example, busulfan; (iv) riazines, such as, for example, dacarbazine (DTIC) and temozolomide (Temodar®); (v) ethylenimines, such as, for example, thiotepa and altretamine (hexamethylmelamine); and (v) platinum drugs, such as, for example, cisplatin, carboplatin, and oxalaplatin.

Pharmaceutical Compositions

The present invention features compositions useful for treating cancer. The methods include administering to a subject having a cancer, an effective amount of at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) in a physiologically acceptable carrier.

Typically, the carrier or excipient for the composition provided herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like. The administration may be by any suitable means that results in a concentration of the therapeutic that, combined with other components, is effective in ameliorating, reducing, or stabilizing the disease symptoms in a subject. The composition may be administered systemically, for example, formulated in a pharmaceutically-acceptable buffer such as physiological saline. Preferable routes of administration include, for example, subcutaneous, intravenous, interperitoneally, intramuscular, intrathecal, or intradermal injections that provide continuous, sustained levels of the agent in the patient. The amount of the therapeutic agent to be administered varies depending upon the manner of administration, the age and body weight of the patient, and with the clinical symptoms of the cancer. Generally, amounts will be in the range of those used for other agents used in the treatment of cancer, although in certain instances lower amounts will be needed because of the increased specificity of the agent. A composition is administered at a dosage that ameliorates or decreases effects of the cancer as determined by a method known to one skilled in the art.

The therapeutic or prophylactic composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intrathecally, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target a disease using carriers or chemical derivatives to deliver the therapeutic agent to a particular cell type. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cardiac dysfunction or disease, the composition may include suitable parenterally acceptable carriers and/or excipients. The active therapeutic agent(s) (e.g., at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) described herein) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

Kits

The invention provides kits for the treatment or prevention of cancer. In some embodiments, the kit includes a therapeutic or prophylactic composition containing at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) in unit dosage form. In other embodiments, the kit includes at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) in unit dosage form in a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a pharmaceutical composition of the invention is provided together with instructions for administering the pharmaceutical composition to a subject having or at risk of contracting or developing cancer. The instructions will generally include information about the use of the composition for the treatment or prevention of cancer. In other embodiments, the instructions include at least one of the following: description of the therapeutic/prophylactic agent; dosage schedule and administration for treatment or prevention of cancer or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

Murine Platform for Screening Therapies

The invention provides a method of identifying a therapeutic agent for a subject having a cancer characterized by one or more defined genetic lesions (e.g., an over expression of cMYC). The method involves obtaining a neoplastic cell from a mouse having one or more of the same defined genetic lesions; culturing the neoplastic cell in vitro to obtain one or more neoplastic cells or cancer organoids; implanting the neoplastic cell or cancer organoid into an immune competent syngeneic mouse; administering one or more candidate agents to the syngenic mouse; and assaying the biological response of the neoplastic cell, organoid or syngeneic mouse to the candidate agent.

The invention further provides methods for characterizing therapies in immunocompromised mice that are implanted with human tumor cell lines or primary human tumors (PDX models). In particular embodiments, an implanted tumor constitutively overexpresses MYC, is engineered to over-express MYC, or is engineered to have reduced (e.g. via shRNA knockdown) MYC. Immunocompromised mice generally lack adaptive immune system components, but have relatively intact innate immune systems. Therefore, upon tumor formation, infiltration of mouse MDSCs is assessed along with their phenotypic characteristics (immunosuppressive markers, cell surface markers, immunosuppressive potency). A similar approach is taken with mouse tumor lines in syngenic hosts. In either xenograft or syngenic models, tumor cell lines overexpressing human or mouse MYC are assessed. Such mice are used to assess the biological response to at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1). For example, the effects of at least one agent selected from the group consisting of: an agent that inhibits the expression or activity of neural precursor cell expressed developmentally down-regulated protein 4 (NEDD4-1), and an agent that inhibits the expression or activity of WW domain-containing protein-1 (WWP1) is evaluated by assaying, tumor growth, and/or murine survival.

In another embodiment, mice are implanted with organoids that either endogenously express MYC or are engineered to do so. Methods for generating organoids are known in the art and described, for example, by Boj et al., Cell; 160: 324-338, 2015; Gao et al., Cell; 159: 176-187, 2014; Linde et al., PLoS ONE; 7(7): e40058, 2012. In another embodiment, organoids are maintained in co-culture with autologous PBMC using tumor tissue and PBMCs from the same human patient.

In Vitro Screening

Referring to FIG. 15, FIG. 16, and FIG. 17, the present invention provides in vitro screening platforms to identify therapeutic agent for a subject having a cancer. For example, FIG. 15 depicts an ELISA-based assay useful for identifying inhibitors of NEDD4-1, and/or WWP1-mediated ubiquitination. (See Example 1). FIG. 16 depicts a fluorescence-based assay useful for identifying inhibitors of PTEN dimerization. (See Example 4). FIG. 17 depicts a fluorescence-based assay useful for identifying inhibitors of NEDD4-1/WWP1 heterodimer formation.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow. The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1—WWP1/NEDD4-1 E3 Ligases Mediate PTEN K27-Linked Poly-Ubiquitination

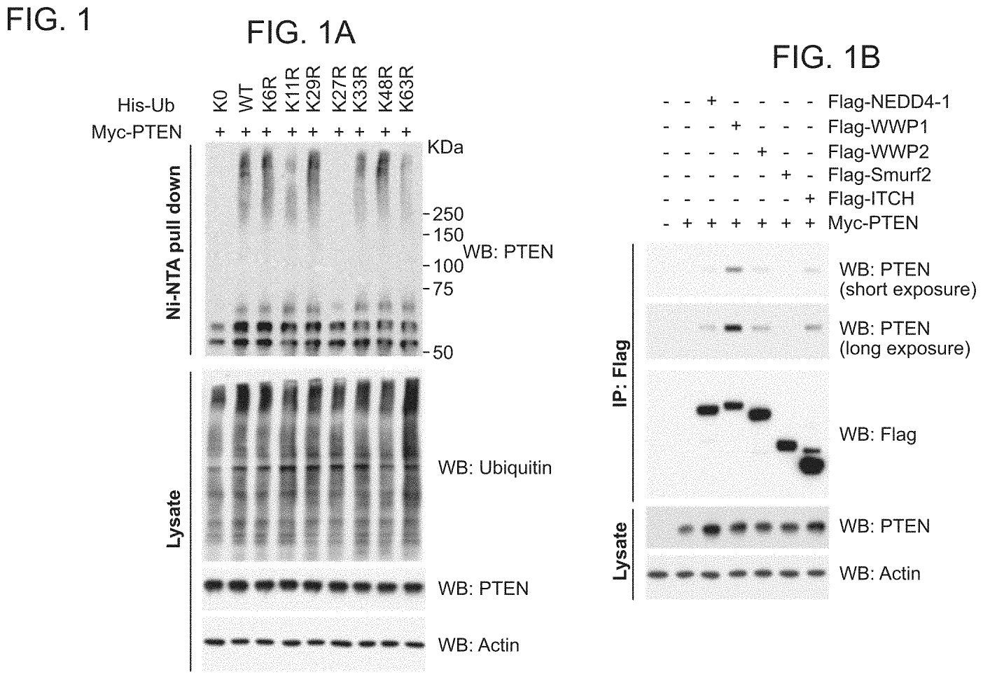

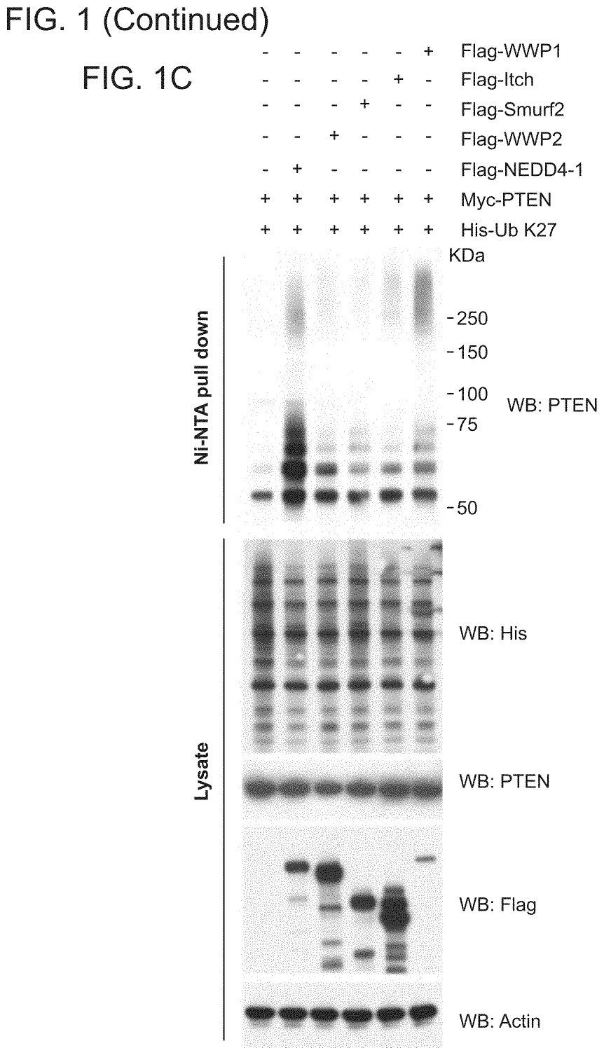

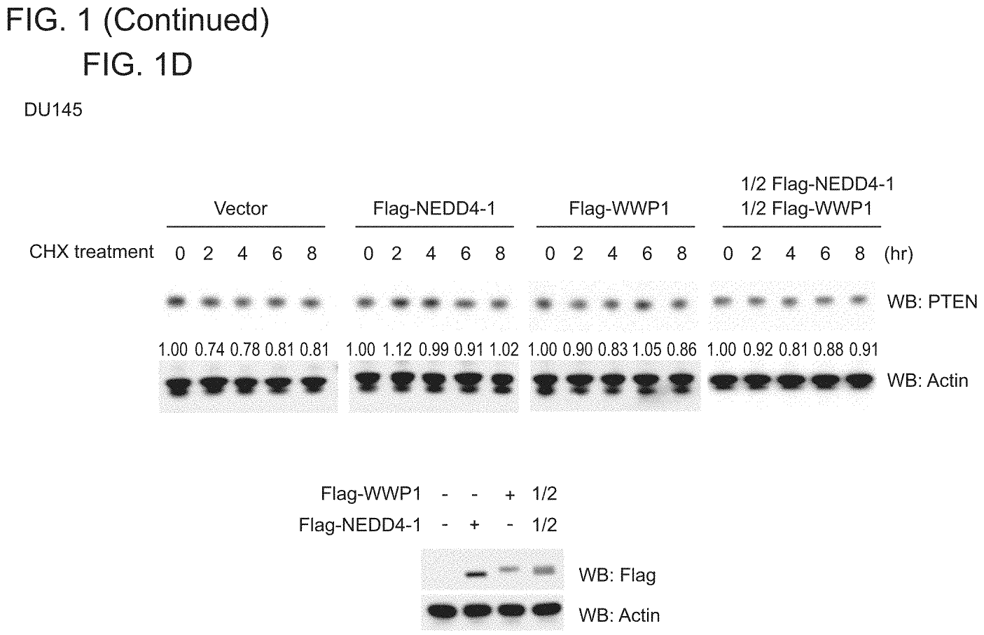

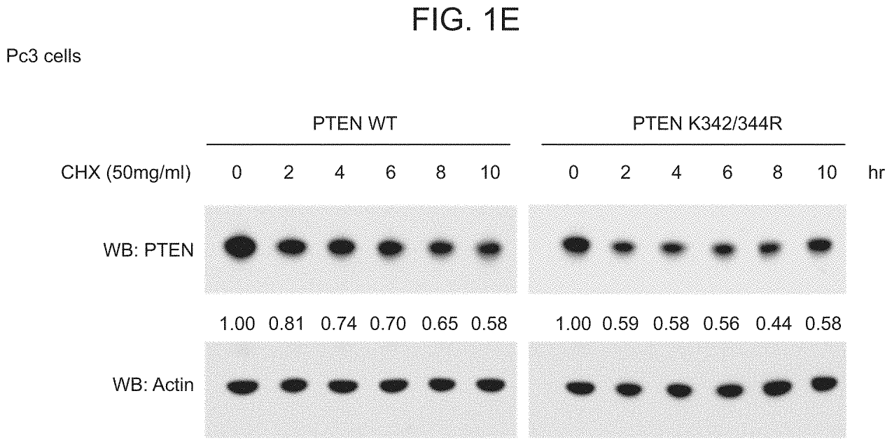

The studies presented in this example show that PTEN was ubiquitinated, even in the absence of the proteasome inhibitor MG132 (FIG. 1A), suggesting to test whether ubiquitination would control PTEN functions other than its turnover. The identity of which ubiquitin chain was formed on PTEN was then investigated. By mutating each lysine residue of ubiquitin individually, it was surprisingly found that at a steady state only the K27R ubiquitin mutant markedly impaired PTEN poly-ubiquitination, suggesting that PTEN is predominantly modulated by the K27-linked ubiquitin chain, but not the other lysine-linked ubiquitin chains (FIG. 1A).

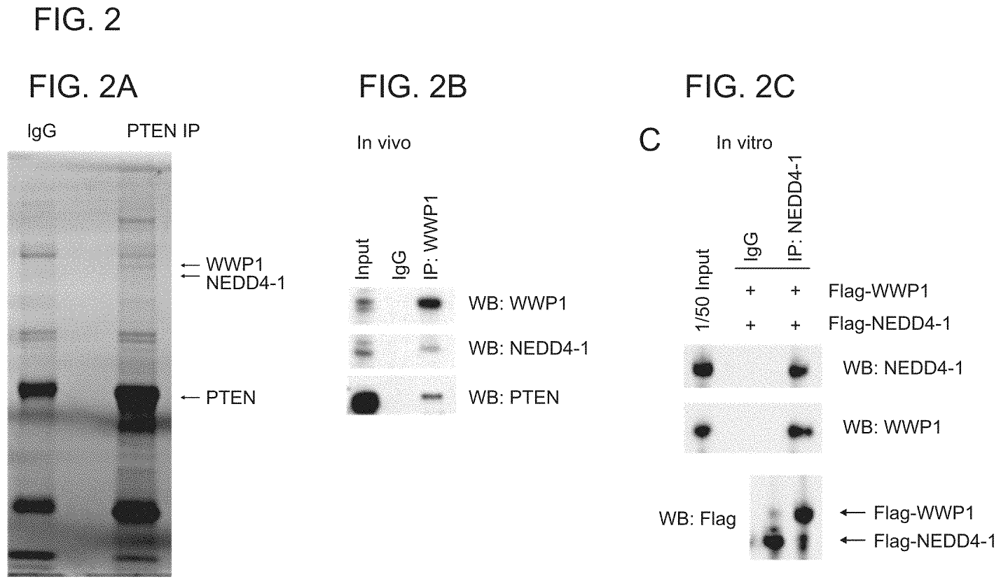

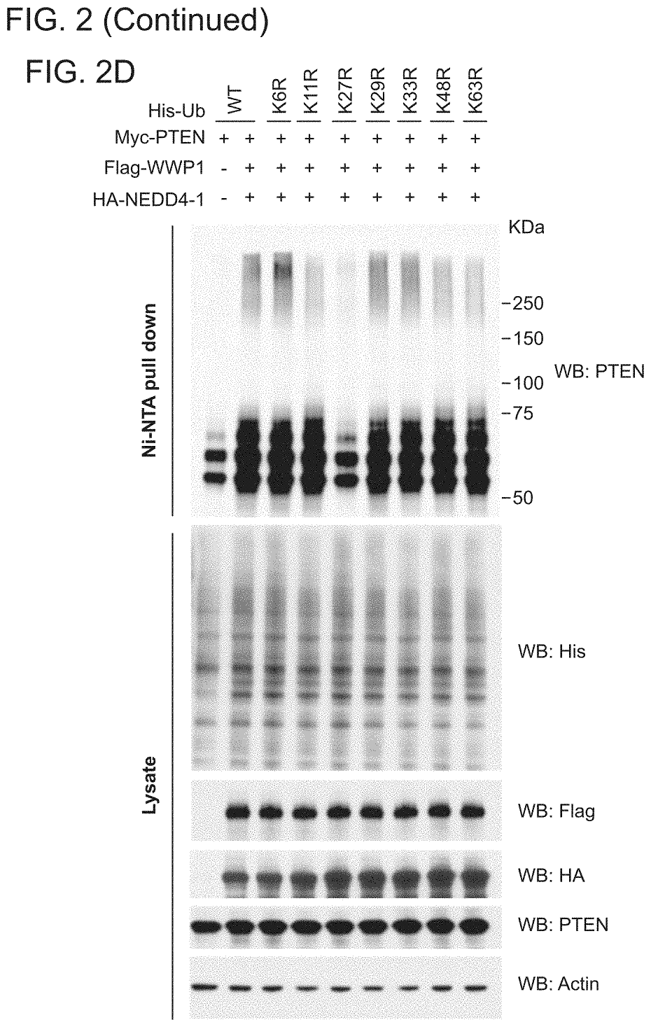

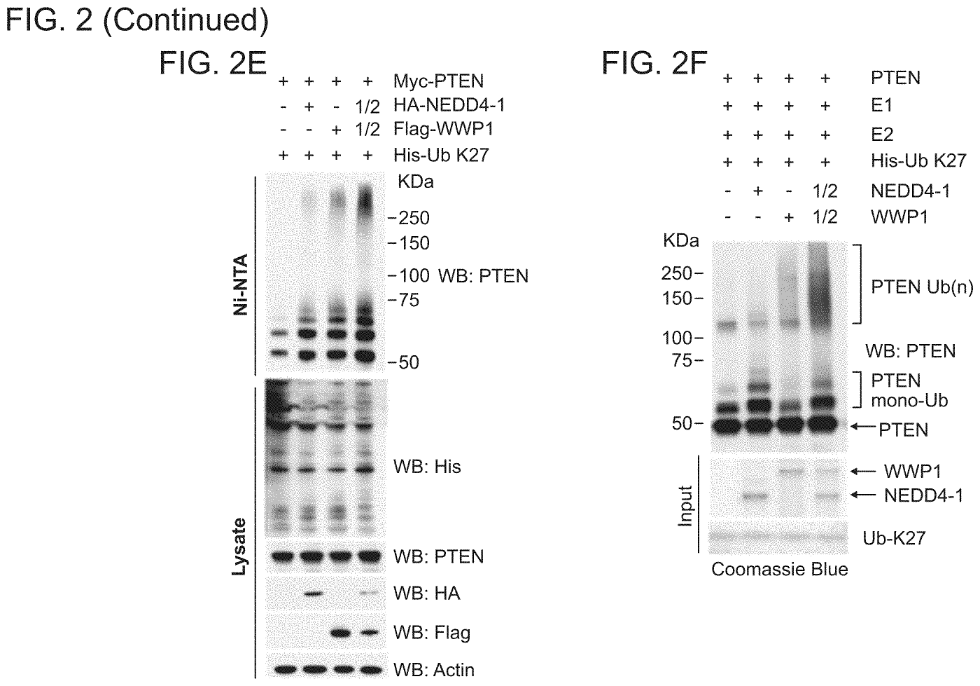

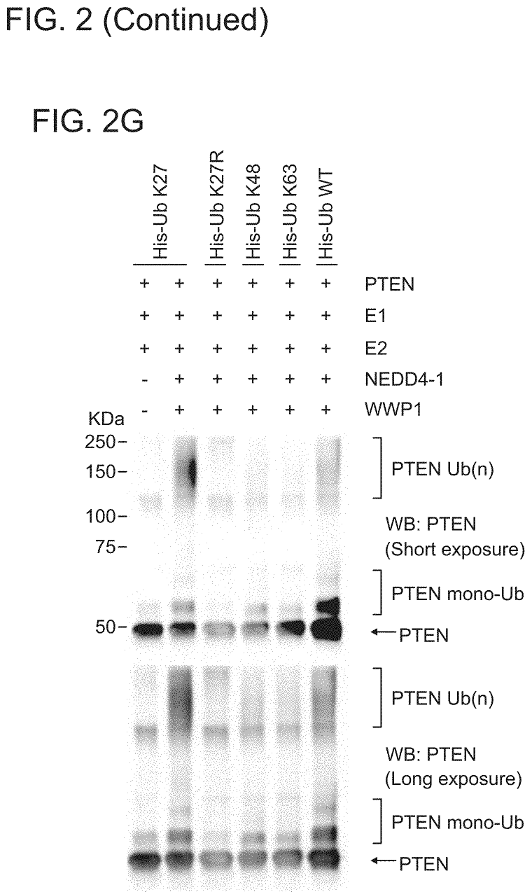

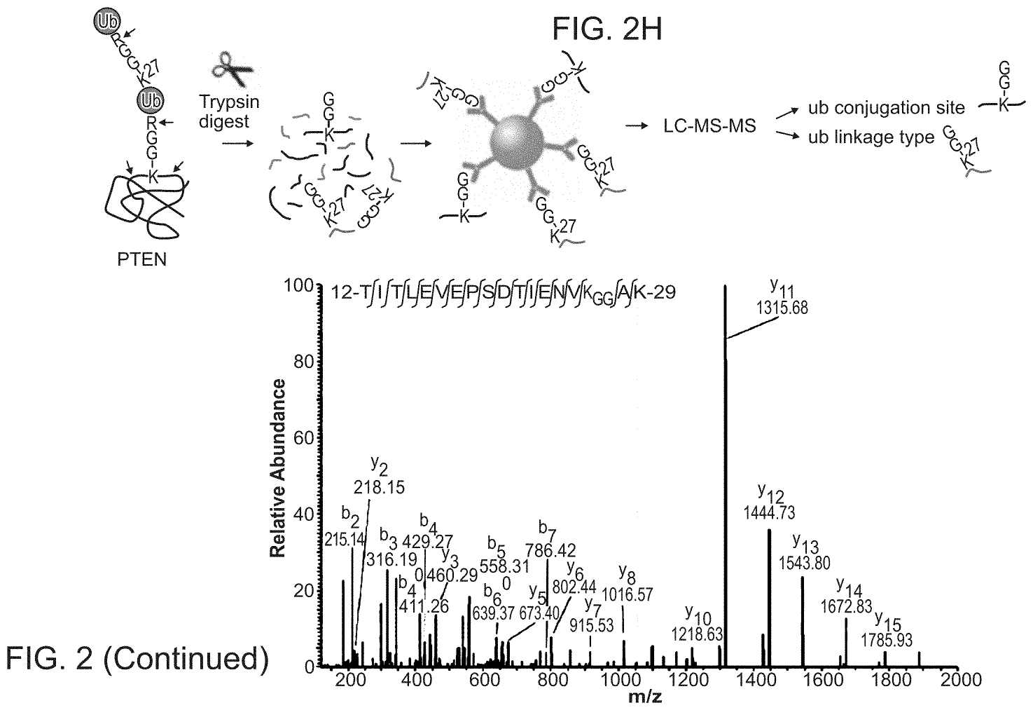

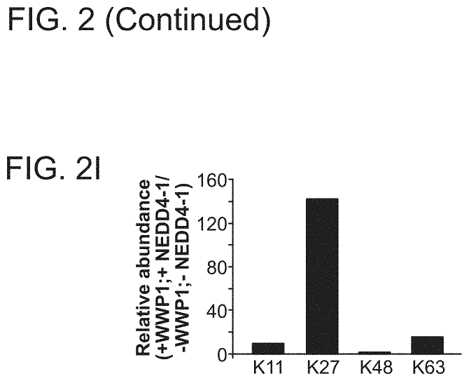

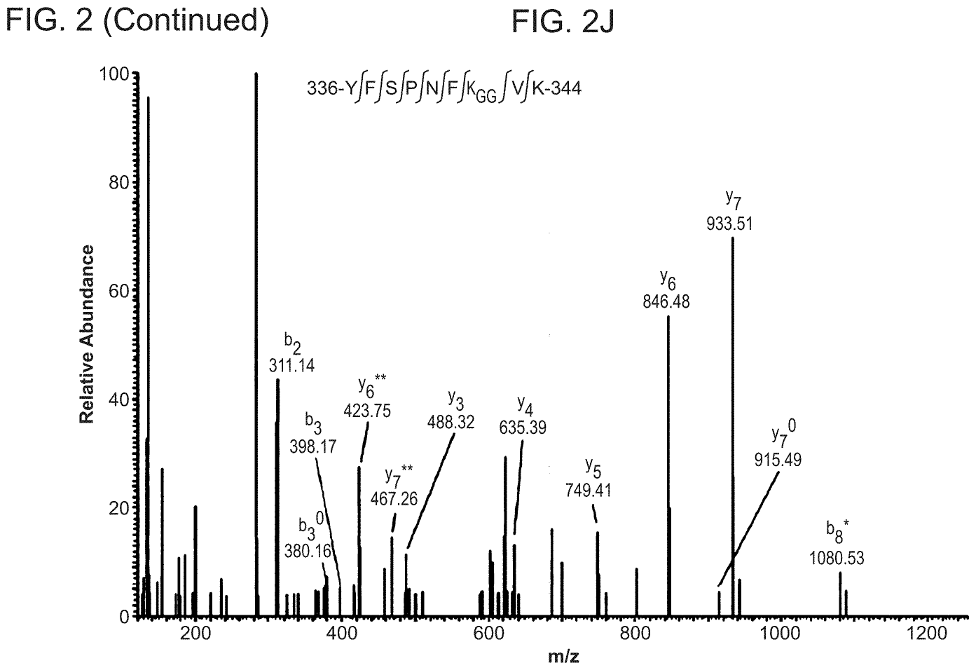

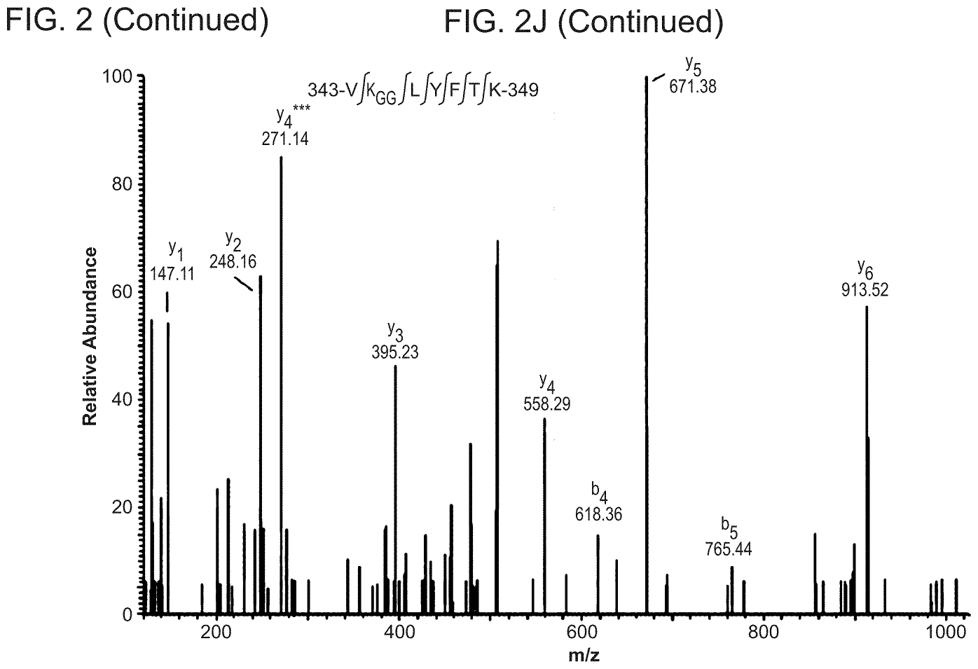

In order to identify the E3 ligases that induce PTEN atypical K27-linked poly-ubiquitination, exogenous PTEN was immunoprecipitated from DU145 cells, followed by identification of the proteins that interacted with PTEN by mass spectrometry (FIG. 2A; FIG. 18). Two NEDD4-1 family HECT-type ubiquitin E3 ligases, WWP1 and NEDD4-1, were shown to interact with PTEN, and that WWP1 exhibited the strongest binding capability among the NEDD4-1 family ubiquitin E3 ligases; indicating the role of WWP1 as a substrate adaptor protein (FIG. 1B). Intriguingly, it was also found that these two ubiquitin E3 ligases could interact with each other both in vivo and in vitro (FIG. 2B, FIG. 2C).

To further analyze which kind of ubiquitin chain type was generated on PTEN by the WWP1/NEDD4-1 E3s, the aforementioned ubiquitin mutants were used. Strikingly, only the K27R ubiquitin could block WWP1/NEDD4-1 mediated PTEN poly-ubiquitination (FIG. 2D). Next a K27-only ubiquitin was used, which retains only the K27 lysine residue with the other six lysine residues were mutated to arginine, in order to corroborate that WWP1/NEDD4-1 complex is a bona-fide E3 towards PTEN K27-linked poly-ubiquitination. These ubiquitination assays revealed that overexpression of either WWP1 or NEDD4-1, but not other NEDD4 family E3 ligases, robustly stimulated K27-linked PTEN poly-ubiquitination (FIG. 1C). Moreover, the overexpression of WWP1 along with NEDD4-1 showed synergistic effects on PTEN K27-linked poly-ubiquitination, as compared to NEDD4 or WWP1 alone (FIG. 2E).

To determine the possible direct role of the WWP1/NEDD4-1 E3s in PTEN poly-ubiquitination, an in vitro ubiquitination assay was performed. It was found that incubation of purified NEDD4-1 alone could not efficiently promote PTEN K27-linked poly-ubiquitination, whereas incubation of WWP1 alone only slightly triggered PTEN poly-ubiquitination. However, in the presence of both WWP1 and NEDD4-1, PTEN was robustly K27-linked poly-ubiquitinated (FIG. 2F). To further corroborate that the WWP1/NEDD4-1 E3s specifically trigger PTEN K27-linked poly-ubiquitination, various ubiquitin mutants were utilized. Notably, while K48- and K63-ubiquitin could not significantly trigger WWP1/NEDD4-1-mediated PTEN poly-ubiquitination, the K27-ubiquitin promoted chain formation on PTEN as significantly as the wild-type ubiquitin (FIG. 2G). Importantly, although the WWP1/NEDD4-1 E3s showed remarkable effects on PTEN K27-linked poly-ubiquitination, the protein turnover rate of PTEN was not affected when WWP1 or/and NEDD4-1 were overexpressed (FIG. 1D).

### Example 2—Identification of the Lysine Residues Responsible for PTEN K27-Linked Poly-Ubiquitination

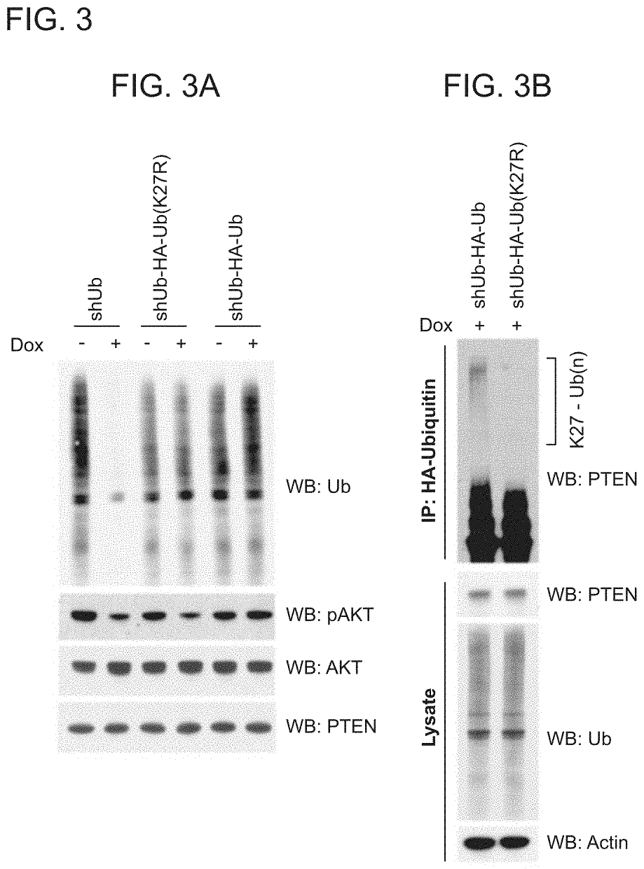

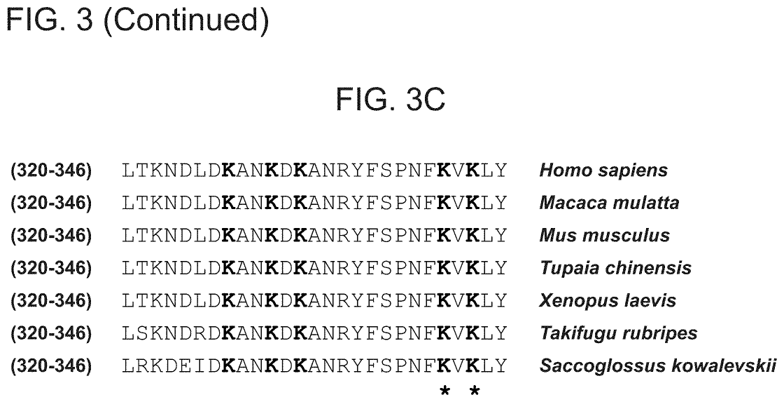

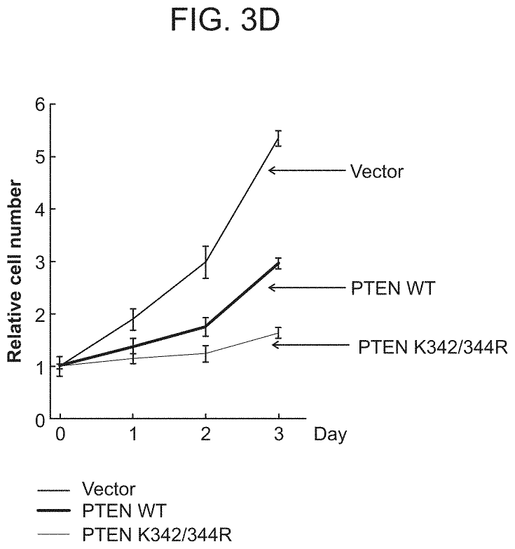

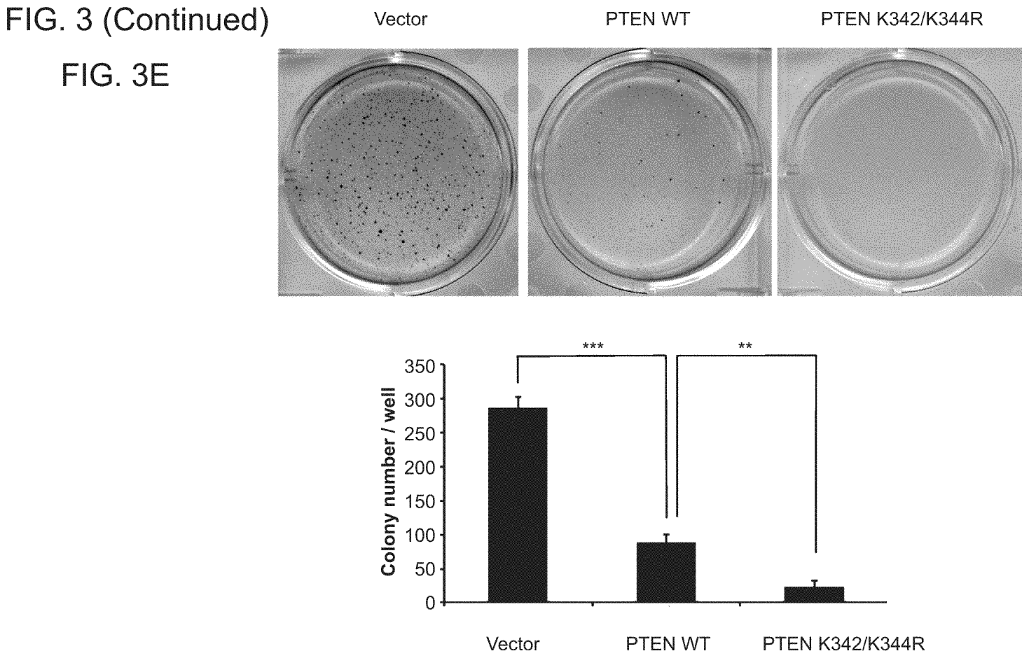

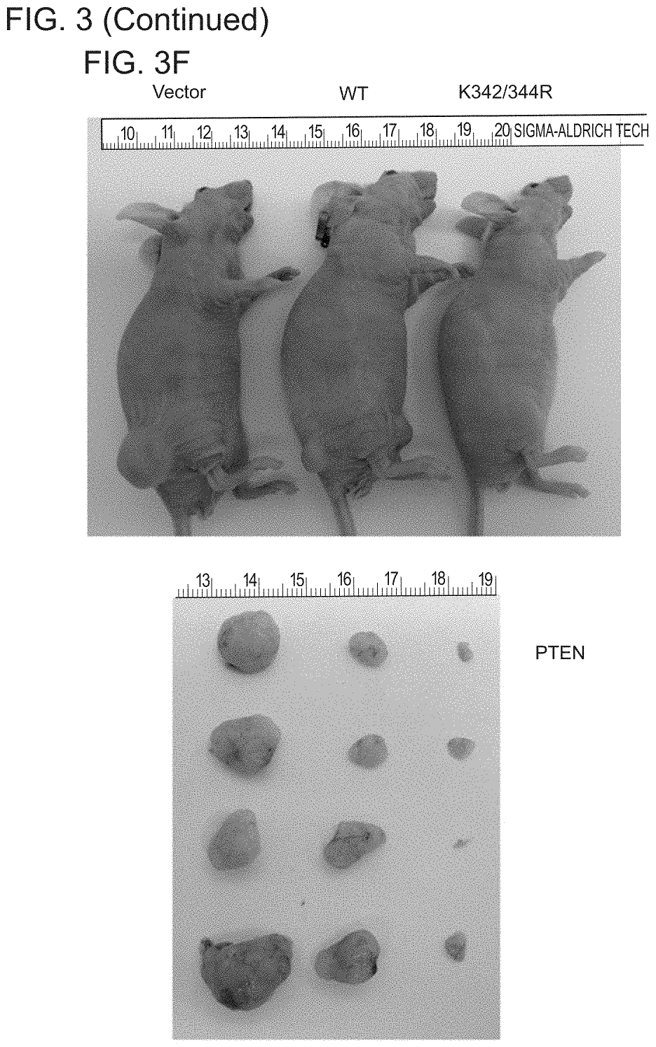

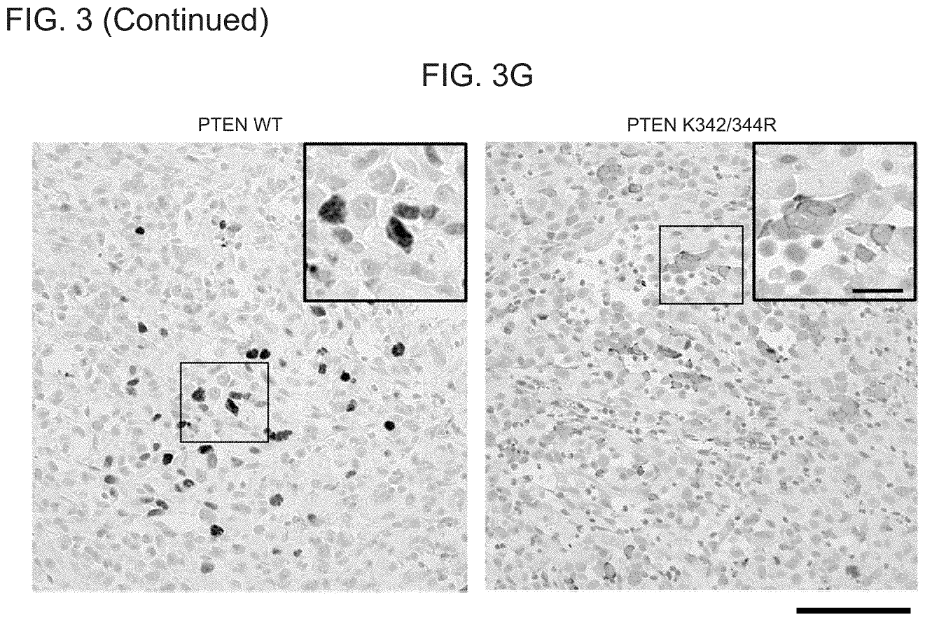

To identify the PTEN ubiquitination conjugation sites, mass spectrometry (MS) analysis was performed on ubiquitinated PTEN peptides isolated from control or WWP1/NEDD4-1 overexpressing cells by PTEN immunoprecipitation, digestion with trypsin and subsequent enrichment with an anti-K-ε-GG antibody. Strikingly, this analysis revealed that an ubiquitin peptide with a di-Gly modification on the K27 residue was markedly increased upon overexpression of WWP1/NEDD4-1 E3 complex, whereas ubiquitin modification on other Lys residues, such as K11, K48 and K63, was only modestly increased (FIG. 2L, FIG. 2H, FIG. 2I). Moreover, MS analysis identified PTEN K342 and K344 residues as being modified by ubiquitin in WWP1/NEDD4-1 overexpression cells (FIG. 2J). In keeping with the MS analysis, mutation of K342 and K344 residues to Arginine (R) substantially reduced WWP1/NEDD4-1 mediated PTEN K27-linked poly-ubiquitination (FIG. 2K), while the protein turn over rate of PTEN K342/K344R mutant was similar to that of wild type (WT) PTEN (FIG. 1E). This analysis therefore provides multiple lines of evidence for the formation of a K27-linked poly-ubiquitin chain on PTEN K342 and K344 residues mediated by the WWP1/NEDD4-1 E3 complex. Further, this atypical poly-ubiquitination would not interfere with the stability of the PTEN protein, consistent with previous reports showing that K27-linked poly-ubiquitination is not a signal for protein degradation. Importantly, this K342/K344 lysine patch is well conserved across species, as shown in FIG. 3A, FIG. 3B, and FIG. 3C. Moreover, the catalytic domain of either WWP1 or NEDD4-1 is also highly conserved throughout evolution, suggesting that the WWP1/NEDD4-1 mediated K27-linked poly-ubiquitination is evolutionarily conserved and important in the regulation of the PTEN function across species.

### Example 3—the Essential Role of WWP1/NEDD4-1 E3s in K27-Linked PTEN Poly-Ubiquitination

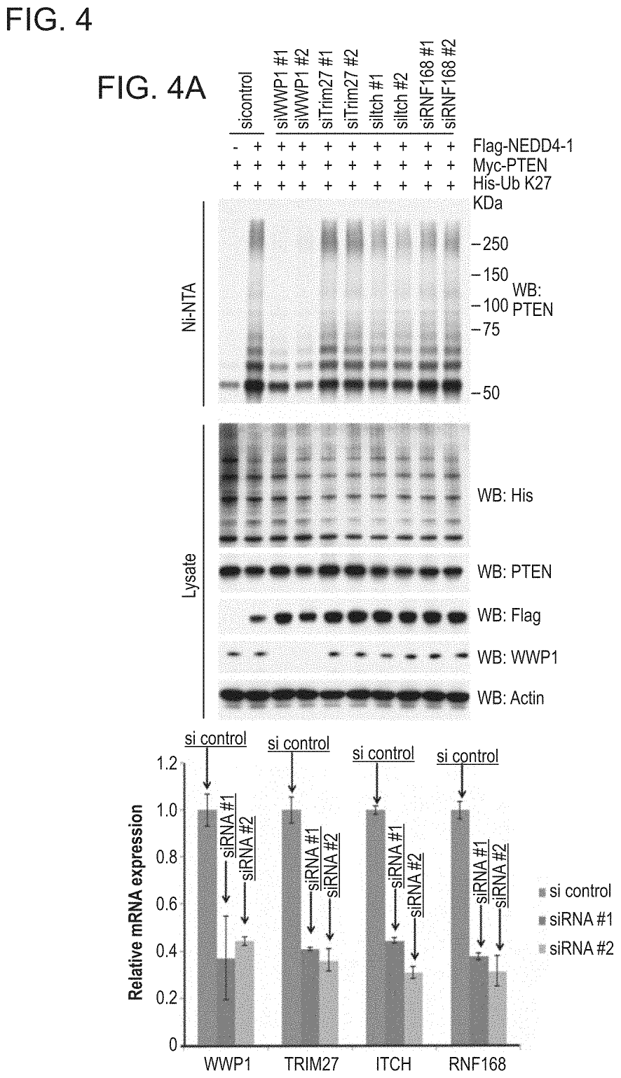

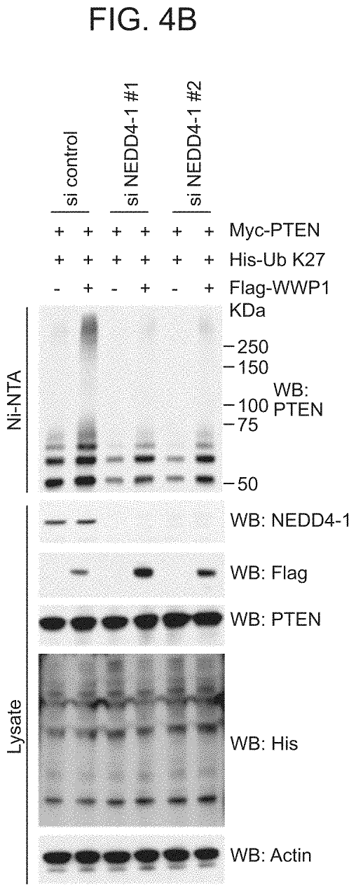

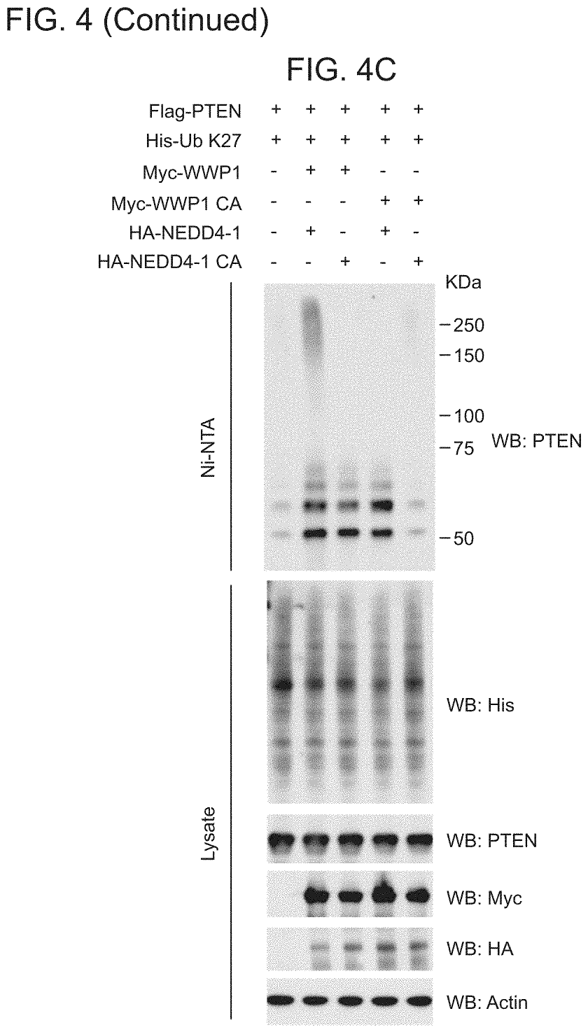

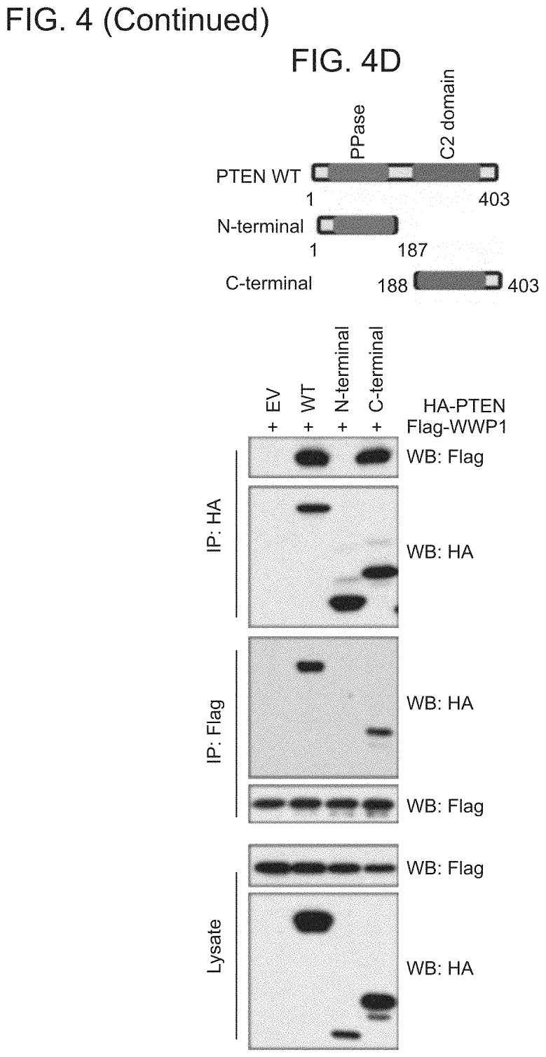

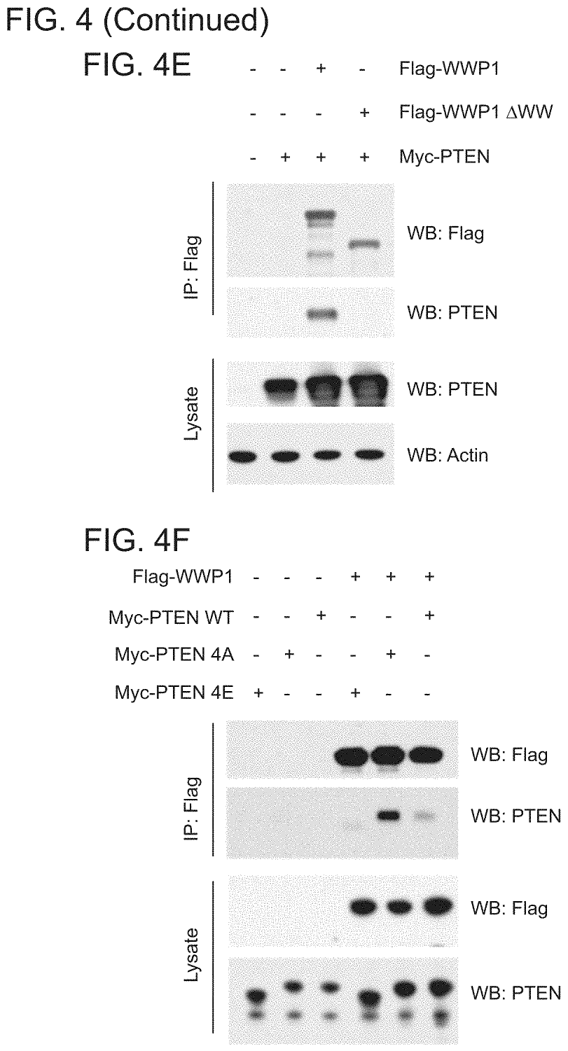

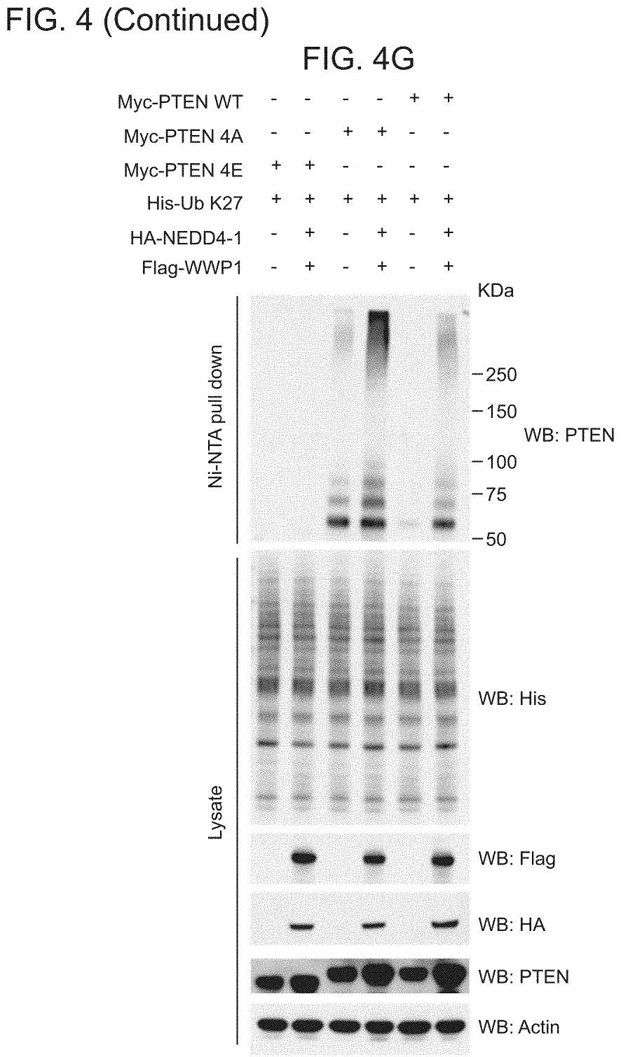

To further define the respective role of WWP1 and NEDD4-1 in K27-linked PTEN poly-ubiquitination, the effect of knockdown either WWP1 or NEDD4-1 on PTEN poly-ubiquitination was examined. Notably, depletion of WWP1 markedly reduced NEDD4-1 mediated PTEN K27-linked poly-ubiquitination, whereas siRNAs, which targets the other E3 ligases, showed little effects on PTEN poly-ubiquitination (FIG. 4A). Similarly, depletion of NEDD4-1 via siRNAs drastically impaired WWP1-mediated PTEN K27-linked poly-ubiquitination, indicating that either WWP1 or NEDD4-1 is indispensable to promote K27-linked PTEN poly-ubiquitination (FIG. 4A, B). Next, whether catalytic activity of either WWP1 or NEDD4-1 is required for PTEN K27-linked poly-ubiquitination was examined. It was found that overexpression of either a catalytic defective WWP1 (C890A) or NEDD4-1 mutant (C867A) robustly abrogated PTEN K27-linked poly-ubiquitination. Synergistically, overexpression of both WWP1 and NEDD4-1 catalytic mutants not only significantly reduced PTEN poly-ubiquitination, but also mono-ubiquitination, indicating that the catalytic activity of either WWP1 or NEDD4-1 is required for PTEN poly-ubiquitination (FIG. 4C).

WWP1 could act as a PTEN substrate adaptor (FIG. 1B). Therefore, the domains that are required for the interaction between WWP1 and PTEN protein were mapped. Various PTEN functional domains were co-expressed along with full-length Flag-WWP1. The reciprocal immunoprecipitation assays indicated that WWP1 interacts with both the full-length (1-403) and C-terminal (188-403), but not the N-terminal PTEN (1-187) (FIG. 4D). Reciprocally, it was found that WW domain of WWP1 is required for its interaction with PTEN (FIG. 4E), consistent with the findings demonstrating that the WW domain is the substrate interaction motif among HECT type E3 ligases.

It is known that PTEN activity is strictly controlled by C-terminal phosphorylation (S380, T382, T383, and S385). C-terminal phosphorylation favors a PTEN inactive “close” conformational state. The phosphorylation defective 4A mutant of PTEN is active, “open” and localized at plasma membrane, whereas phosphomimetic 4E is cytosolic and inactive. Which PTEN state would be an effective substrate for WWP1/NEDD4-1 E3s was determined. It was found that WWP1 preferentially interacted with PTEN 4A, but not the PTEN 4E mutant (FIG. 4F). Consistently, an in vivo ubiquitination assay revealed that the WWP1/NEDD4-1 E3s specifically triggered PTEN 4A K27-linked poly-ubiquitination, but not PTEN 4E mutant, suggesting that WWP1/NEDD4-1 E3 preferentially acted on the active “open” pool of PTEN (FIG. 4G).

### Example 4—K27-Linked PTEN Poly-Ubiquitination Suppresses PTEN Dimerization, Membrane Recruitment and Function

Ubiquitination can impose a spatial hindrance for protein-protein interaction. Thus, whether WWP1/NEDD4-1 mediated K27-linked PTEN ubiquitination regulates the PTEN dimer formation was examined. To this end, PTEN dimerization was monitored in vitro by purification of Flag-tagged un-modified or ubiquitinated PTEN on beads, followed by testing its interaction with GST-PTEN from bacteria. Intriguingly, it was found that overexpression of WT ubiquitin along with the WWP1/NEDD4-1 E3, which resulted in K27-linked poly-ubiquitination of PTEN, impaired the interaction of Flag-PTEN with GST-PTEN.

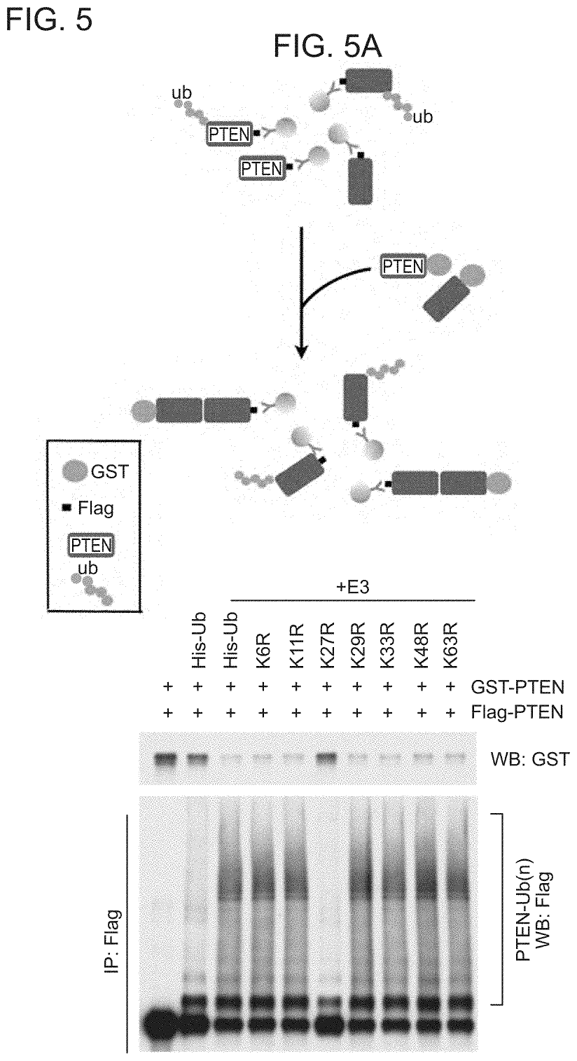

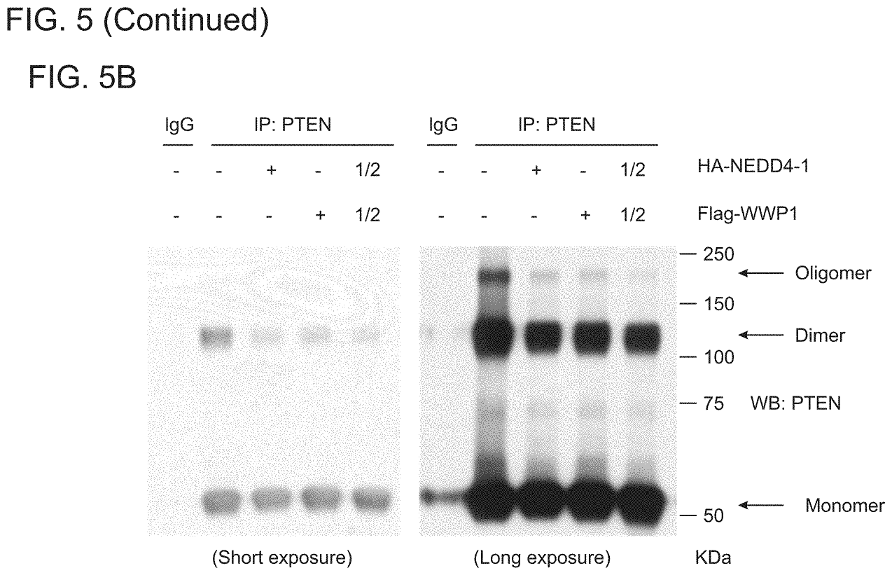

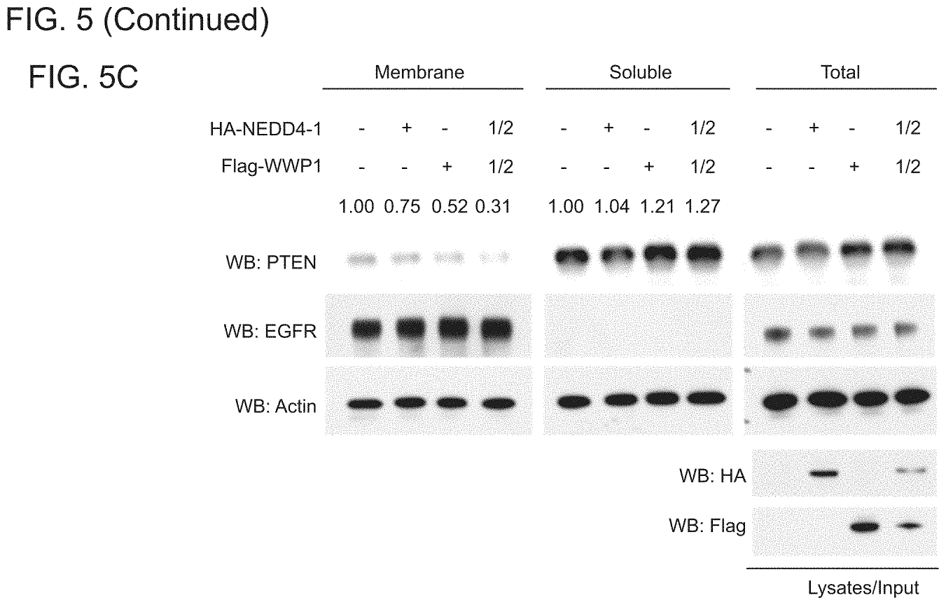

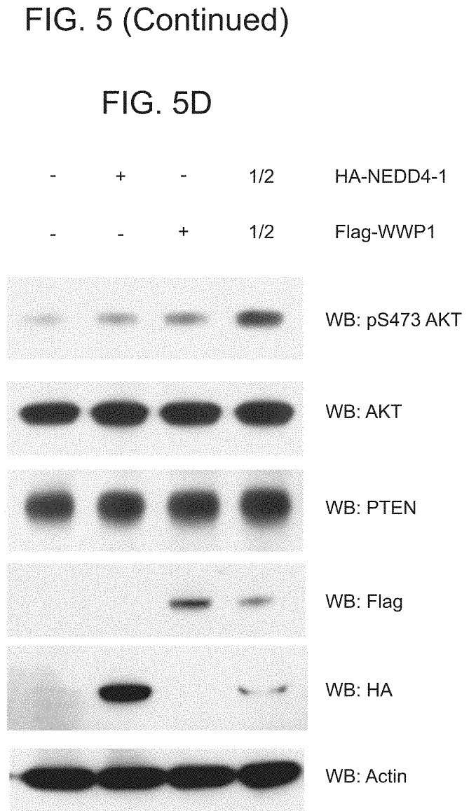

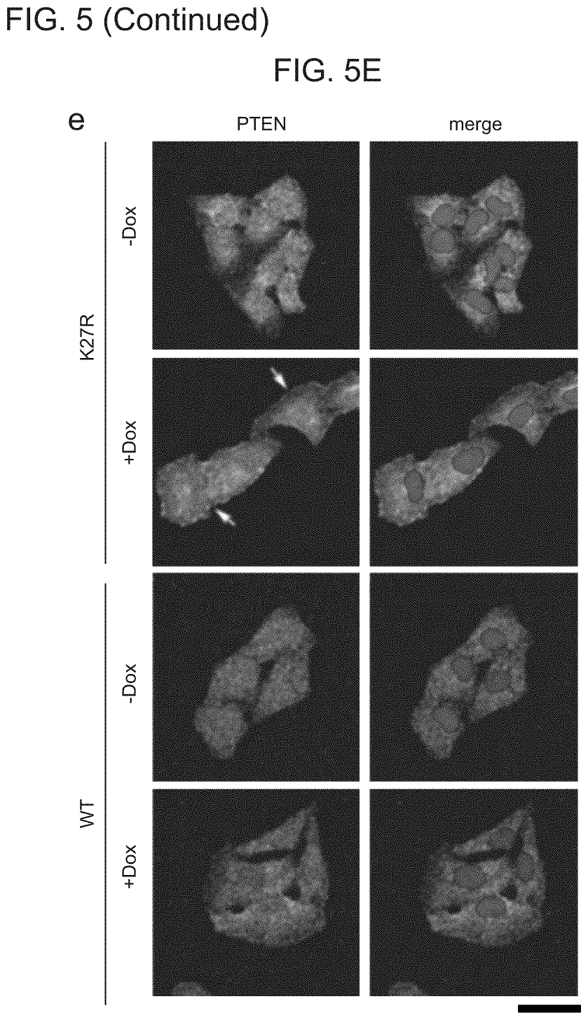

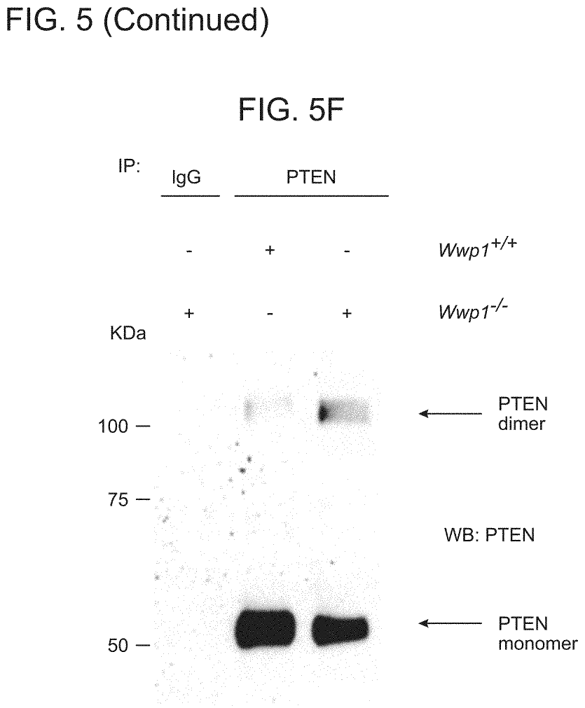

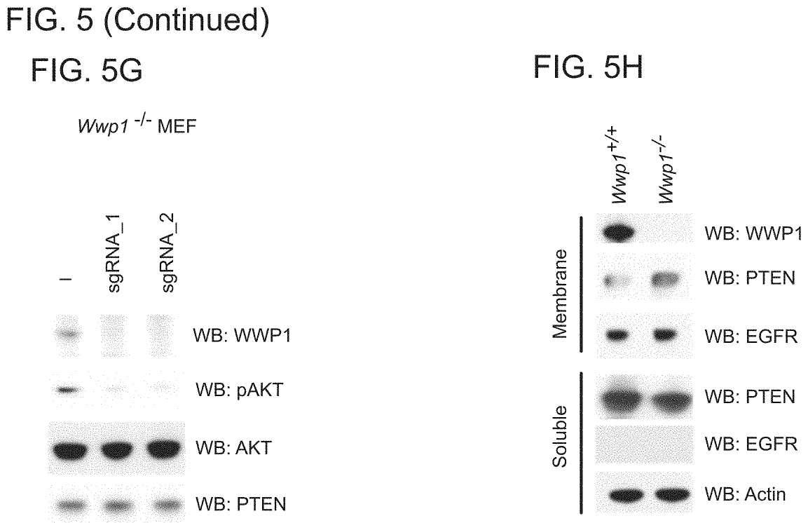

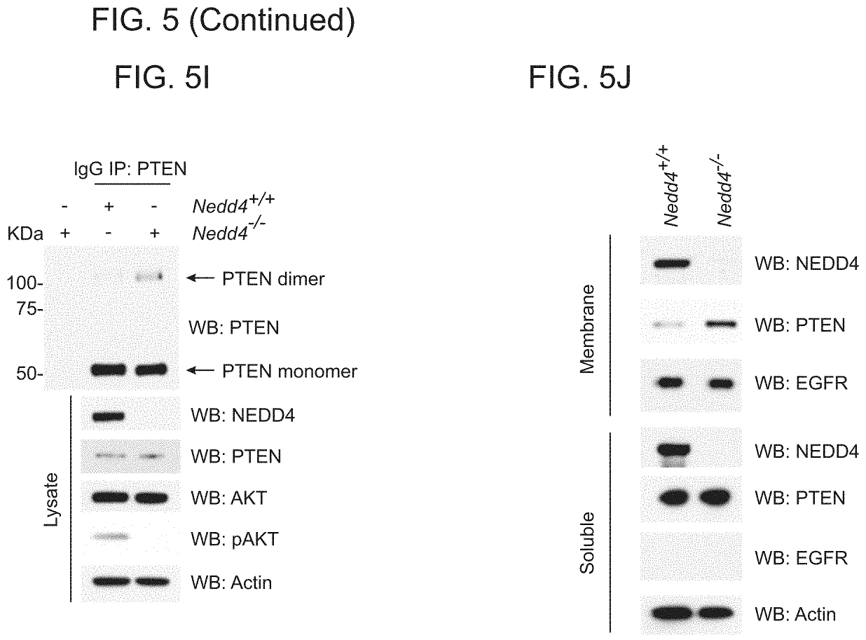

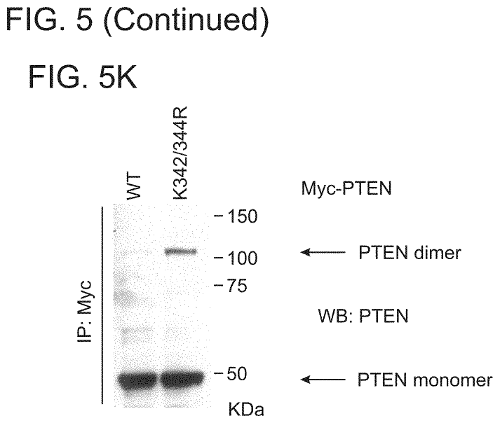

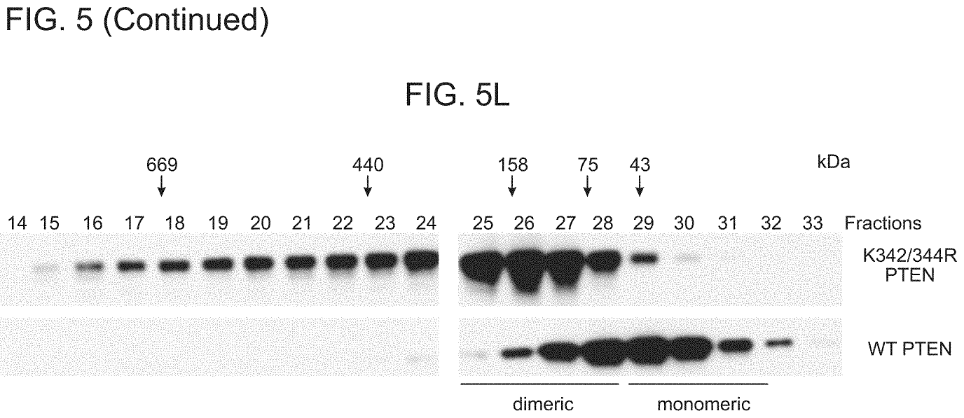

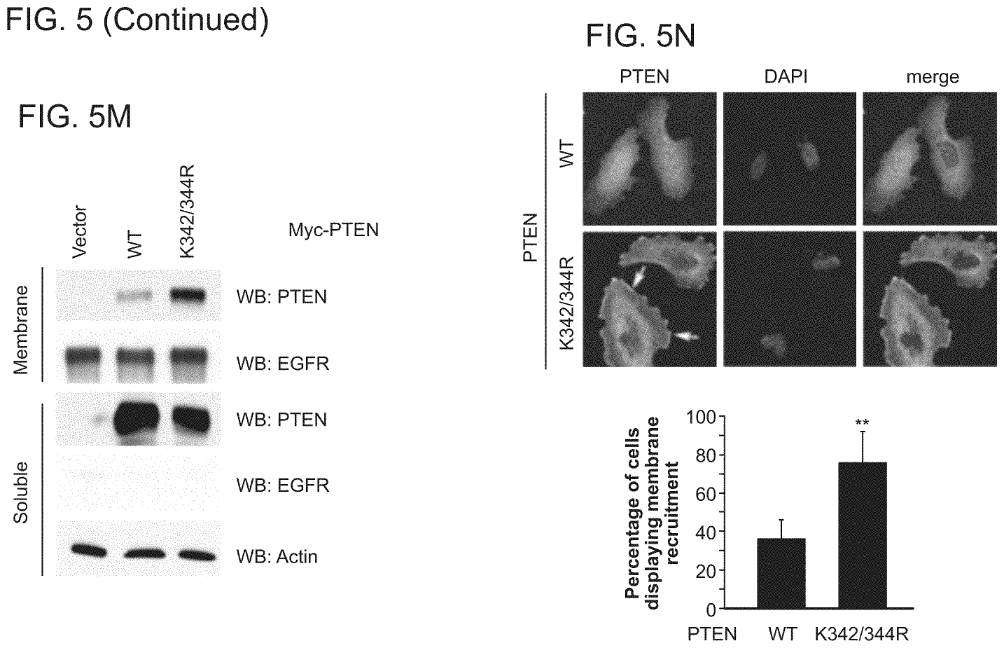

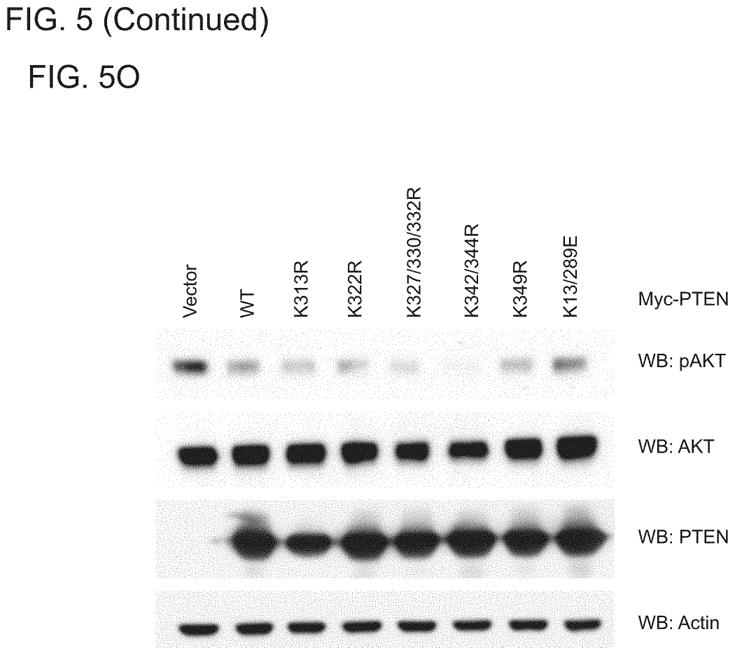

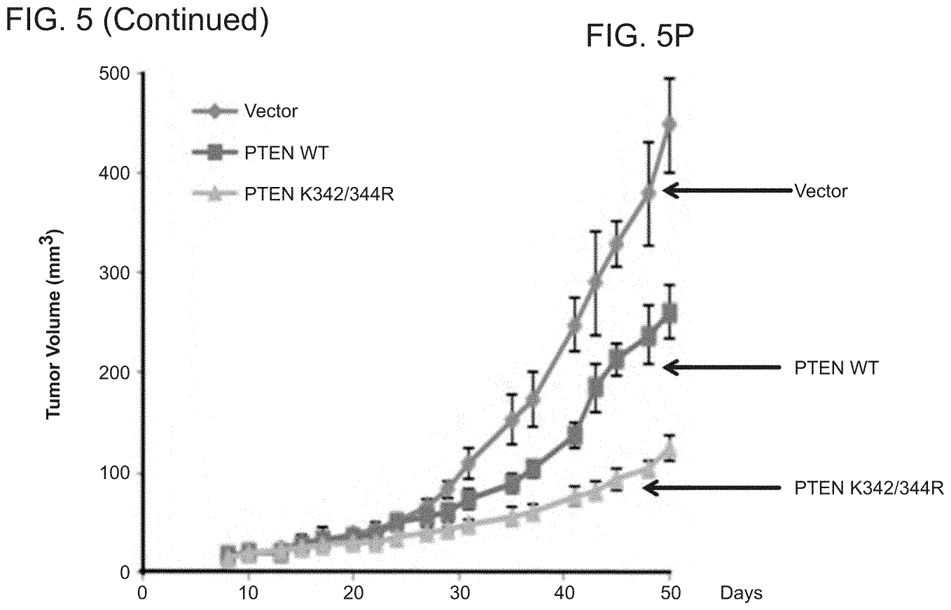

By contrast, overexpression of the K27R mutant ubiquitin together with the WWP1/NEDD4-1 E3 complex did not affect the interaction of Flag-PTEN with GST-PTEN, suggesting that K27-linked poly-ubiquitination of PTEN inhibits its dimerization (FIG. 5A). Consistent with this notion, non-reducing/non-denaturing gel assays showed that WWP1 and NEDD4-1 synergistically suppressed PTEN dimer/oligomerization (FIG. 5B).

Given that PTEN dimerization is associated with PTEN membrane recruitment and activation, the effect of WWP1/NEDD4-1 mediated K27-linked PTEN poly-ubiquitination on PTEN membrane recruitment and its activity was investigated. Fractionation experiments showed that overexpression of WWP1 together with NEDD4-1 synergistically suppressed PTEN membrane recruitment (FIG. 5C), in turn resulting in synergistic AKT activation (FIG. 5D).

Next, a ubiquitin replacement system was utilized that allows simultaneous depletion of endogenous ubiquitin and expression of exogenous ubiquitin, or its variants, by doxycycline. It was found that replacement of endogenous ubiquitin with a K27R mutant, but not wild-type ubiquitin, not only inhibited PTEN poly-ubiquitination, but also induced PTEN localized to the plasma membrane and, in turn, suppressed AKT activation (FIG. 5E; FIG. 7A, FIG. 7B).

To further corroborate the role of WWP1 and NEDD4-1 in PTEN dimerization, membrane recruitment and function, Wwp1^{-/-} MEFs were generated using a CRISPR/Cas9 gene editing approach (see Methods). Genetic ablation of WWP1 resulted in increased PTEN dimerization and membrane recruitment, as evaluated by non-reducing/non-denaturing gel and membrane fractionation analyses, which in turn suppressed AKT activity (FIG. 5F, FIG. 5G, FIG. 5H). Similar results were also obtained in Nedd4^{-/-} MEFs (FIG. 5I, FIG. 5J). Collectively, these data support a new role for WWP1/NEDD4-1 mediated K27-linked poly-ubiquitination on the suppression of PTEN dimerization, membrane recruitment and function.

Consistent with the critical role of K342/K344 residues on PTEN in WWP1/NEDD4-1 mediated K27-linked poly-ubiquitination (FIG. 5L, FIG. 5J, FIG. 5K), it was found that the PTEN K342/K344R ubiquitination defective mutant displayed significant dimerization potential (FIG. 5K). Moreover, gel filtration assays revealed that the PTEN K342/K344R mutant formed more oligomeric/dimeric complexes, whereas PTEN WT predominately formed monomeric complex in vivo, thus further highlighting the suppressive roles of K27-linked poly-ubiquitination in PTEN oligomerization/dimerization (FIG. 5L). Accordingly, the PTEN K342/K344R mutant showed a marked plasma membrane accumulation, as detected by membrane fractionation and confocal analyses (FIG. 5M, FIG. 5N, FIG. 5P). Functionally, the PTEN K342/K344R mutant exhibited the strongest inhibition on AKT activity, as compared to other PTEN mutants and wild-type control (FIG. 5O).

Finally, to determine the influence of PTEN K27-linked poly-ubiquitination on cell proliferation, tumorigenic potential and tumour growth in vivo, the tumour suppressive function of WT and ubiquitination-defective K342/K344R PTEN mutant were compared. Remarkably, in comparison to WT PTEN, the PTEN K342/K344R mutant induced a stronger inhibition of cell proliferation and anchorage-independent growth (FIG. 3D, FIG. 3E). Next, the influence of NEDD4-WWP1/PTEN crosstalk on tumour growth in vivo was evaluated by employing a xenograft tumour model. Notably, tumors derived from PTEN K342/344R mutant transduced cells grew at a slower rate than those derived from WT PTEN, thus indicating that the PTEN K342/K344R mutant exhibits stronger inhibition of tumorigenesis than WT PTEN (FIG. 5M and representative images shown in FIG. 3F. Consistently, immunohistochemistry (IHC) analyses of tumors derived from PTEN WT or K342/K344R mutants revealed that PTEN K342/K344R displayed more plasma membrane accumulation phenotypes, whereas PTEN WT was dispersed within the cells (FIG. 3G). Together, the data suggest that WWP1/NEDD4-1 mediated K27-linked PTEN poly-ubiquitination on PTEN K342/K344 residues inhibits PTEN tumour suppressor function by dissociation of PTEN from plasma membrane compartment both in vitro and in vivo.

### Example 5—MYC Transactivates WWP1/NEDD4-1 Gene Expression Towards PTEN Suppression

Because activation of the PI3K-AKT signaling pathway and MYC amplification frequently occur together in cancers and correlate with a high histological grade and poor prognosis, the ability of MYC to inhibit PTEN function, thereby activating PI3K-AKT pathways was tested. As MYC functions as a transcription factor, whether WWP1 and NEDD4-1 were putative MYC target genes was tested. Through analyses of Transcription factor Affinity Prediction (TRAP) software (http://trap.molgen.mpg.de/cgi-bin/home.cgi) and Chromatin Immunoprecipitation (ChIP)-seq databases (https://genome.ucsc.edu/ENCODE/), putative MYC responsive elements in the promoter of both Wwp1 and Nedd4-1 genes were identified (FIG. 6A). ChIP assays confirmed that MYC protein was bound to the predicted regions on the promoter of both WWP1 and NEDD4-1 (FIG. 6A). Consistent with this observation, overexpression of MYC dose-dependently increased the level of WWP1 and NEDD4-1 at both mRNA and protein level, suggesting that both WWP-1 and NEDD4-1 are MYC target genes (FIG. 6B, FIG. 6C). As a result of WWP1/NEDD4-1 upregulation, AKT activation was significantly increased upon MYC overexpression, in the absence of any change in PTEN protein levels (FIG. 6C).

Conversely, depletion of MYC by siRNA SMARTpools led to the suppression of both WWP1 and NEDD4-1 protein levels and a concomitant decrease in AKT activation, again without any change in PTEN protein levels, further corroborating that MYC is an upstream regulator of the WWP1/NEDD4-1 pathway (FIG. 6D).

Given that MYC markedly induces WWP1 and NEDD4-1 expression, whether MYC could trigger PTEN K27-linked poly-ubiquitination to suppress PTEN dimerization, membrane recruitment and function was examined. Indeed, overexpression of MYC not only promoted PTEN K27-linked poly-ubiquitination, but also disrupted PTEN dimerization, and its association with the plasma membrane compartment in a dose dependent manner, as assessed by in vivo ubiquitination, immunoprecipitation and membrane fractionation analyses, (FIG. 6E, FIG. 6, FIG. 6G).

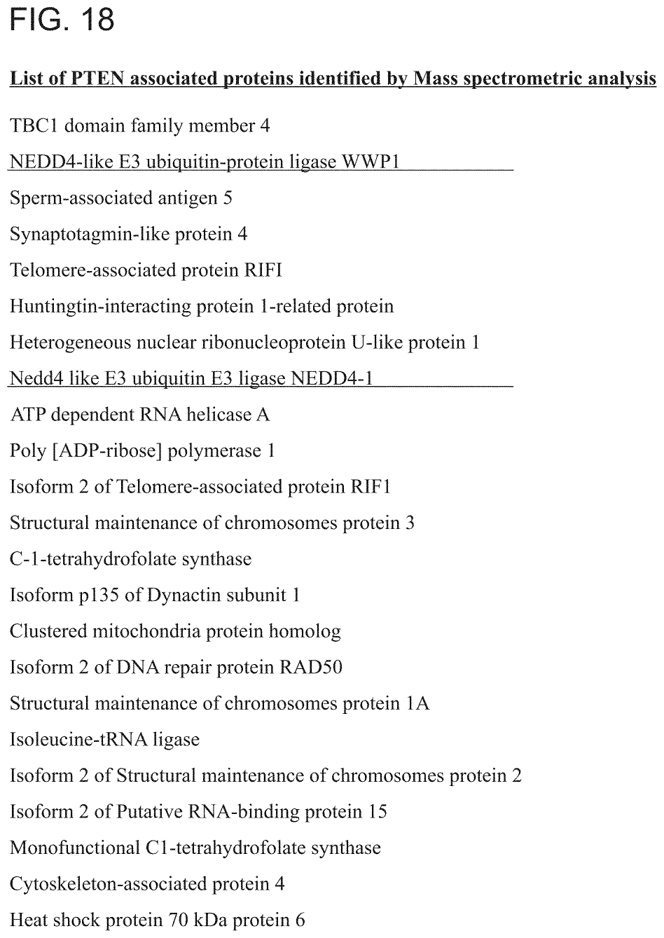

To address the role of WWP1 and NEDD4-1 downstream of MYC activation, shRNAs against either WWP1 or NEDD4-1 to knockdown both WWP1 and NEDD4-1 were utilized, and the PTEN ubiquitination status and function with or without MYC stable overexpression in DU145 cells was examined. Remarkably, while depletion of both WWP1 and NEDD4-1 decreased PTEN K27-linked poly-ubiquitination in control cells, this effect was much more prominent in MYC overexpression cells (FIG. 6E). Functionally, depletion of either WWP1 or NEDD4-1, or both, triggered a strong inhibition of MYC-induced AKT activation without affecting PTEN protein level; supporting the critical roles of WWP1 and NEDD4-1 in MYC-dependent AKT activation (FIG. 6H, FIG. 61).

Next, to determine the impact of NEDD4-1 and WWP1 on MYC-induced tumorigenic potential, the aforementioned MYC stable DU145 cells were utilized (FIGS. 6E and H). Depletion of both WWP1 and NEDD4-1 led to a reduction in the formation of soft-agar colonies, whereas this inhibitory effect on the formation of colonies was much more prominent in MYC overexpressing cells (FIG. 6J). To investigate whether the reduction of the soft-agar colonies caused by WWP1/NEDD4-1 knockdown is due to apoptosis or their influence on the cell-cycle, cells were stained with Propidium Iodide (PI), followed by FACS analysis. It was found that depletion of WWP1/NEDD4-1 has little effect on cell-cycle profile (FIG. 7A), but does lead to a more prominent apoptosis in MYC overexpression cells (FIG. 6K). Collectively, our data demonstrates the critical roles of WWP1/NEDD4-1 in MYC-induced tumour progression through PTEN functional inhibition, in turn suggesting that targeting WWP1/NEDD4-1 could provide a therapeutic strategy to treat MYC-driven cancers.

### Example 6—Aberrant WWP1/NEDD4-1/PTEN Crosstalk in Human Cancer

The patho-physiological relevance of the WWP1/NEDD4-1/PTEN crosstalk in human cancer was investigated. First, the various human prostate cancer (CaP) cell lines were examined. Strikingly, the expression of WWP1 and NEDD4-1 was inversely correlated with PTEN accumulation in membrane fraction, whereas PTEN displayed no significant correlation with NEDD4 and WWP1 expression in total and soluble fractions (FIG. 7B). To further validate the clinical relevance of this functional crosstalk in human cancer, the subcellular localization of PTEN and NEDD4-1/WWP1 expression in consecutive slides from 126 human CaP patients was examined. Immunohistochemistry (IHC) analysis revealed significant up-regulation of NEDD4-1 and concomitant loss of PTEN membrane recruitment in human CaP specimens (FIG. 6L and FIG. 6M, and representative images shown in FIG. 7C). Moreover, NEDD4-1 expression was positively correlated with tumor grade, whereas a striking negative correlation was observed with PTEN membrane recruitment (FIG. 6M). Furthermore, the number of patients with a signature of high NEDD4 expression and loss of PTEN membrane recruitment increased with the tumour grade, and a significant difference was observed between Gleason score of 6/7 and Gleason score of 8/9 (FIG. 6N).

Through bioinformatics analysis, it was found that the level of WWP1 and NEDD4-1 transcripts was positively correlated in human CaP samples (TCGA Provisional and MSKCC, Cancer Cell 2010) (FIG. 6O). Importantly, the level of MYC transcripts was positively correlated with both the level of WWP1 and NEDD4-1 transcripts (TCGA Provisional) (FIG. 7D). Intriguingly, WWP1, which is localized on chromosome 8q21, one of the regions frequently amplified in many human cancers such as breast and prostate cancer and a region where MYC is also localized, was found co-amplified with MYC in human CaP specimens (FIG. 6A, FIG. 6B, FIG. 6C; and FIG. 8). Collectively, these findings lend support to the relevance of aberrant MYC/WWP1/NEDD4-1/PTEN crosstalk in human tumorigenesis and in CaP progression.

### Example 7—Inactivation of WWP1 in the Mouse Suppresses MYC-Driven Prostate Tumorigenesis

To gain in vivo genetic evidence that WWP1 loss would inhibit MYC-driven prostate tumors, the experiments of this example took advantage of Hi-Myc mice, which express Hi-Myc in a prostate epithelium-specific manner that result in complete penetrance of high-grade prostatic intraepithelial neoplasia (PIN) at 3 months age and progress into invasive adenocarcinoma within 5 to 12 months of age (Ellwood-Yen et al., 2003). The Hi-Myc mice were crossed the with Wwp1 heterozygous mice to obtain cohorts of Hi-Myc; Wwp1^{+/-} mutants or Hi-Myc; Wwp1^{+/+} controls. Consistently, it was found that heterozygous Wwp1 deletion significantly suppressed Hi-Myc driven tumorigenesis (FIG. 9A). Histological analyses corroborated that prostates from Wwp1 heterozygous mice displayed significant reduction of Hi-Myc driven high-grade PIN, as compared to Wwp1 WT counterparts (FIG. 9B). In keeping with this notion, heterozygous Wwp1 deletion also inhibited AKT activation without affecting PTEN protein level in prostate derived lysates (FIG. 9C). Lastly, IHC analyses of PTEN protein in DLP tissue revealed that Wwp1 heterozygous mice showed intense PTEN plasma membrane accumulation in prostate epithelial cells compared to Wwp1 WT counterparts (FIG. 9D). Thus, heterozygous genetic ablation of Wwp1 triggers PTEN reactivation to prevent and suppress Myc-driven tumorigenesis in vivo.

### Example 8—Identification of indole-3-carbinol (I3C) as a WWP1 inhibitor

I3C is a natural compound and is produced by the breakdown of the glucosinolate glucobrassicin, which can be found in relatively high levels in cruciferous vegetables, such as broccoli, cauliflower, cabbage, collard greens, brussel sprouts and kale, and displays negligible toxicity (Aggarwal and Ichikawa, 2005; Ahmad et al., 2010, 2012; Firestone and Sundar, 2009; Sarkar and Li, 2009). A previous study revealed that I3C could fit into the HECT domain of NEDD4-1 by in silico molecular modeling, hence interacting with NEDD4-1 with a calculated equilibrium dissociation constant of approximately 88.1 μM (Kd: ˜88.1 μM), thereby inhibiting NEDD4-1 activity (Aronchik et al., 2014). As the HECT catalytic domain of WWP1 is structurally similar to that of NEDD4-1, it was therefore hypothesized that I3C might also inhibit WWP1. A crystal structure of NEDD4-1 revealed the binding of a covalent inhibitor to its N-terminal lobe (Kathman et al., 2015). When I3C was superimposed onto the covalent inhibitor and WWP1 onto NEDD4-1, a model was generated showing I3C-bound WWP1 with the binding pocket formed by residues F577, L630, Y628, C629, N650, and Y656. The indole core of I3C could fit into the center of the N-terminal domain pocket and form extensive interactions with the surrounding hydrophobic residues (FIG. 10A), suggesting an inhibitory function of I3C towards the E3 ligase activity of WWP1. The experiments of this example next confirmed this hypothesis by MicroScale Thermophoresis (MST) binding analysis. Suprisingly, MST binding analysis revealed that I3C binds WWP1 at a dissociation constant of approximately 120 nM (Kd: ˜120 nM), with a significantly stronger binding affinity compared to NEDD4-1 (FIG. 10B, FIG. 10C, FIG. 10D).

### Example 9− Therapeutic Targeting of K27-Linked PTEN Poly-Ubiquitination Potently Suppresses Tumorigenesis In Vitro

Whether targeting WWP1/NEDD4-1, thereby enhancing PTEN dimerization and function, could offer a unique opportunity to treat MYC-driven cancers was investigated. As shown in FIG. 4A, FIG. 4B, and FIG. 4C, the activity of either WWP1 or NEDD4-1 is required for K27-linked PTEN poly-ubiquitination. Hence, using inhibitors that target either WWP1 or NEDD4-1 would be an ideal way to inactivate this E3 activity. As there is no small pharmacological inhibitor currently available to suppress/inhibit WWP1 activity, Indole-3-Carbinol (I3C), a potential WWP1 inhibitor, which suppresses the WWP1 activity through binding to its HECT domain, was used. First, to assess the on-target effects of I3C towards WWP1, Wwp1^{-/-} MEFs were used. Indeed, Wwp1^{-/-} cells were more resistant to I3C treatment, as compared to Wwp1^{+/+} cells (FIG. 10C).Next, whether inhibition of WWP1 by I3C treatment would affect PTEN K27-linked poly-ubiquitination and its subcellular localization in cells was tested. In line with the data above, inhibition of NEDD4-1 via I3C not only robustly diminished WWP1/NEDD4-1 E3 mediated PTEN K27-linked poly-ubiquitination, but also induced its plasma membrane accumulation, as evidenced by in vivo ubiquitination and confocal immunofluorescence analyses (FIG. 11A; FIG. 10D). Consequently, this led to a dose-dependent AKT suppression and a concomitant induction of apoptosis without affecting PTEN protein level in DU145 cells (PTEN competent), while only negligible effects were observed in PC3 cells (PTEN-null) (FIG. 11B). Similarly, I3C treatment showed drastic inhibition of cell growth in DU145 cells, but only modestly suppressed cell growth in both PC3 and LNCaP cells (PTEN-null); suggesting that I3C exerts its anti-proliferative effects at least in part in a PTEN-dependent manner (FIG. 11C). Additionally, two independent PTEN stably knocked-down cells were generated. Consistently, cells with PTEN depletion exhibited resistance to I3C treatment, as compared to control cells (FIG. 11D), confirming that I3C elicits its anti-proliferative effects through repression of PTEN K27-linked poly-ubiquitination.

The observation that WWP1/NEDD4-1 knockdown suppresses MYC-induced tumorigenic potential suggested the examination of whether deregulated MYC overexpression would show the “oncogenic addiction” of cells towards WWP1/NEDD4-1/PTEN axis to support cell growth.

To test this hypothesis, control cells were treated at a dose of I3C that had little effect on the cell growth. In marked contrast, cells became sensitized to this low dose of I3C treatment upon overexpression of MYC, suggesting that upon MYC overexpression, cells become dependent on the WWP1/NEDD4-1/PTEN axis for cell survival (FIG. 11E). The effect of I3C on primary prostate spheres derived from WT and prostate specific Myc transgenic mice (Hi-Myc) at 3 months of age was also examined. As expected, culture and serial passage of prostate spheres derived from Hi-Myc mice revealed that Hi-Myc prostate epithelial cells displayed significantly enhanced stem/progenitor self-renew capacity and growth compared to cells from WT mice (FIG. 11F). Strikingly, low dose of I3C treatment drastically suppressed Hi-Myc prostate sphere-forming ability; largely as a result of apoptosis induction (FIG. 11F).

Consistent with the data above, MYC expression not only induced WWP1/NEDD4-1 expression, but also AKT activation without affecting the PTEN protein level (FIG. 11G). Additionally, treatment with low dose of I3C abolished AKT activation driven by MYC expression, and led to a concomitant apoptosis in Hi-Myc prostate spheres, but not in WT prostate spheres (FIG. 11G). Taken together, in various cellular models, these data demonstrate that Hi-MYC expression induced “oncogenic addiction” to the WWP1/NEDD4-1/PTEN pathway, thus sensitizing cells to a low dose I3C treatment.

### Example 10—Therapeutic Targeting of K27-Linked PTEN Poly-Ubiquitination In Vivo

To examine the functional relevance of the WWP1/NEDD4-1/PTEN crosstalk in vivo, preclinical studies with I3C in Hi-Myc mice were performed. The expression of Hi-Myc in mouse prostate results in complete penetrance of prostatic intraepithelial neoplasia (PIN) and progresses into invasive adenocarcinoma within 5 to 12 months of age. To this end, cohorts of Hi-Myc mice at 5 months of age were treated with either the vehicle or I3C for one month.

It was found that vehicle-treated Hi-Myc mouse prostates displayed heterogeneous disease progression, among which dorsal lateral (DLP) prostate displayed extensive invasive carcinoma, whereas, ventral (VP) and anterior (AP) prostates developed PIN lesion to a similar extent (FIG. 12A). Notably, I3C-treated Hi-Myc mouse prostates had significantly reduced tumor size in both DLP and AP, albeit less prominently in VP (FIG. 12B).

Histological analyses confirmed that I3C-treated APs from Hi-Myc mice were completely disease-free, displaying normal-like glandular structure lined with a single layer of epithelial cells, while the DLPs displayed significantly lower penetrance of the invasive carcinoma (FIG. 12A). Consistent with the histopathological analyses, I3C treatment robustly inhibited AKT activation without affecting PTEN protein level in prostate lysates derived from both Hi-Myc AP and DLP, but not VP (FIG. 12C). Lastly, immunohistochemistry (IHC) analyses of PTEN protein in DLP tissue revealed that I3C administration induced intense PTEN plasma membrane accumulation in prostate epithelial cells compared to vehicle treatment (FIG. 12D). Thus targeting of WWP1/NEDD4-1 E3 by I3C triggers re-activation of the PTEN tumour suppressive function by promoting its plasma membrane recruitment to suppress MYC-driven prostate cancer in vivo.

Several conclusions may be drawn from the data presented in the preceding examples:

i) A new pathway in which MYC/WWP1/NEDD4-1-mediated PTEN K27-linked ubiquitination controls PTEN dimer formation and membrane recruitment that in turn suppresses its activity has been identified. This regulatory pathway does not trigger PTEN proteasomal degradation, which is consistent with the notion that K27-linked is an inefficient proteolytic signal (FIG. 12E). This study not only provides the first mechanistic insight into the control of PTEN dimerization and consequent PI3K signaling activation, but also attributes a distinct molecular function to the K27-linked ubiquitin chain in the control of protein dimerization. In line with these data, a previous study indicates that TRAF6 ubiquitin ligase targets AKT for K63-linked poly-ubiquitination to potentiate its membrane recruitment and activation. Non-proteolytic ubiquitination of the key components of the PI3K/AKT signaling pathway might therefore represent a general mechanism for regulating their membrane recruitment.

ii) These data also provide a coherent and unifying working model on how PTEN subcellular localization and function is regulated. It has been previously shown that NDFIP1 binds to PTEN and enhances its mono-ubiquitination by NEDD4-1 under ischemia conditions, triggering PTEN nuclear/cytosol shuttling. Here, it was found that NEDD4-1 could complex with WWP1 to induce PTEN K27-linked poly-ubiquitination, negatively regulating PTEN dimerization, moreover, the catalytic activities of both WWP1 and NEDD4-1 are required for the complete activation of this E3 ligases. Importantly, by examining the status of PTEN ubiquitination under various physiological stimuli, such as insulin, serum, and hypoxia, it was found that hypoxia could not only inhibit poly-ubiquitination, but also determine the mono-ubiquitin chain specificity conjugated on PTEN by NEDD4-1 (FIG. 13A, FIG. 13B, and FIG. 13C). These findings in turn suggested that the NEDD4-1 E3 ligase may interact with different partners to determine the substrate chain specificity: NDFIP1 towards mono-ubiquitination; WWP1 towards K27-linked poly-ubiquitination. In line with this notion, it was found that while in normoxic conditions NEDD4-1 could complex with WWP1 to promote PTEN K27-linked poly-ubiquitination, in response to hypoxia, NEDD4-1 switched its interaction partners to NDFIP1, increasing PTEN mono-ubiquitination (FIG. 13C, FIG. 13D). Intriguingly, depletion of NDFIP1 restored PTEN poly-ubiquitination under hypoxia condition, pointing to a crucial role of NDFIP1 together with NEDD4-1 in determining PTEN mono-ubiquitination (FIG. 13C). These findings thus provide another layer of mechanistic regulation of E3-ubiquitn ligases to increase their versatility.

iii) These data also resolve a long-standing argument in tumour biology regarding the function of NEDD4-1 towards PTEN. While NEDD4-1 mediated PTEN degradation still remains largely controversial, these data show that the WWP1/NEDD4-1 is a potent upstream negative regulator of PTEN that can oppose PTEN cytosolic and nuclear function through K27-linked poly-ubiquitination, rather than the canonical K48 chain that promotes protein degradation. In agreement with these findings, the presence of the WWP1/NEDD4-1 strongly correlates with PTEN off-membrane re-localization without affecting its protein level, which is consistent with the data showing that endogenous PTEN protein stability was not affected upon overexpression of WWP1 or NEDD4-1, at the steady state or when assessed by cyclohexamide pulse-chase analysis (FIG. 5 and FIG. 1D). Genetically, CRISPR-mediated WWP1 knockout or genetic ablation of NEDD4 robustly induced PTEN dimerization and in turn inhibited AKT activity without affecting PTEN protein level (FIG. 5F, FIG. 5G, and FIG. 5I). Together, these data strongly suggest that PTEN proteasomal degradation is controlled by other E3 ligases, such as WWP2; or alternatively, that NEDD4-1 may form additional E3 complexes with other E3 ligases to promote PTEN ubiquitination and degradation in other cell types or under different physiological conditions.

iv) WWP1 has been implicated in the regulation of various signaling processes involved in tumour proliferation and apoptosis. Here, these data have not only uncovered that WWP1 is a new target of MYC, but also identified that WWP1 is co-amplified with MYC in human CaP specimens. Mechanistically, these data have unraveled the oncogenic function of WWP1 towards PTEN. Genetic ablation of WWP1 by CRISPR/Cas9 gene editing approach not only induced PTEN dimerization and membrane recruitment, but also robustly inhibited AKT activation (FIG. 5F-H), corroborating the crucial roles of WWP1 in PTEN suppression. Thus, these data not only provide the first functional linkage of WWP1 to PTEN, but also identify a critical therapeutic target for PTEN reactivation and cancer therapy.

v) Given that overexpression of WWP1 or NEDD4-1 is very frequently observed in human cancer of various histological origins, the impact of this PTEN suppressive pathway may well be widely prevalent. Strikingly, deregulated MYC overexpression induces “oncogenic addiction” of cells to the WWP1/NEDD4-1/PTEN axis. When the WWP1/NEDD4-1 E3 pathway is inhibited in “addicted” cells through knockdown (shRNAs) or compound treatment (I3C), the survival and growth of cancer cells are suppressed. Since PTEN is frequently down-regulated or mono-allelically lost in human cancer, the pharmacologic blockage of this pathway by targeting of WWP1 and/or NEDD4-1 represents an exciting therapeutic strategy to treat MYC-driven tumours through PTEN reactivation. Intriguingly, this therapeutic approach may extend to “PTEN mutant cancers” as well, since WWP1/NEDD4-1 do trigger AKT super-activation even in the presence of mutant PTEN (FIG. 14A and FIG. 14B). Conceptually, these findings pave the way towards a long-sought tumour suppressor mediated therapy for cancer prevention and treatment.

The results described herein were obtained with the following materials and methods.

Murine Models.

The Beth Israel Deaconess Medical Center IACUC Committee on Animal Research approved all animal experiments. The transgenic mice used in these studies (Hi-Myc-mice), in which the prostate specific expression of human c-Myc is driven by the rat probasin promoter with two androgen response elements, were obtained from the Mouse Repository of National Cancer Institute. 9 mice per genotype were randomly chosen and used to examine the tumour at the indicated age. Wwp1−/−mice and its paired Wwp1+/+ mice (on a C57/BL6 background) were obtained from Dr. L. Matesic (University of South Carolina, Columbia, SC, USA) (Shu et al., 2013). The pathologist determined the histological grade blindly.

Plasmids, Antibodies and Reagents.

Human PTEN cDNA was cloned into the pLVX-Puro vector to generate a PTEN lentivirus expression plasmid (Clontech). LentiCRISPR v2 plasmid was a gift from Feng Zhang (Addgene #52961). pCDH-puro-cMyc (46970), HA-human NEDD4-1 WT (24124), HA-NEDD4-1 C867A (24125) and were purchased from Addgene. Flag-WWP1 ΔWW domain (deletion 341-547) were generated by Q5 Site-Directed Mutagenesis Kit (E0544S), whereas all mutant constructs of PTEN were generated using a QuickChange Lightning Site-Direct Mutagenesis (Agilent Technologies). All mutations were confirmed by sequencing. His-Ubiquitin, His-Ubiquitin KR mutants, His-Ubiquitin K-only mutants, Myc-PTEN and its deletion constructs have been previously described. pcDNA3-HA-MYC, pcDNA3-Myc-PTEN, pcDNA3-Myc-PTEN 4A, pcDNA3-Myc-PTEN 4E, pcDNA3-HA-PTEN, pcDNA3-HA-PTEN N-terminal (1-187), pcDNA3-HA-PTEN-C terminal (188-403), MYC-WWP1 WT, MYC-WWP1 C890A, and Flag-NEDD4 family ligases constructs, such as Flag-NEDD4-1, Flag-WWP1, Flag-WWP2, Flag-Smurf2, and Flag-Itch were gifts from Dr. Wei's lab. The two individual siRNA duplexes targeted to NEDD4-1, WWP1, Trim27, ITCH, RNF168, NDFIP1 and control non-target siRNA were purchased from Sigma Aldrich, while siRNA SMARTpool targeted to MYC was purchased from Dharmacon. Lentivirus based constructs expression shRNAs targeting human WWP1 (TRCN0000003398), NEDD4-1 (TRCN0000007553) and PTEN (TRC0000002746; TRC0000002747) were obtained from GE Dharmacon. Lipofectamine 2000, RPMI, DMEM, Opti-MEM reduced serum media and fetal bovine serum (FBS) were purchased from Invitrogen. Anti-Flag-M2 affinity gel, insulin, and puromycin were purchased from Sigma Aldrich. Insulin was used at 100 or 200 ng/ml. Polybrene was purchased from Santa Cruz Biotechnology, Inc. Indole-3-carbinol (I3C) was purchased from Sigma Aldrich. For western blotting: Anti-Myc-Tag (2276), anti-PTEN (9559), Anti-MYC for western blot and ChIP assay (13987), anti-NEDD4 (2740 and 3607), anti-EGFR (4267), anti-Ubiquitin (3936), anti-Cleaved Capase3 (9661), anti-Phospho-AKT (pSer473, 9271; pThr308, 9275), anti-AKT (pan AKT, 4685) antibodies were all purchased from Cell Signaling Technology. Mouse Anti-PTEN antibody (6H2.1) was purchased from Cascade Bioscience; Anti-GFP (A-11120) was purchased from Invitrogen; Anti-WWP1 (human) (H00011059-M01) was purchased from Novus Biologicals; Anti WWP1 (mouse) (13587-1-AP) was purchased from Proteintech; Anti-Actin (A3853 and Anti-Flag-M2) were purchased from Sigma Aldrich; Anti-HA-Tag (16B12) was purchased from Covance. For immunohistochemistry in the tissue microarray (TMA) analysis: anti-PTEN (9559) was purchased from Cell Signaling Technology; anti-NEDD4 (07-049) was purchased from Millipore.

Cell Culture, Transfection and Establishment of Stable Cell Lines.

All cell lines were obtained from ATCC and checked for mycoplasma by MycoAlert Mycoplasma Detection Kit (Lonza). Nedd4^{-/-} MEFs and paired Nedd4^{+/+} MEFs were kindly provided by Dr. B. Yang (University of Iowa). Wwp1^{-/-} MEFs were generated by the CRISPR/Cas9 gene editing approach. 293, 293T and primary MEF cells were maintained in DMEM supplemented with 10% fetal bovine serum, 2 mM glutamine, 100 U/ml penicillin and streptomycin (Invitrogen). PC3, LNCaP, C4-2, 22rv1 and VCaP cells were cultured in RPMI medium containing 10% fetal bovine serum, 2 mM glutamine, 100 U/ml penicillin and streptomycin (Invitrogen). RWPE-1 and PWRE-1 cells were cultured in a K-SFM medium supplemented with recombinant human Epidermal Growth Factor (rhEGF) and Bovine Pituitary Extract (BPE). Transfections were performed using Lipofectamine 2000 reagent (Invitrogen) according to the manufacturer's instruction. In brief, 5×10⁵ cells were transfected with 5 μg of DNA plasmids or 20 nM of siRNA in a 6-well dish. Cells were recovered into completed media after a 12 hour transfection and then harvested at the indicated times. Stable cell lines were generated by lentivirus transduction.

Lentivirus Production and Infection.

To generate recombinant lentivirus, 293T cells were co-transfected with VSVG, PMDL, REV, and indicated lentivirus based constructs. The virus-containing supernatant was harvested. For infection, the viral stock was supplemented with 10 μg/ml of polybrene and the infected cells were selected by 2 μg/ml of puromycin.

Cell Proliferation Assay.

At 8 hours post-transfection, cells were trypsinized, resuspended, and seeded in three separate 12-well plates at a final density of 20,000/well. Starting the following day (d0), one plate per day was washed once with PBS, fixed in 10% formalin solution for 10 minutes at room temperature, and kept in PBS at 4° C. On the last day, all of the wells were stained with crystal violet. After lysis with 10% acetic acid, optical density was read at 595 nm.

In Vivo Ubiquitination Assay.

To analyse in vivo ubiquitination of PTEN, cells were transfected with various constructs, together with His-Ubiquitin and Myc-PTEN. Cells were lysed by buffer A (6 M guanidine-HCl, Na₂HPO4/NaH₂PO4 [pH 8.0], and 10 mM imidazole), and lysates were incubated with Ni-NTA agarose for 1.5 hours at 4° C. The beads were washed once with buffer A, twice with buffer ANTI (1 vol buffer A: 3 vol buffer TI [25 mM Tris-HCl, pH 6.8, and 20 mM imidazole]), and three times with buffer TI, and then analysed by western blot. In all experiments, an equal amount of His-Ubiquitin expression was verified by western blot analysis.

In Vitro Ubiquitination Assay.

For in vitro ubiquitination, 400 ng of purified Flag-PTEN from 293 cells was incubated with 40 ng E1 (E-305 Boston Biochem), 500 ng E2 (E2-616 Boston Biochem), 10 μg His-Ub variants (Boston Biochem), 8 mM ATP, 1×ligase reaction buffer (Fisher Scientific (Thermo Fisher Scientific) # B71), and 1×Energy regeneration system (Boston Biochem #B-10), 400 ng NEDD4 (Sigma Aldrich # SRP0226) and 400 ng WWP1 (Sigma Aldrich # SRP0229) at 37° C. for 1 hour in 25 ul reaction mixture.

Cell-Cycle Profile Analysis by Flow Cytometry.

DU145 stably expressing indicated constructs were collected. The cells were fixed by 75% ethanol at −20° C. overnight and washed 3 times using cold PBS. The samples were treated with 1 ug/ml RNase for 30 minutes at 37° C. and stained with 5 ug/ml propidium iodide (Roche) on ice for 1 hour. Stained cells were sorted with BD FACSCanto™ II Flow Cytometer. The results were analysed by FlowJo softwares.

Mass Spectrometry.

DU145 cells transfected with HA-PTEN were immunoprecipitated with anti-PTEN antibody and the PTEN-associated proteins were resolved by SDS-PAGE on 4%-12% gradient gel (Invitrogen) for coomassie blue staining. Specific bands were cut out from the gel and subjected to mass-spectrometric peptide sequencing.

Western Blotting and Immunoprecipitation. For western blotting, cells were lysed in RIPA buffer (Boston BioProducts) supplemented with protease (Roche) and phosphatase (Roche) inhibitor. Proteins were separated on NuPAGE 4-12% Bis-Tris gradient gels (Invitrogen), transferred to polyvinylidine difluoride membranes (Immobilon P, Millipore) and the blots were probed with the indicated antibodies. For immunoprecipitation, PC3, 293T and MEF cells were transfected with the indicated expression vectors by using LIPOFECTAMIN 2000 (Life Technologies). 24 hours after transfection, cells were lysed in RIPA buffer with protease (Roche) and phosphatase (Roche) inhibitor. 500 μg of total lysates were pre-cleared for 30 minutes at 4° C., and then immunoprecipitated with anti-Myc (Cell Signaling Technology 9B11, 1:500), or anti-PTEN (Cell Signaling Technology 9559, 1:500) antibody overnight at 4° C. The Protein-A or Protein-G sepharose beads (GE Healthcare) were then added and incubated for another 2 hours. The immunoprecipitates were washed with RIPA buffer three times. In denaturing conditions, standard Laemmli-Buffer with 5% final concentration of β-mercaptoethanol was added to the samples, which were then boiled and separated on NuPAGE 4-12% Bis-Tris gradient gels (Invitrogen). In non-reducing conditions, cells were lysed in lysis buffer containing 20 mM Tris-HCl pH7.5, 150 mM NaCl, 1% NP40, 1 mM EDTA, 1 mM protease (Roche) and phosphatase inhibitor (Roche) for further immunoprecipitation. For the native elution, pre-chilled 0.1 M glycine pH 2.5 was used to elute immunocomplexes for 10 minutes at 4° C., further neutralized with 1 M Tris-HCl pH 8.0. Laemmli-Buffer without reducing agents was added and samples were immediately run on NuPAGE 4-12% Bis-Tris gradient gels (Invitrogen).

NanoLC-MS/MS Analysis of Ubiquitinated PTEN.

Ubiquitinated PTEN was isolated by anti-Flag M2 beads from cells transfected with Flag-PTEN, His-ubiquitin, together with or without WWP1/NEDD4-1. The bound proteins were eluted with denaturing buffer containing 8 M urea, 20 mM HEPES, 1 μg/ml aprotinin, 1 μg/ml leupeptin, 1 mM N-ethylmaleimide (Sigma), and 1 mM PMSF. Eluted proteins were digested with trypsin for overnight, treated with trifluoroacetic acid (TFA), and clarified by centrifugation. The supernatant was desalted on a Sep-Pak C18 column (Waters), and lyophilized peptides were dissolved in IP buffer containing 50 mM MOPS, pH 7.2, 10 mM sodium phosphate and 50 mM NaCl. Ubiquitinated peptides were enriched by incubation overnight with protein A agarose conjugated with an anti-K-ε-GG antibody (Cell Signaling Technology, Inc), which specifically recognizes the di-glycyl remnant produced on ubiquitinated lysine residues after trypsin digestion. Beads were washed with IP buffer followed by water, and bound peptides were eluted with 0.15% TFA, desalted by Stage tip chromatography, and lyophilized for MS analysis.

NanoLC-nanoESI-MS/MS analysis was performed on a nanoAcquity system (Waters) connected to the Orbitrap Elite hybrid mass spectrometer (Thermo Electron) equipped with a PicoView nanospray interface (New Objective). Peptide mixtures were loaded onto a 75 μm ID, 25 cm length C18 BEH column (Waters) packed with 1.7 μm particles with a pore of 130 Å and were separated at 35° C. using a segmented gradient from 5% to 35% acetonitrile in 0.1% formic acid at a flow rate of 300 nl/min for 90 min. The mass spectrometer was operated in the data-dependent mode. Briefly, surveying full scan, MS spectra were acquired in the orbitrap (m/z 350-1600) with the resolution set to 120K at m/z 400 and automatic gain control (AGC) target at 106. The 15 most intense ions were sequentially isolated for HCD MS/MS fragmentation and detection in the orbitrap with previously selected ions dynamically excluded for 60 seconds. For MS/MS, we used a resolution of 15000, an isolation window of 2 m/z and a target value of 50000 ions, with maximum accumulation times of 200 ms. Fragmentation was performed with normalized collision energy of 30% and an activation time of 0.1 ms. Ions with singly and unrecognized charge state were also excluded. All data generated were searched against the customized Swiss-Prot Homo sapiens database and His-tagged ubiquitin protein sequences (20,169 entries total) database using the Mascot search engine (v.2.5.1; Matrix Science, Boston, Mass., USA) through Proteome Discoverer (v 2.1.0.81; Thermo Scientific). Search criteria used were trypsin digestion, variable modifications set as carbamidomethyl (C), oxidation (M), GlyGly (K) and LeuArgGlyGly (K) allowing up to 2 missed cleavages, mass accuracy of 10 ppm for the parent ion and 0.02 Da for the fragment ions. Two target values for a decoy database search were applied: strict FDR of 0.01 and a relaxed FDR of 0.05. Ubiquitination sites and peptide sequence assignments contained in MASCOT search results were validated by manual confirmation from raw MS/MS data. For label-free quantification, precursor ions areas were extracted using Precursor Ions Area Detector node in Proteome Discoverer 2.1.0.81 with a 2 ppm mass precision (the experimental m/z and retention times were recorded for precursor area quantification).

Chip Assay.

DU145 cells were fixed by addition of 37% formaldehyde to a final concentration of 1% formaldehyde and incubated at room temperature for 10 min. Crosslinking was stopped by the addition of glycine to a final concentration of 0.125 M. Cells were then scraped, and samples were prepared using SimpleChIP Enzymatic Chromatin IP Kit (Cell Signaling, #9003) according to the manufacturer's protocol. The chromatin fractions were incubated in each case with 10 mg of antibodies to one of the following: MYC (Cell Signaling, #13987), human RPL30 or normal rabbit IgG (both provided by Cell Signaling kit, Cell Signaling) at 4° C. overnight with Magnetic Protein G Beads. After extensive washing and final elution, the product was treated at 65° C. overnight to reverse the crosslinking. Input DNA and immunoprecipitated DNA were purified using a kit column and analysed by qPCR using SYBR Green Supermix (Bio-Rad) with the following sets of primers (both proximal and distal promoter regions): human WWP1 promoter (forward, 5′-GTCCGGAGTTGGAGGCTTT-3′ (SEQ ID NO: 4); reverse, 5′-GACCCCACACCTCCCTTC-3′ (SEQ ID NO: 5)), human NEDD4-1 promoter (forward, 5′-CCGTCAACCACCCACCTC-3′ (SEQ ID NO: 6); reverse, 5′-CTCCCTCAGCGACAGCAG-3′ (SEQ ID NO: 7)), human JunB (forward, 5′-AAGCCCACAGAGAGAGGTGGAAG-3′ (SEQ ID NO: 8); reverse, 5′-CCAGAAGGTGGTGCCTTTTTATTG-3′ (SEQ ID NO: 9)). All results were normalized to the respective input values.

CRISPR/Cas9 Gene Editing Approach.

Construction of lenti-CRISPR/Cas9 vectors targeting WWP1 in MEFs was performed following the protocol associated with the backbone vector (Addgene, #52961). The software (http://crispr.mit.edu/) predicted the following sequences with priority given to sequences that matched the early coding exons of targeted genes. The non-bold part of the sequence is gene-specific. WWP1 sgRNA_1 (fwd: 5′-CACCGATCAGCTGCTCGTCCCATTT-3′ (SEQ ID NO: 10); rv: AAACAAATGGGACGAGCAGCTGATC) (SEQ ID NO: 11), WWP1 sgRNA_2 (fwd: CACCGTAATACTCGAACTACTACAT (SEQ ID NO: 12); rv: AAACATGTAGTAGTTCGAGTATTAC (SEQ ID NO: 13)).

Cellular Fractionation.

Membrane versus cytosolic fractionation of 293T, MEF, or PC3 cells transfected with the indicated constructs was performed using the ProteoExtract Native Membrane Protein Extraction Kit (Calbiochem), and according to the manufacture's procedures.

Gel Filtration Chromatography.

293 cells were transfected with the indicated expression vectors. After 24 hours, cells were washed with PBS, lysed and filtered through a 0.45 μm syringe filter. 500 μl of lysate (4 mg/ml) was loaded into a Superdex200 10/300 GL column (GE Lifesciences Cat. No. 17-5175-01). Tandem columns gel filtrations were performed by further attaching a series Superdex 75 10/300 GL column (GE Lifesciences Cat. No. 17-5174-01). Samples were separated in a Superdex75 column first, followed by a Superdex 200 column. Chromatography was performed by using anAKTA FPLC (GE Lifesciences Cat. No. 18-1900-26), and protein complexes were resolved and eluted with lysis buffer at 0.5 ml/minute, 500 μl per fraction. 40 μl aliquots of each fraction were separated by SDS-PAGE and western blot analysis was performed with the indicated antibodies. Before sample separation, molecular-weight resolution of the columns was estimated by running the Gel Filtration Calibration Kit (GE Lifesciences, #28-4038-42) to determine retention times on Coomassie-stained SDS-PAGE protein gels.

Immunofluorescence Analysis.

PC3 and DU145 cells stably expressing the indicated constructs were plated on coverslip. The following day, cells were washed with ice-cold PBS, fixed in 4% paraformaldehyde, permeabilized with 0.02% Triton X-100, and then blocked with PBS supplemented with 20% goat serum. Cells were incubated with anti-PTEN antibody (6H2.1, 1:100) from Cascade Bioscience, diluted in PBS containing 10% goat serum overnight and then with Alexa488-conjugated secondary antibody together with 1 μg/ml of DAPI for 1 hour. Images were acquired with a LSM510META Confocal Laser System at the BIDMC microscopy core facility.

qPCR Analysis.

Quantitative real-time PCR was performed using the Power SYBR Green PCR Master Kit (Applied Biosystems). Amplification was performed on an ABI 7500 Fast Real-Time PCR system and actin was used as the internal control. The PCR primers used were WWP1 forward: 5′-TGCTTCACCAAGGTCTGATACT-3′ (SEQ ID NO: 14), WWP1 reverse: 5′ GCTGTTCCGAACCAGTTCTTTT-3′ (SEQ ID NO: 15); Trim27 forward: 5′-AGCCCATGATGCTCGACTG-3′ (SEQ ID NO: 16), Trim27 reverse: 5′-GGGCACGACACGTTAGTCT-3′ (SEQ ID NO: 17); Itch forward: 5′-TGATGATGGCTCCAGATCCAA-3′ (SEQ ID NO: 18), Itch reverse: 5′-GACTCTCCTATTTTCACCAGCTC-3′ (SEQ ID NO: 19); RNF168 forward: 5′-GGATCTGCATGGAAATCCTCG-3′ (SEQ ID NO: 20), RNF168 reverse: 5′-ACTGGAAGCACGGTTTACACA-3′ (SEQ ID NO: 21); human actin forward: 5′-CTCTTCCAGCCTTCCTTCCT-3′ (SEQ ID NO: 22), human actin reverse: 5′-AGCACTGTGTTGGCGTACAG-3′ (SEQ ID NO: 23).

Soft-Agar Colony-Formation Assay and Xenotransplantation.

For assaying colony formation in soft agar, 1.5×10⁵ PC3 derivatives were re-suspended in 0.3% top agar. Colonies formed after 3 weeks were stained by crystal violet and counted. For assaying tumour growth in the xenograft model, 7-week-old NCr nude mice housed in specific pathogen-free environments were injected s.c. with 2.5×10⁶ PC3 derivatives (n=5 for each group) mixed with RPMI medium and Matrigel (vol/vol, 1:1).

Immunohistochemistry (IHC) Assay.

Individual tumours derived from NCr nude mice were dissected and fixed in 4% paraformaldehyde for IHC analysis. For staining, the tissues were embedded in paraffin according to standard procedures. 5 μm sections were cut and processed for H&E staining or were stained for PTEN (Cascade BioScience 6H2.1, 1:250). The stained slides were visualized by a bright-field microscope.

Tissue Microarray (TMA) Analysis.

The TMAs used in this study were constructed at the Memorial Sloan-Kettering Cancer Center (MSKCC). The study cohort was comprised of radical prostatectomy specimens from 126 patients with primary CaP. Tumour samples were collected at the time of surgical resection with written informed consent. The patients were treated and their progress was followed at MSKCC. PTEN (Cell Signaling Technology), and NEDD4 (Millipore) staining were performed as previously described²¹. Cases containing more than 50% of the core composed of tumour cells were analysed.

I. P. Administration.

The mice were treated I.P. with I3C dissolved in 5% DMSO (20 mg/kg), three times a week for 14 days starting on day zero. I.P. administration allows I3C to achieve maximal systemic exposure.

Gene Expression Profiling.

TCGA (Provisional) and MSKCC human prostate adenocarcinoma gene expression data sets were downloaded from cBioPortal (http://www.cbioportal.org/public-portal). For the analyses, GraphPad Prism 6 software was used and the analysis of correlation was done by Pearson correlation coefficients.

Statistical Analysis.

For analysis of average data, datasets were compared using unpaired two-tailed Student's t-tests. For the correlation of TMA staining with clinical parameters, datasets were compared using Pearson Chi-Square correlation. P values of <0.05 were considered to be statistically significant. All statistical tests were executed using GraphPad Prism software.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating cancer in a selected subject, the method comprising administering to the subject an effective amount of an agent that inhibits the formation of a NEDD4-1/WWP1 heterodimer, wherein the subject is selected by a method comprising detecting increased expression in MYC, NEDD4-1 or WWP1 relative to a reference.

2. (canceled)

3. A method of inhibiting neural precursor cell expressed developmentally down-regulated protein 4 (NEDD4-1) and WW domain-containing protein-1 (WWP1) in a neoplastic cell, the method comprising contacting the cell with an agent that inhibits the formation of a NEDD4-1/WWP1 heterodimer.

4. A method of inhibiting the survival or proliferation of a neoplastic cell having increased MYC expression, the method comprising contacting the cell with an agent that inhibits the formation of a NEDD4-1/WWP1 heterodimer, wherein the cell is characterized as having increased MYC expression, thereby inhibiting the survival or proliferation of the neoplastic cell.

5. The method of claim 3, wherein the neoplastic cell is a mammalian cell.

6. The method of claim 5, wherein the mammalian cell is a murine, rat, or human cell.

7. The method of claim 5, wherein the cell is in vitro or in vivo.

8. The method of claim 4, wherein the neoplastic cell or cancer comprises a mutation in PTEN.

9. The method of claim 4, wherein the neoplastic cell or cancer overexpresses cMYC.

10. The method of claim 4, wherein the method reduces neoplastic cell survival or proliferation.

11. The method of claim 4, wherein the neoplastic cell is derived from prostate cancer, breast cancer, or colorectal cancer.

12. The method of claim 1, wherein the subject has prostate cancer, breast cancer, or colorectal cancer.

13-26. (canceled)
